SEQUENCE LISTING (17 sequences)

SEQ ID NO: 1 - DNA, 6471 bp, Artificial Sequence
  Synthetic
atggtgaacgagaacacaaggatgtatatcccagaagagaatcatcagggctccaactacggttctccaagacctgcacacgccaacatgaacgctaacgcagctgctggactggcaccagaacacatccctactccaggagctgcactgtcctggcaggctgctatcgatgctgctaggcaggctaagctgatgggctcagctggcaatgcaaccatcagtactgtttccagtacccagcgcaaacgccagcagtatgggaagccaaagaagcagggctccaccactgcaactagaccacctagggcactcctctgtctcaccctcaagaatcctattcgcagggcttgcatttctatcgtggagtggaaacccttcgaaatcatcattctgctgaccatctttgctaactgtgtcgctctcgctatctatatcccatttcctgaggacgattctaatgcaaccaactctaatctggagagagtggagtatctcttcctcatcatcttcacagtggaagccttcctgaaagtgattgcctatggcctcctctttcatccaaacgcttacctgcgcaacggatggaacctgctcgacttcatcattgtggttgtcggactgtttagtgccattctggagcaggccacaaaggctgacggagccaatgctctgggtgggaagggtgctggctttgatgtcaaggctctgagagctttcagagtcctgaggcctctgaggctcgtgtctggcgtcccaagcctgcaggtcgtgctgaactctatcatcaaagccatggtccctctgctccacattgctctcctggtcctgttcgtgatcattatctacgcaatcatcggtctggaactgtttatgggaaagatgcataagacatgctacaaccaggaagggattgccgatgtgcctgcagaggacgatcctagcccatgcgcactggaaactgggcacggacggcagtgccagaacggaactgtgtgcaaacctggctgggacggtccaaagcatggcattactaactttgataacttcgccttcgccatgctcacagtcttccagtgcattactatggaaggatggacagacgtcctgtactgggtgaacgatgctgttggacgggattggccttggatctacttcgtgaccctgatcatcattgggtcattctttgtcctgaacctcgtgctcggagtcctcagtggagagttctccaaggagagggagaaggccaaagccagaggagacttccagaagctcagggagaaacagcagctcgaagaggatctcaagggatacctcgattggatcacacaagcagaggacatagatccagagaacgaggatgagggaatggacgaagagaaaccacggaatatgtctatgcccacatcagaaaccgaaagtgtgaataccgagaacgtggcaggtggcgacatcgaaggtgagaactgtggagcacggctggcacacagaatatctaagagcaaattctcaaggtactggagaaggtggaatagattctgcaggaggaagtgtagagctgcagttaagtctaacgtgttctactggctcgtgatctttctcgtgttcctgaatactctcaccatagccagcgagcactacaaccagcccaattggctgaccgaggtccaagacaccgctaacaaggctctcctggcactgttcacagctgaaatgctgctgaagatgtatagcctgggtctgcaggcatacttcgtctccctcttcaatcggtttgactgctttgtcgtgtgtggtggaatcctcgaaaccatcctggtggaaaccaagatcatgtcaccactcgggatctctgttctccgctgtgttcgcctgctgcggatcttcaagatcactcggtattggaactccctctctaatctggtggccagtctcctcaatagtgtgaggtccattgcttctctgctcctcctgctgttcctgttcattatcatctttagtctgctgggaatgcagctcttcggtgggaaattcaacttcgatgagatgcagacaagacactacccttatgatgtccctgattatgctgtcacctttgatgaaatgcagaccaggagaagcacatttgataactttccacagagtctgctgaccgtgttccagattctgacaggagaagactggaactcagtgatgtacgacggcatcatggcatacggaggtccctcatttcctggaatgctggtttgtatctacttcatcatcctgtttatctgtgggaattacatcctgctcaatgtgttcctggccattgccgtggacaacctggctgacgctgaaagcctgacctccgctcagaaggaggaagaggaggagaaagaaaggaagaagctggcacggacagccagtccagagaagaagcaagaactcgtggagaaaccagcagtgggagaaagcaaggaggagaagatcgaactcaaatccatcacagccgatggcgagagcccacctgccactaagattaacatggacgatctccagccaaacgagaatgaggataagtctccatatccaaatcccgagacaactggcgaggaggacgaagaggagcctgagatgcctgttggacccaggcctagacctctctcagagctgcacctgaaagagaaagccgtgcccatgccagaggcttctgctttcttcatcttctccagcaacaatcgctttcggctgcaatgccaccgcatcgtcaatgacacaatcttcaccaacctcattctgttctttatcctcctctcttccatcagtctcgctgctgaagaccctgtccagcatacatccttccggaatcacatcctgggcaatgccgactacgtgttcactagcatctttacactggagattatcctgaagatgacagcctacggtgccttcctgcacaaagggtccttctgcaggaactacttcaatatcctggatctgctggtcgtctctgtgtctctcattagctttggtatacaaagttcagcaatcaacgtcgtcaagattctgagggtgctgagagttctgaggccactgagggcaatcaatagagccaaaggactcaagcacgtggtccagtgcgtgttcgttgcaatccggaccattggaaacatcgtgatcgtgactactctgctgcagtttatgttcgcatgtattggagtgcagctcttcaagggaaagctgtatacctgctctgacagttccaagcagaccgaggcagaatgcaagggtaactacatcacttacaaggatggcgaagtggatcatccaatcatacagccacgcagttgggagaacagcaagtttgacttcgacaacgtgctggctgccatgatggctctcttcaccgtgtccacattcgaaggatggcctgaactcctctatagaagcatcgattctcatactgaggacaagggacccatctataactatcgggtcgagatttcaatcttcttcatcatatacatcatcatcatcgccttcttcatgatgaacatcttcgtgggcttcgtcatcgttacattccaggagcaaggcgagcaggagtacaagaattgcgaactcgacaagaaccagaggcagtgtgtggaatatgccctcaaagcaaggcctctcagacggtacatcccaaagaaccaacatcaatacaaagtctggtacgtggtcaactccacctactttgagtacctgatgtttgtcctgatactgctcaatacaatatgtctcgccatgcaacactatgggcaaagttgtctgttcaagatagcaatgaacatcctgaatatgctgtttactggcctgttcaccgtggaaatgatcctgaagctgatcgctttcaagcctaagcattacttctgcgacgcctggaatactttcgacgctctcatcgtcgttggatcaatcgtggacatagccatcacagaagtgaacccagccgagcacacacagtgcagcccatccatgaacgctgaagagaatagcaggataagcatcacattctttagactctttcgcgtgatgagactggtcaagctgctgtccagaggagagggcataaggaccctgctctggacattcataaagagcttccaagcactgccctatgttgctctgctgatcgtgatgctgttcttcatctacgcagtcatcggcatgcaagtgttcggcaagatcgcactgaatgacaccaccgagatcaatagaaacaacaacttccagacctttcctcaggctgtgctgctgctgtttcggtgtgctactggagaggcttggcaggacatcatgctcgcatgcatgccagggaagaaatgcgcaccagagagtgaacctagcaactctaccgaaggcgaaacaccctgcggttcaagtttcgctgtcttctacttcattagcttctacatgctctgcgcatttctgatcataaacctctttgttgctgtcatcatggacaatttcgactacctgactagagattggtccatcctgggtccacatcacctggacgagttcaaacgcatctgggctgagtacgacccagaggccaaaggacgcatcaagcatctggacgtggtgacactgctcagacggattcaaccaccactgggctttggaaagctctgccctcatcgggtcgcttgcaaacgcctggtgagcatgaatatgcctctcaatagtgatgggaccgttatgtttaacgcaacactgttcgctctggtgcgcacagccctcagaatcaagactgaaggaaacctggagcaagccaatgaagaactcagagccattatcaagaagatttggaagaggactagcatgaagctgctggatcaggttgttcctccagctggcgacgatgaagtcaccgtgggcaagttctatgcaacatttctcattcaggaatactttcggaaattcaagaagaggaaggagcaaggactcgtcgggaaacccagccagcgcaatgccctcagtctgcaagctgggctcagaacactgcatgacattggtcctgagatacgcagagccatctctggcgatctgacagctgaagaggagctggacaaagcaatgaaggaagccgtgtccgctgcctccgaggacgacatattccgcagagctggtggcctcttcgggaaccacgttagctattaccagtccgatggaagaagtgctttcccacagaccttcaccactcagaggccactgcatatcaataaggctggcagcagccaaggcgatacagagagtccatcccatgagaagctggtggatagtaccttcactcctagtagctacagtagcacaggcagcaacgccaatatcaacaatgccaacaacaccgctctcgggaggctcccaagaccagcaggataccctagcaccgtctccaccgtggagggacatggaccacctctgagcccagccattagggtgcaagaagtggcctggaagctgtcttctaaccgctgccatagtcgcgagtcccaggcagccatggctggccaggaagaaacatcacaggacgaaacttatgaagtgaagatgaaccacgataccgaggcttgttccgagccctcactcctgagcaccgagatgctgtcataccaggatgatgagaataggcagctgaccctcccagaggaagacaaacgggacatcagacagagcccaaagagaggtttcctgagaagcgcttcactgggcaggagagccagctttcatctcgaatgtctgaaacgccagaaagacagaggtggcgacattagccagaagacagtgctgcctctgcacctggtgcaccatcaggcactggcagtcgctgggctgagcccactgctgcagaggtcacatagtcctgcctctttccctagacctttcgccactccaccagccactcctggaagcagaggctggccaccacagccagttcccactctcagactcgaaggtgtggaatcatccgagaagctcaatagctcattcccaagcatccattgtggctcttgggcagagacaactcctggaggtggtggaagctctgctgctaggagagttaggccagtttcactgatggtgccctcccaggctggtgcaccaggacggcagtttcacggatctgcatcctctctggtggaggcagtcctcattagcgaagggctgggacagtttgcccaggacccaaagttcattgaagtgacaacccaagagctggcagacgcctgtgacatgacaatagaagaaatggaatcagctgctgacaacattctgagtggaggagcaccacaaagtccaaatggagcactgctgcccttcgtcaactgtagggatgctggtcaggatagagcaggtggcgaggaggatgcaggatgtgttagggctagaggtagaccatctgaagaggaactgcaggactccagagtgtacgtgagcagcctg

SEQ ID NO: 2 - DNA, 6540 bp, Artificial Sequence
  Synthetic
atgatgatgatgatgatgatgaagaagatgcagcaccaaaggcagcaacaagcagatcacgcaaatgaggccaattacgccagagggaccaggctccctctctcaggagaaggtcctactagccagccaaactctagcaagcaaacagtgctgagttggcaggctgcaattgacgcagctagacaagccaaagcagcccagacaatgagtacatctgcaccacctcccgtcgggagtctgagccaaagaaagaggcagcagtatgccaagagcaagaagcagggtaatagcagtaacagcaggccagctagggcactcttctgtctgagcctgaataaccctattaggagggcttgtatcagtatcgtggagtggaagccattcgacatcttcatcctcctggctatcttcgccaactgtgtggcactcgcaatctacatacctttccctgaggacgactccaattccaccaaccacaacctggagaaagtcgaatacgccttcctcatcatattcactgtcgagacattcctgaagatcatcgcttacggactgctgctccatcccaacgcatacgtccggaacgggtggaacctgctcgacttcgtcattgtgatcgtgggactgttctccgtcatcctcgaacagctcactaaagagacagagggtggcaaccattcttctggaaagagcggaggattcgatgtcaaagctctgagagctttcagagtgctcagacctctgagactggtgtcaggagtgccatccctgcaggtggtgctgaactctatcatcaaagcaatggttccactcctgcacatagccctgctggttctgtttgtcatcatcatctacgccatcatcggtctggagctgttcataggcaagatgcacaagacctgcttctttgccgattcagatattgtggcagaagaggacccagctccatgtgcctttagtggtaatggacgccagtgcactgccaacggcactgaatgtcggtctggatgggtgggacctaatggaggcatcacaaactttgacaactttgcctttgccatgctcacagtgttccagtgcattaccatggagggctggacagacgtgctgtactggatgaatgacgcaatgggcttcgaactgccttgggtgtactttgtgtctctggttatctttggaagtttctttgtgctgaatctggttctcggagtgctgtctggagagttctctaaggaaagagagaaggccaaggctagaggcgacttccagaagctcagggagaagcagcagctggaggaggatctcaaagggtatctggattggataacccaagccgaggatattgaccctgagaacgaggaggagggaggcgaggaaggcaagagaaacacatcaatgccaaccagtgagactgagtctgtcaacaccgagaatgtctctggagagggtgagaatagaggatgttgtggctccctgtgccaagccatttctaagtctaagctgagcagaaggtggaggaggtggaatcggtttaaccgcagacggtgtagagctgctgttaagtcagttactttctattggctggtgatcgtgctggtctttctgaacaccctgacaatctcctctgaacactataaccagcctgactggctgacccagatacaggacattgccaacaaggtgctgctcgctctctttacttgcgaaatgctggtgaagatgtactcactcggactgcaggcctacttcgttagcctcttcaacagatttgattgctttgtcgtgtgtggaggaatcacagaaaccatcctcgtggaactggagatcatgagtcctctgggtataagcgtgttcaggtgcgtgagactgctgagaatcttcaaggtgactcggcactggacatctctctcaaacctcgtggcttccctcctgaacagtatgaaatccattgccagcctgctcctgctcctgttcctgttcatcatcatcttctccctgctcgggatgcagctgttcggtggaaagtttaactttgatgaaacccagacaagacactacccttatgatgtccctgattatgctgtcacctttgatgaaatgcagactaagaggagcacatttgacaatttcccacaagccctgctcactgtgtttcagatcctgacaggtgaagattggaacgctgtgatgtacgatgggatcatggcatacggaggtcctagtagctcagggatgattgtgtgcatctacttcatcatcctgtttatctgcggaaactacatcctcctgaatgtcttcctcgcaattgcagtggataatctggcagatgccgaatctctgaataccgcacagaaagaagaagctgaagagaaggagaggaagaagatcgctcggaaagaatctctggagaataagaagaacaacaagccagaggtgaatcagattgctaactcagacaacaaggtcaccatcgacgattatcgcgaggaggatgaggacaaggacccatatccaccctgcgacgtgcctgtcggagaagaagaagaggaagaggaggaggacgagccagaggtgccagcaggtccaagaccacgcagaattagcgagctgaatatgaaagagaagattgcacccatcccagaagggagtgccttctttatcctgagtaagaccaacccaatcagagtgggatgccacaaactgataaaccatcacatcttcacaaatctgatcctcgtgttcatcatgctctctagcgcagctctggcagctgaagatccaatcaggagccactctttccggaatactattctcgggtacttcgattatgccttcactgccatctttacagtggaaatcctcctgaagatgactacatttggagcctttctccacaagggagccttctgtagaaactactttaacctgctcgacatgctcgtggtgggagtgtctctcgtgtcatttggcattcagtcctccgctatctccgtcgtgaagatcctgagagtcctcagagtgctcaggccactgagagccatcaatagagccaaaggcctcaagcacgtggtccagtgcgtgttcgtggcaatccggaccataggaaacattatgattgtcactacactcctgcagtttatgttcgcctgtattggcgtgcagctgtttaagggaaagttctacaggtgtactgacgaggctaagagcaatcccgaggaatgtagaggtctgttcatcctgtataaggatggcgacgtggactctcccgtcgttcgcgagaggatttggcagaactctgactttaactttgataacgtgctgtccgctatgatggcactgttcacagtgtccactttcgagggctggcctgccctgctctacaaagcaatcgacagtaatggcgagaacataggacctatctacaaccatagggtggaaatctccatattcttcataatctacatcatcattgttgcattcttcatgatgaatatcttcgtgggtttcgtgattgtcactttccaagagcaaggtgagaaggagtataagaattgtgaactcgacaagaatcagaggcagtgtgtggaatacgctctcaaggctaggccactgcgcaggtacatacctaagaatccctaccagtacaagttctggtatgtggtcaacagcagcccattcgagtacatgatgttcgtgctcatcatgctcaatacactgtgcctggctatgcagcattacgagcaatcaaagatgtttaacgacgcaatggacatcctgaatatggtgtttacaggtgtgttcactgtggagatggttctcaaagtcattgccttcaaacccaagggatacttcagtgatgcttggaacactttcgacagcctgattgtcattggaagcatcattgacgtggctctgagcgaagccgacccaactgaatccgagaatgttcctgtgccaacagccactccaggaaattcagaagaatccaaccggataagtatcaccttcttcaggctgttcagggttatgagactggtgaaactcctgagcagaggagagggaattagaacactgctgtggacctttatcaaatccttccaagccctgccatacgtggcactcctgatcgccatgctgttcttcatatacgctgtcattggtatgcagatgtttggcaaagtggcaatgagagataacaatcagatcaatcgcaacaataactttcaaaccttccctcaggctgtcctcctgctgtttcgctgtgctacaggcgaagcatggcaggaaatcatgctcgcatgcctgcctggcaagctgtgcgatcctgaaagtgactataaccctggcgaggagtatacctgtggcagcaacttcgcaatcgtgtacttcatatcattctacatgctgtgcgcattcctgataatcaatctcttcgtggctgtgatcatggacaatttcgattacctgactagagactggagcattctgggtccacatcatctcgatgagttcaagcggatatggtccgaatacgacccagaagccaagggtaggattaagcacctggacgtcgtgaccctgctcagacgcatccagccaccactcgggtttggcaagctgtgtccacacagagtcgcatgcaagaggctcgtcgctatgaacatgccactcaactccgacggcactgttatgttcaatgccacactctttgccctggtgagaacagcactgaagattaagaccgagggaaacctggaacaggccaacgaggaactgagagcagtgataaagaagatttggaagaagacctccatgaaactgctggatcaggtggttcctcctgcaggtgatgacgaagtgacagtgggcaagttctacgctactttcctgattcaggactactttaggaagtttaagaagaggaaggagcaagggctggtgggaaagtatcctgctaagaacacaaccattgccctgcaggcaggactgcggacactgcacgacattggaccagaaattcgcagggcaatctcctgtgacctgcaggacgacgagcctgaggaaaccaagagagaagaagaagatgatgtcttcaaacggaatggagccctgctcgggaatcacgtgaatcatgtgaatagcgacaggagggacagcctgcaacaaacaaataccacccacaggccactgcacgttcaaagaccctcaattccacctgctagtgataccgagaaaccactgttccctccagcaggaaacagcgtctgtcataaccatcataatcacaactcaatagggaagcaagttcccaccagtactaatgccaacctgaacaatgctaacatgtccaaagctgcccatggcaagaggccttctatcggaaatctggaacatgtgtctgagaatggacatcattcttcccacaaacacgacagagaacctcagcggaggtccagcgtgaaacgcactagatactacgagacatacattcggtcagactcaggtgatgagcagctgcctactatttgtagagaagatcctgagattcatgggtacttcagagatcctcactgcctgggagaacaggagtatttcagctcagaggagtgttacgaagacgacagtagcccaacttggagtcgccagaactatggctactacagcaggtatcctggcagaaacatagactctgaaagaccaagaggttaccatcatccacagggtttcctggaggacgatgacagccctgtgtgttatgacagtaggaggtcaccaaggaggagactgctgcctcccactccagcttctcaccgcaggtcatcattcaacttcgaatgtctgagacgccaatccagccaggaggaggtgccttcctctcccatctttccacacaggactgccctgcctctgcacctgatgcagcagcaaatcatggcagtggctggtctggactcctcaaaggctcagaagtacagcccaagtcactccacaagaagctgggcaacacctccagctactcctccatacagggattggacaccttgctatacaccactgattcaagtggagcagagtgaggctctggatcaggttaatgggtcactgccttccctgcatcgctcttcatggtacactgacgaacccgacatctcctaccggaccttcactcctgcaagcctgaccgtgccatctagctttcgcaataagaacagcgacaaacagcgcagtgccgattccctggtggaggctgttctcatctccgagggtctgggaagatatgccagggacccaaagtttgtctctgctaccaagcacgaaattgccgatgcatgcgacctcaccatagatgaaatggagagcgctgcaagtacactgctgaacgggaatgttagaccaagagccaacggagatgtgggacctctctctcatagacaggattatgaactgcaagatttcggacctgggtattctgacgaggaaccagacccaggacgggacgaagaggacctcgcagatgaaatgatatgcataacaaccctg

SEQ ID NO: 3 - DNA, 1452 bp, Artificial Sequence
  Synthetic
atgtatgatgattcctatgtgccaggtttcgaagactctgaggctggttctgcagatagctacacctctaggccaagcctggattccgacgtctccctggaggaggatcgcgaatccgctaggagagaagtcgagtctcaggctcaacagcagctggagagggctaagcataagcctgtggctttcgcagtgagaactaacgtgtcctactgcggagttctcgacgaagaatgtcctgttcagggatctggagtgaacttcgaggccaaggatttcctgcacatcaaggagaagtactctaacgattggtggatcggcaggctggtgaaagagggaggtgatatcgccttcatcccttctccacagaggctcgaatccattcgcctgaaacaggagcagaaagctagacgcagcggaaacccaagctctctgtccgacatcggcaataggagaagccctccaccttctctggctaagcagaagcagaaacaggctgaacacgtgcctccatacgacgttgtcccatccatgaggccagtggtgctggtgggaccttccctcaaaggctatgaagttaccgacatgatgcagaaggcactcttcgatttcctcaagcatagatttgacggtcgcatctctatcactagagtgactgccgatctctctctcgccaagcgctctgtcctgaacaatcctggaaagcgcactatcatcgaacgcagcagcgctaggtcctccattgccgaggtgcaatctgaaatcgagcgcatctttgagctggccaagagcctgcaactggtggtgctggacgcagacactatcaaccacccagctcaactcgcaaagacctccctcgcacccatcattgtgtttgttaaggtgagcagcccaaaggttctgcagaggctgattaggagcagaggcaaatctcagatgaagcacctcacagtgcagatgatggcctacgataagctggttcagtgcccacccgagtctttcgatgtgattctcgatgagaaccagctggaggatgcttgcgagcacctcgcagagtacctcgaagtctactggagagccactcatcatccagcaccaggacctggactgctcggaccaccttccgctatcccaggactgcagaaccagcagctcctgggtgaaagaggcgaggaacatagcccactcgaaagagactctctcatgccctctgatgaggcttccgagtccagcagacaggcatggaccggttcttcccagcgctccagcaggcacctcgaagaggattacgcagacgcataccaggacctgtaccagccacacagacagcacactagcggtctgcccagcgctaatggtcacgatccacaagatagactgctggctcaggactccgagcacaaccatagcgaccgcaactggcaacgcaatagaccttggccaaaggattcctac

SEQ ID NO: 4 - DNA, 1815 bp, Artificial Sequence
  Synthetic
atgcaatgttgtggactggttcatagacgcagagttagagtgtcttacggctccgctgactcctacacaagcaggccatctgacagcgatgtctccctggaagaagaccgggaggcagtgagacgcgaggctgagcggcaggctcaggcccagctggaaaaggcaaagaccaaaccagtggcttttgccgtgcggactaacgtgagctatagcgcagctcacgaagatgacgtcccagtccccggcatggcaattagcttcgaggcaaaggactttctgcatgtgaaggagaagttcaacaatgactggtggatcgggcgcctggtcaaggaaggctgtgagatcggctttattccttccccagtcaaactcgaaaacatgagactgcaacacgagcaaagggccaaacaaggaaagttttattccagcaagagcggcggcaacagctccagcagcctcggagatatcgtgcctagcagcaggaagtccacacctcccagcagcgcaattgacatcgatgctactgggctggatgcagaagagaacgacatcccagcaaatcaccggtcccctaaaccctctgccaacagcgtgacctctccccatagcaaagagaagcggatgcctttcttcaagaagaccgagcacacccctccctacgacgtcgtcccttctatgcggccagttgttctggttggaccaagcctgaaagggtacgaagtgaccgatatgatgcaaaaagccctgtttgactttctcaaacaccgctttgagggcagaatctccattacacgggtcaccgcagatattagcctcgccaagagatccgtcctgaataatccttccaaacacgccatcatagagaggtccaacaccaggtcttccctggccgaggtgcagtccgagattgagcgcattttcgaactcgctaggaccctgcaactggttgtgctggatgccgataccattaatcaccccgcccagctgtctaaaactagcctggctcctattatagtgtacgttaagatatcatcaccaaaggttctgcagagactgatcaaatctaggggcaagagccaagctaagcacctcaacgtgcagatggtggccgcagataaactcgcacagtgccccccagaactgttcgacgtcatcctggatgagaaccagctcgaagacgcttgtgaacatctcgcagactacctggaggcatattggaaggctacccaccccccttccagctcactgcctaatccactgctgtcaaggacactggctacatcatccctgccactcagccccaccctcgcaagcaattcccagggatctcagggggaccagaggactgatcgctccgcccccatcaggtcagcttcccaggccgaagaagaacctagcgtggagccagtgaagaaaagccagcatagatcatcttctagcgccccccaccataaccatcgctcaggcacatcccggggactctctcgccaggagacttttgattccgagacacaggaatccagggattcagcctatgtggagcccaaggaggattattcccatgaccatgtggatcactatgcctctcaccgggaccacaatcaccgcgacgaaactcacggttcttctgatcacagacaccgggagagccggcatcggtctagggacgtcgatcgcgaacaagaccataatgagtgcaacaagcaacggagccgccataaaagcaaggacagatactgtgagaaagacggcgaagtcatcagcaagaaaagaaacgaagccggggagtggaacagagacgtctacatccggcaa

SEQ ID NO: 5 - DNA, 3273 bp, Artificial Sequence
  Synthetic
atggctgctggatgtctcctcgctctgactctcactctctttcaaagcctgctgattggaccatcctccgaggaacctttcccttctgctgtgaccatcaagtcctgggtcgataagatgcaggaggacctggtgaccctggctaagactgccagcggagtgaaccaactcgtcgatatctacgagaagtaccaagacctctacaccgtcgagccaaacaatgctcgccaactggtggagattgctgccagggatatcgagaagctcctcagcaatcgctctaaagcactggtcaggctcgctctcgaagctgagaaggtgcaggcagcccaccagtggagagaggacttcgcatctaacgaagtggtttactacaacgccaaagacgacctggacccagagaagaatgattctgaacctggctcccagaggatcaaacctgtcttcattgaagacgctaactttggacgccagattagctaccagcacgctgcagtccatatcccaactgacatctatgagggcagcactatcgttctcaatgaactgaattggacctctgcactggacgaagtgttcaagaagaatagggaggaggacccttctctgctgtggcaggtgttcggctccgcaacaggactcgctcgctactaccctgcttctccttgggtggataacagcaggactcccaacaagatcgacctgtacgatgtgagacgcagaccttggtacattcagggagcagccagccctaaagacatgctcatcctggtcgatgtttctggctccgtttctggtctgacactgaagctcatcagaacatctgtgtccgagatgctggagactctgtccgacgatgactttgtcaacgttgccagcttcaactccaacgcacaagatgtgtcctgctttcagcacctggtgcaagctaacgtcaggaacaagaaagtcctgaaggatgccgtgaacaacattaccgctaagggcattacagactacaagaaaggcttctctttcgccttcgagcagctcctgaactacaacgttagcagggcaaactgcaacaagatcatcatgctgtttacagatggtggcgaagagagagcacaggaaatcttcaacaagtacaacaaggacaagaaggtgagggtcttcacattctctgttggccagcataactacgacagaggtccaatccagtggatggcttgcgagaacaagggttactactacgagattccctctatcggtgcaattcgcatcaacacacaggaatacctggatgtcctgggtagacctatggtgctggctggtgacaaagctaagcaagtccagtggactaacgtgtatctcgacgctctcgagctcggactcgtgatcacaggtacactccctgtgttcaacattactggccagtttgagaacaagacaaacctcaagaatcaactcatcctgggtgtcatgggtgtggacgtcagcctggaggatatcaagagactcacacctcgctttacactgtgccctaatggctactactttgccatcgacccaaacggatacgtcctgctccatccaaacctccagcccaagaatcccaagtctcaagaacctgtgaccctcgactttctggacgcagagctggagaacgacatcaaagtggagatcagaaacaagatgatcgacggtgaatctggagagaagacattccgcaccctggtgaaatcccaggatgaacgctacatcgacaagggcaacaggacctacacttggactccagtcaacggcactgactattctctggcactcgtgctgcccacatactctttctactacatcaaggctaagctggaggaaaccattacccaggctaggagcaagaaaggcaagatgaaggactctgaaaccctgaagcctgacaactttgaggaatccggttacaccttcatcgctcccagagactattgcaacgacctcaagatttccgacaacaacacagagttcctgctcaacttcaatgagttcattgataggaagactcccaacaatccctcctgtaatgctgatctcatcaacagagtgctgctcgacgctggctttaccaacgagctggtccagaactattggtccaagcagaagaacatcaaaggcgtcaaggccagattcgtggtgactgatggaggcattactagggtttacccaaaggaagctggagagaattggcaagagaaccctgagacatacgaggatagcttctacaagagatccctggacaatgataactacgtgtttacagcaccttacttcaacaaatctggtccaggtgcttacgaaagcggaatcatggtgtccaaagccgtcgaaatctacatccaaggaaagctcctcaagccagcagtcgtgggcatcaagatcgacgtgaatagctggatcgagaacttcacaaagacatccattcgcgacccttgcgctggtcccgtgtgcgactgtaagcgcaactctgacgttatggactgtgttatcctcgacgacggaggattcctcctcatggctaatcatgacgactacactaatcagattggaagattcttcggtgagattgatccttccctcatgaggcacctggtcaacatctccgtgtacgctttcaacaagtcttacgactatcagtctgtgtgtgaacctggagcagctcctaaacagggtgctggtcaccgctctgcttatgtcccttccgttgccgacatcctgcagatcggctggtgggcaactgctgctgcttggtctattctgcagcagttcctgctgtctctgacatttcctcgcctcctggaagccgtcgagatggaagacgacgactttactgcaagcctgagcaagcagagctgtatcaccgaacagacccagtacttcttcgataacgactccaagtccttctctggtgtgctcgattgtggcaattgtagcagaatcttccacggtgagaaactgatgaacacaaatctgatcttcatcatggtggaatccaagggcacctgcccttgcgacacaagactgctgatccaagccgagcagacttctgatggacctaacccttgcgacatggtcaagcagccacgctataggaaaggacccgacgtctgctttgataacaacgtgctggaggattacactgactgtggaggcgtgtccggactcaacccaagcctgtggtacatcattggtatccagttcctgctcctctggctggtttccggttccacccacaggctgctg

SEQ ID NO: 6 - DNA, 513 bp, Artificial Sequence
  Synthetic
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 7 - DNA, 669 bp, Artificial Sequence
  Synthetic
atgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaa

SEQ ID NO: 8 - DNA, 1248 bp, Artificial Sequence
  Synthetic
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaa

SEQ ID NO: 9 - DNA, 57 bp, Artificial Sequence
  Synthetic
gctactaacttcagcctgctgaagcaggctggagacgtggaggagaaccctggacct

SEQ ID NO: 10 - DNA, 561 bp, Artificial Sequence
  Synthetic
atgactaacaaatgtctcctccagattgcactgctgctgtgtttctctaccactgctctctccatgagctacaacctgctgggcttcctccaacgctcctccaactttcaatgtcagaaactcctgtggcagctgaatggtcgcctcgaatactgcctcaaggataggatgaacttcgacatccctgaggaaatcaaacagctccagcaattccagaaggaagatgcagctctgaccatctatgagatgctccagaacatcttcgctatctttagacaagactcctccagcacaggatggaatgagacaatcgttgagaacctgctcgctaacgtctaccatcagatcaaccacctgaagaccgtgctggaggagaagctggagaaggaagactttaccagaggcaagctgatgagcagcctgcacctgaagaggtactacggcagaatcctgcactatctgaaggccaaggaatactcccattgcgcatggaccattgtcagagtggagatcctccgcaacttctacttcatcaacaggctgactggctacctgagaaac

SEQ ID NO: 11 - DNA, 8024 bp, Artificial Sequence
  Synthetic
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagtttaaacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagctcgagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctgacggcaataaaaagacagaataaaacgcacggtgttgggtcgtttgttcataagcggccgcgtaagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 12 - DNA, 15051 bp, Artificial Sequence
  Synthetic
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagtttaaacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagctcgagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctgacggcaataaaaagacagaataaaacgcacggtgttgggtcgtttgttcataagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctaccggtgccaccatggtgaacgagaacacaaggatgtatatcccagaagagaatcatcagggctccaactacggttctccaagacctgcacacgccaacatgaacgctaacgcagctgctggactggcaccagaacacatccctactccaggagctgcactgtcctggcaggctgctatcgatgctgctaggcaggctaagctgatgggctcagctggcaatgcaaccatcagtactgtttccagtacccagcgcaaacgccagcagtatgggaagccaaagaagcagggctccaccactgcaactagaccacctagggcactcctctgtctcaccctcaagaatcctattcgcagggcttgcatttctatcgtggagtggaaacccttcgaaatcatcattctgctgaccatctttgctaactgtgtcgctctcgctatctatatcccatttcctgaggacgattctaatgcaaccaactctaatctggagagagtggagtatctcttcctcatcatcttcacagtggaagccttcctgaaagtgattgcctatggcctcctctttcatccaaacgcttacctgcgcaacggatggaacctgctcgacttcatcattgtggttgtcggactgtttagtgccattctggagcaggccacaaaggctgacggagccaatgctctgggtgggaagggtgctggctttgatgtcaaggctctgagagctttcagagtcctgaggcctctgaggctcgtgtctggcgtcccaagcctgcaggtcgtgctgaactctatcatcaaagccatggtccctctgctccacattgctctcctggtcctgttcgtgatcattatctacgcaatcatcggtctggaactgtttatgggaaagatgcataagacatgctacaaccaggaagggattgccgatgtgcctgcagaggacgatcctagcccatgcgcactggaaactgggcacggacggcagtgccagaacggaactgtgtgcaaacctggctgggacggtccaaagcatggcattactaactttgataacttcgccttcgccatgctcacagtcttccagtgcattactatggaaggatggacagacgtcctgtactgggtgaacgatgctgttggacgggattggccttggatctacttcgtgaccctgatcatcattgggtcattctttgtcctgaacctcgtgctcggagtcctcagtggagagttctccaaggagagggagaaggccaaagccagaggagacttccagaagctcagggagaaacagcagctcgaagaggatctcaagggatacctcgattggatcacacaagcagaggacatagatccagagaacgaggatgagggaatggacgaagagaaaccacggaatatgtctatgcccacatcagaaaccgaaagtgtgaataccgagaacgtggcaggtggcgacatcgaaggtgagaactgtggagcacggctggcacacagaatatctaagagcaaattctcaaggtactggagaaggtggaatagattctgcaggaggaagtgtagagctgcagttaagtctaacgtgttctactggctcgtgatctttctcgtgttcctgaatactctcaccatagccagcgagcactacaaccagcccaattggctgaccgaggtccaagacaccgctaacaaggctctcctggcactgttcacagctgaaatgctgctgaagatgtatagcctgggtctgcaggcatacttcgtctccctcttcaatcggtttgactgctttgtcgtgtgtggtggaatcctcgaaaccatcctggtggaaaccaagatcatgtcaccactcgggatctctgttctccgctgtgttcgcctgctgcggatcttcaagatcactcggtattggaactccctctctaatctggtggccagtctcctcaatagtgtgaggtccattgcttctctgctcctcctgctgttcctgttcattatcatctttagtctgctgggaatgcagctcttcggtgggaaattcaacttcgatgagatgcagacaagacactacccttatgatgtccctgattatgctgtcacctttgatgaaatgcagaccaggagaagcacatttgataactttccacagagtctgctgaccgtgttccagattctgacaggagaagactggaactcagtgatgtacgacggcatcatggcatacggaggtccctcatttcctggaatgctggtttgtatctacttcatcatcctgtttatctgtgggaattacatcctgctcaatgtgttcctggccattgccgtggacaacctggctgacgctgaaagcctgacctccgctcagaaggaggaagaggaggagaaagaaaggaagaagctggcacggacagccagtccagagaagaagcaagaactcgtggagaaaccagcagtgggagaaagcaaggaggagaagatcgaactcaaatccatcacagccgatggcgagagcccacctgccactaagattaacatggacgatctccagccaaacgagaatgaggataagtctccatatccaaatcccgagacaactggcgaggaggacgaagaggagcctgagatgcctgttggacccaggcctagacctctctcagagctgcacctgaaagagaaagccgtgcccatgccagaggcttctgctttcttcatcttctccagcaacaatcgctttcggctgcaatgccaccgcatcgtcaatgacacaatcttcaccaacctcattctgttctttatcctcctctcttccatcagtctcgctgctgaagaccctgtccagcatacatccttccggaatcacatcctgggcaatgccgactacgtgttcactagcatctttacactggagattatcctgaagatgacagcctacggtgccttcctgcacaaagggtccttctgcaggaactacttcaatatcctggatctgctggtcgtctctgtgtctctcattagctttggtatacaaagttcagcaatcaacgtcgtcaagattctgagggtgctgagagttctgaggccactgagggcaatcaatagagccaaaggactcaagcacgtggtccagtgcgtgttcgttgcaatccggaccattggaaacatcgtgatcgtgactactctgctgcagtttatgttcgcatgtattggagtgcagctcttcaagggaaagctgtatacctgctctgacagttccaagcagaccgaggcagaatgcaagggtaactacatcacttacaaggatggcgaagtggatcatccaatcatacagccacgcagttgggagaacagcaagtttgacttcgacaacgtgctggctgccatgatggctctcttcaccgtgtccacattcgaaggatggcctgaactcctctatagaagcatcgattctcatactgaggacaagggacccatctataactatcgggtcgagatttcaatcttcttcatcatatacatcatcatcatcgccttcttcatgatgaacatcttcgtgggcttcgtcatcgttacattccaggagcaaggcgagcaggagtacaagaattgcgaactcgacaagaaccagaggcagtgtgtggaatatgccctcaaagcaaggcctctcagacggtacatcccaaagaaccaacatcaatacaaagtctggtacgtggtcaactccacctactttgagtacctgatgtttgtcctgatactgctcaatacaatatgtctcgccatgcaacactatgggcaaagttgtctgttcaagatagcaatgaacatcctgaatatgctgtttactggcctgttcaccgtggaaatgatcctgaagctgatcgctttcaagcctaagcattacttctgcgacgcctggaatactttcgacgctctcatcgtcgttggatcaatcgtggacatagccatcacagaagtgaacccagccgagcacacacagtgcagcccatccatgaacgctgaagagaatagcaggataagcatcacattctttagactctttcgcgtgatgagactggtcaagctgctgtccagaggagagggcataaggaccctgctctggacattcataaagagcttccaagcactgccctatgttgctctgctgatcgtgatgctgttcttcatctacgcagtcatcggcatgcaagtgttcggcaagatcgcactgaatgacaccaccgagatcaatagaaacaacaacttccagacctttcctcaggctgtgctgctgctgtttcggtgtgctactggagaggcttggcaggacatcatgctcgcatgcatgccagggaagaaatgcgcaccagagagtgaacctagcaactctaccgaaggcgaaacaccctgcggttcaagtttcgctgtcttctacttcattagcttctacatgctctgcgcatttctgatcataaacctctttgttgctgtcatcatggacaatttcgactacctgactagagattggtccatcctgggtccacatcacctggacgagttcaaacgcatctgggctgagtacgacccagaggccaaaggacgcatcaagcatctggacgtggtgacactgctcagacggattcaaccaccactgggctttggaaagctctgccctcatcgggtcgcttgcaaacgcctggtgagcatgaatatgcctctcaatagtgatgggaccgttatgtttaacgcaacactgttcgctctggtgcgcacagccctcagaatcaagactgaaggaaacctggagcaagccaatgaagaactcagagccattatcaagaagatttggaagaggactagcatgaagctgctggatcaggttgttcctccagctggcgacgatgaagtcaccgtgggcaagttctatgcaacatttctcattcaggaatactttcggaaattcaagaagaggaaggagcaaggactcgtcgggaaacccagccagcgcaatgccctcagtctgcaagctgggctcagaacactgcatgacattggtcctgagatacgcagagccatctctggcgatctgacagctgaagaggagctggacaaagcaatgaaggaagccgtgtccgctgcctccgaggacgacatattccgcagagctggtggcctcttcgggaaccacgttagctattaccagtccgatggaagaagtgctttcccacagaccttcaccactcagaggccactgcatatcaataaggctggcagcagccaaggcgatacagagagtccatcccatgagaagctggtggatagtaccttcactcctagtagctacagtagcacaggcagcaacgccaatatcaacaatgccaacaacaccgctctcgggaggctcccaagaccagcaggataccctagcaccgtctccaccgtggagggacatggaccacctctgagcccagccattagggtgcaagaagtggcctggaagctgtcttctaaccgctgccatagtcgcgagtcccaggcagccatggctggccaggaagaaacatcacaggacgaaacttatgaagtgaagatgaaccacgataccgaggcttgttccgagccctcactcctgagcaccgagatgctgtcataccaggatgatgagaataggcagctgaccctcccagaggaagacaaacgggacatcagacagagcccaaagagaggtttcctgagaagcgcttcactgggcaggagagccagctttcatctcgaatgtctgaaacgccagaaagacagaggtggcgacattagccagaagacagtgctgcctctgcacctggtgcaccatcaggcactggcagtcgctgggctgagcccactgctgcagaggtcacatagtcctgcctctttccctagacctttcgccactccaccagccactcctggaagcagaggctggccaccacagccagttcccactctcagactcgaaggtgtggaatcatccgagaagctcaatagctcattcccaagcatccattgtggctcttgggcagagacaactcctggaggtggtggaagctctgctgctaggagagttaggccagtttcactgatggtgccctcccaggctggtgcaccaggacggcagtttcacggatctgcatcctctctggtggaggcagtcctcattagcgaagggctgggacagtttgcccaggacccaaagttcattgaagtgacaacccaagagctggcagacgcctgtgacatgacaatagaagaaatggaatcagctgctgacaacattctgagtggaggagcaccacaaagtccaaatggagcactgctgcccttcgtcaactgtagggatgctggtcaggatagagcaggtggcgaggaggatgcaggatgtgttagggctagaggtagaccatctgaagaggaactgcaggactccagagtgtacgtgagcagcctgtaagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 13 - DNA, 15120 bp, Artificial Sequence
  Synthetic
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagtttaaacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagctcgagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctgacggcaataaaaagacagaataaaacgcacggtgttgggtcgtttgttcataagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctaccggtgccaccatgatgatgatgatgatgatgaagaagatgcagcaccaaaggcagcaacaagcagatcacgcaaatgaggccaattacgccagagggaccaggctccctctctcaggagaaggtcctactagccagccaaactctagcaagcaaacagtgctgagttggcaggctgcaattgacgcagctagacaagccaaagcagcccagacaatgagtacatctgcaccacctcccgtcgggagtctgagccaaagaaagaggcagcagtatgccaagagcaagaagcagggtaatagcagtaacagcaggccagctagggcactcttctgtctgagcctgaataaccctattaggagggcttgtatcagtatcgtggagtggaagccattcgacatcttcatcctcctggctatcttcgccaactgtgtggcactcgcaatctacatacctttccctgaggacgactccaattccaccaaccacaacctggagaaagtcgaatacgccttcctcatcatattcactgtcgagacattcctgaagatcatcgcttacggactgctgctccatcccaacgcatacgtccggaacgggtggaacctgctcgacttcgtcattgtgatcgtgggactgttctccgtcatcctcgaacagctcactaaagagacagagggtggcaaccattcttctggaaagagcggaggattcgatgtcaaagctctgagagctttcagagtgctcagacctctgagactggtgtcaggagtgccatccctgcaggtggtgctgaactctatcatcaaagcaatggttccactcctgcacatagccctgctggttctgtttgtcatcatcatctacgccatcatcggtctggagctgttcataggcaagatgcacaagacctgcttctttgccgattcagatattgtggcagaagaggacccagctccatgtgcctttagtggtaatggacgccagtgcactgccaacggcactgaatgtcggtctggatgggtgggacctaatggaggcatcacaaactttgacaactttgcctttgccatgctcacagtgttccagtgcattaccatggagggctggacagacgtgctgtactggatgaatgacgcaatgggcttcgaactgccttgggtgtactttgtgtctctggttatctttggaagtttctttgtgctgaatctggttctcggagtgctgtctggagagttctctaaggaaagagagaaggccaaggctagaggcgacttccagaagctcagggagaagcagcagctggaggaggatctcaaagggtatctggattggataacccaagccgaggatattgaccctgagaacgaggaggagggaggcgaggaaggcaagagaaacacatcaatgccaaccagtgagactgagtctgtcaacaccgagaatgtctctggagagggtgagaatagaggatgttgtggctccctgtgccaagccatttctaagtctaagctgagcagaaggtggaggaggtggaatcggtttaaccgcagacggtgtagagctgctgttaagtcagttactttctattggctggtgatcgtgctggtctttctgaacaccctgacaatctcctctgaacactataaccagcctgactggctgacccagatacaggacattgccaacaaggtgctgctcgctctctttacttgcgaaatgctggtgaagatgtactcactcggactgcaggcctacttcgttagcctcttcaacagatttgattgctttgtcgtgtgtggaggaatcacagaaaccatcctcgtggaactggagatcatgagtcctctgggtataagcgtgttcaggtgcgtgagactgctgagaatcttcaaggtgactcggcactggacatctctctcaaacctcgtggcttccctcctgaacagtatgaaatccattgccagcctgctcctgctcctgttcctgttcatcatcatcttctccctgctcgggatgcagctgttcggtggaaagtttaactttgatgaaacccagacaagacactacccttatgatgtccctgattatgctgtcacctttgatgaaatgcagactaagaggagcacatttgacaatttcccacaagccctgctcactgtgtttcagatcctgacaggtgaagattggaacgctgtgatgtacgatgggatcatggcatacggaggtcctagtagctcagggatgattgtgtgcatctacttcatcatcctgtttatctgcggaaactacatcctcctgaatgtcttcctcgcaattgcagtggataatctggcagatgccgaatctctgaataccgcacagaaagaagaagctgaagagaaggagaggaagaagatcgctcggaaagaatctctggagaataagaagaacaacaagccagaggtgaatcagattgctaactcagacaacaaggtcaccatcgacgattatcgcgaggaggatgaggacaaggacccatatccaccctgcgacgtgcctgtcggagaagaagaagaggaagaggaggaggacgagccagaggtgccagcaggtccaagaccacgcagaattagcgagctgaatatgaaagagaagattgcacccatcccagaagggagtgccttctttatcctgagtaagaccaacccaatcagagtgggatgccacaaactgataaaccatcacatcttcacaaatctgatcctcgtgttcatcatgctctctagcgcagctctggcagctgaagatccaatcaggagccactctttccggaatactattctcgggtacttcgattatgccttcactgccatctttacagtggaaatcctcctgaagatgactacatttggagcctttctccacaagggagccttctgtagaaactactttaacctgctcgacatgctcgtggtgggagtgtctctcgtgtcatttggcattcagtcctccgctatctccgtcgtgaagatcctgagagtcctcagagtgctcaggccactgagagccatcaatagagccaaaggcctcaagcacgtggtccagtgcgtgttcgtggcaatccggaccataggaaacattatgattgtcactacactcctgcagtttatgttcgcctgtattggcgtgcagctgtttaagggaaagttctacaggtgtactgacgaggctaagagcaatcccgaggaatgtagaggtctgttcatcctgtataaggatggcgacgtggactctcccgtcgttcgcgagaggatttggcagaactctgactttaactttgataacgtgctgtccgctatgatggcactgttcacagtgtccactttcgagggctggcctgccctgctctacaaagcaatcgacagtaatggcgagaacataggacctatctacaaccatagggtggaaatctccatattcttcataatctacatcatcattgttgcattcttcatgatgaatatcttcgtgggtttcgtgattgtcactttccaagagcaaggtgagaaggagtataagaattgtgaactcgacaagaatcagaggcagtgtgtggaatacgctctcaaggctaggccactgcgcaggtacatacctaagaatccctaccagtacaagttctggtatgtggtcaacagcagcccattcgagtacatgatgttcgtgctcatcatgctcaatacactgtgcctggctatgcagcattacgagcaatcaaagatgtttaacgacgcaatggacatcctgaatatggtgtttacaggtgtgttcactgtggagatggttctcaaagtcattgccttcaaacccaagggatacttcagtgatgcttggaacactttcgacagcctgattgtcattggaagcatcattgacgtggctctgagcgaagccgacccaactgaatccgagaatgttcctgtgccaacagccactccaggaaattcagaagaatccaaccggataagtatcaccttcttcaggctgttcagggttatgagactggtgaaactcctgagcagaggagagggaattagaacactgctgtggacctttatcaaatccttccaagccctgccatacgtggcactcctgatcgccatgctgttcttcatatacgctgtcattggtatgcagatgtttggcaaagtggcaatgagagataacaatcagatcaatcgcaacaataactttcaaaccttccctcaggctgtcctcctgctgtttcgctgtgctacaggcgaagcatggcaggaaatcatgctcgcatgcctgcctggcaagctgtgcgatcctgaaagtgactataaccctggcgaggagtatacctgtggcagcaacttcgcaatcgtgtacttcatatcattctacatgctgtgcgcattcctgataatcaatctcttcgtggctgtgatcatggacaatttcgattacctgactagagactggagcattctgggtccacatcatctcgatgagttcaagcggatatggtccgaatacgacccagaagccaagggtaggattaagcacctggacgtcgtgaccctgctcagacgcatccagccaccactcgggtttggcaagctgtgtccacacagagtcgcatgcaagaggctcgtcgctatgaacatgccactcaactccgacggcactgttatgttcaatgccacactctttgccctggtgagaacagcactgaagattaagaccgagggaaacctggaacaggccaacgaggaactgagagcagtgataaagaagatttggaagaagacctccatgaaactgctggatcaggtggttcctcctgcaggtgatgacgaagtgacagtgggcaagttctacgctactttcctgattcaggactactttaggaagtttaagaagaggaaggagcaagggctggtgggaaagtatcctgctaagaacacaaccattgccctgcaggcaggactgcggacactgcacgacattggaccagaaattcgcagggcaatctcctgtgacctgcaggacgacgagcctgaggaaaccaagagagaagaagaagatgatgtcttcaaacggaatggagccctgctcgggaatcacgtgaatcatgtgaatagcgacaggagggacagcctgcaacaaacaaataccacccacaggccactgcacgttcaaagaccctcaattccacctgctagtgataccgagaaaccactgttccctccagcaggaaacagcgtctgtcataaccatcataatcacaactcaatagggaagcaagttcccaccagtactaatgccaacctgaacaatgctaacatgtccaaagctgcccatggcaagaggccttctatcggaaatctggaacatgtgtctgagaatggacatcattcttcccacaaacacgacagagaacctcagcggaggtccagcgtgaaacgcactagatactacgagacatacattcggtcagactcaggtgatgagcagctgcctactatttgtagagaagatcctgagattcatgggtacttcagagatcctcactgcctgggagaacaggagtatttcagctcagaggagtgttacgaagacgacagtagcccaacttggagtcgccagaactatggctactacagcaggtatcctggcagaaacatagactctgaaagaccaagaggttaccatcatccacagggtttcctggaggacgatgacagccctgtgtgttatgacagtaggaggtcaccaaggaggagactgctgcctcccactccagcttctcaccgcaggtcatcattcaacttcgaatgtctgagacgccaatccagccaggaggaggtgccttcctctcccatctttccacacaggactgccctgcctctgcacctgatgcagcagcaaatcatggcagtggctggtctggactcctcaaaggctcagaagtacagcccaagtcactccacaagaagctgggcaacacctccagctactcctccatacagggattggacaccttgctatacaccactgattcaagtggagcagagtgaggctctggatcaggttaatgggtcactgccttccctgcatcgctcttcatggtacactgacgaacccgacatctcctaccggaccttcactcctgcaagcctgaccgtgccatctagctttcgcaataagaacagcgacaaacagcgcagtgccgattccctggtggaggctgttctcatctccgagggtctgggaagatatgccagggacccaaagtttgtctctgctaccaagcacgaaattgccgatgcatgcgacctcaccatagatgaaatggagagcgctgcaagtacactgctgaacgggaatgttagaccaagagccaacggagatgtgggacctctctctcatagacaggattatgaactgcaagatttcggacctgggtattctgacgaggaaccagacccaggacgggacgaagaggacctcgcagatgaaatgatatgcataacaaccctgtaagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 14 - DNA, 14367 bp, Artificial Sequence
  Synthetic
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatgactaacaaatgtctcctccagattgcactgctgctgtgtttctctaccactgctctctccatgagctacaacctgctgggcttcctccaacgctcctccaactttcaatgtcagaaactcctgtggcagctgaatggtcgcctcgaatactgcctcaaggataggatgaacttcgacatccctgaggaaatcaaacagctccagcaattccagaaggaagatgcagctctgaccatctatgagatgctccagaacatcttcgctatctttagacaagactcctccagcacaggatggaatgagacaatcgttgagaacctgctcgctaacgtctaccatcagatcaaccacctgaagaccgtgctggaggagaagctggagaaggaagactttaccagaggcaagctgatgagcagcctgcacctgaagaggtactacggcagaatcctgcactatctgaaggccaaggaatactcccattgcgcatggaccattgtcagagtggagatcctccgcaacttctacttcatcaacaggctgactggctacctgagaaactaagtttaaacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatacatagcagcaattggcaagctgcttatatagaacttgcggcgattggcatgccgctttaaaattttattttattttcttttcttttccgaatcggatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagctcgagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctgacggcaataaaaagacagaataaaacgcacggtgttgggtcgtttgttcataagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctaccggtgccaccatggtgaacgagaacacaaggatgtatatcccagaagagaatcatcagggctccaactacggttctccaagacctgcacacgccaacatgaacgctaacgcagctgctggactggcaccagaacacatccctactccaggagctgcactgtcctggcaggctgctatcgatgctgctaggcaggctaagctgatgggctcagctggcaatgcaaccatcagtactgtttccagtacccagcgcaaacgccagcagtatgggaagccaaagaagcagggctccaccactgcaactagaccacctagggcactcctctgtctcaccctcaagaatcctattcgcagggcttgcatttctatcgtggagtggaaacccttcgaaatcatcattctgctgaccatctttgctaactgtgtcgctctcgctatctatatcccatttcctgaggacgattctaatgcaaccaactctaatctggagagagtggagtatctcttcctcatcatcttcacagtggaagccttcctgaaagtgattgcctatggcctcctctttcatccaaacgcttacctgcgcaacggatggaacctgctcgacttcatcattgtggttgtcggactgtttagtgccattctggagcaggccacaaaggctgacggagccaatgctctgggtgggaagggtgctggctttgatgtcaaggctctgagagctttcagagtcctgaggcctctgaggctcgtgtctggcgtcccaagcctgcaggtcgtgctgaactctatcatcaaagccatggtccctctgctccacattgctctcctggtcctgttcgtgatcattatctacgcaatcatcggtctggaactgtttatgggaaagatgcataagacatgctacaaccaggaagggattgccgatgtgcctgcagaggacgatcctagcccatgcgcactggaaactgggcacggacggcagtgccagaacggaactgtgtgcaaacctggctgggacggtccaaagcatggcattactaactttgataacttcgccttcgccatgctcacagtcttccagtgcattactatggaaggatggacagacgtcctgtactgggtgaacgatgctgttggacgggattggccttggatctacttcgtgaccctgatcatcattgggtcattctttgtcctgaacctcgtgctcggagtcctcagtggagagttctccaaggagagggagaaggccaaagccagaggagacttccagaagctcagggagaaacagcagctcgaagaggatctcaagggatacctcgattggatcacacaagcagaggacatagatccagagaacgaggatgagggaatggacgaagagaaaccacggaatatgtctatgcccacatcagaaaccgaaagtgtgaataccgagaacgtggcaggtggcgacatcgaaggtgagaactgtggagcacggctggcacacagaatatctaagagcaaattctcaaggtactggagaaggtggaatagattctgcaggaggaagtgtagagctgcagttaagtctaacgtgttctactggctcgtgatctttctcgtgttcctgaatactctcaccatagccagcgagcactacaaccagcccaattggctgaccgaggtccaagacaccgctaacaaggctctcctggcactgttcacagctgaaatgctgctgaagatgtatagcctgggtctgcaggcatacttcgtctccctcttcaatcggtttgactgctttgtcgtgtgtggtggaatcctcgaaaccatcctggtggaaaccaagatcatgtcaccactcgggatctctgttctccgctgtgttcgcctgctgcggatcttcaagatcactcggtattggaactccctctctaatctggtggccagtctcctcaatagtgtgaggtccattgcttctctgctcctcctgctgttcctgttcattatcatctttagtctgctgggaatgcagctcttcggtgggaaattcaacttcgatgagatgcagacaagacactacccttatgatgtccctgattatgctgtcacctttgatgaaatgcagaccaggagaagcacatttgataactttccacagagtctgctgaccgtgttccagattctgacaggagaagactggaactcagtgatgtacgacggcatcatggcatacggaggtccctcatttcctggaatgctggtttgtatctacttcatcatcctgtttatctgtgggaattacatcctgctcaatgtgttcctggccattgccgtggacaacctggctgacgctgaaagcctgacctccgctcagaaggaggaagaggaggagaaagaaaggaagaagctggcacggacagccagtccagagaagaagcaagaactcgtggagaaaccagcagtgggagaaagcaaggaggagaagatcgaactcaaatccatcacagccgatggcgagagcccacctgccactaagattaacatggacgatctccagccaaacgagaatgaggataagtctccatatccaaatcccgagacaactggcgaggaggacgaagaggagcctgagatgcctgttggacccaggcctagacctctctcagagctgcacctgaaagagaaagccgtgcccatgccagaggcttctgctttcttcatcttctccagcaacaatcgctttcggctgcaatgccaccgcatcgtcaatgacacaatcttcaccaacctcattctgttctttatcctcctctcttccatcagtctcgctgctgaagaccctgtccagcatacatccttccggaatcacatcctgggcaatgccgactacgtgttcactagcatctttacactggagattatcctgaagatgacagcctacggtgccttcctgcacaaagggtccttctgcaggaactacttcaatatcctggatctgctggtcgtctctgtgtctctcattagctttggtatacaaagttcagcaatcaacgtcgtcaagattctgagggtgctgagagttctgaggccactgagggcaatcaatagagccaaaggactcaagcacgtggtccagtgcgtgttcgttgcaatccggaccattggaaacatcgtgatcgtgactactctgctgcagtttatgttcgcatgtattggagtgcagctcttcaagggaaagctgtatacctgctctgacagttccaagcagaccgaggcagaatgcaagggtaactacatcacttacaaggatggcgaagtggatcatccaatcatacagccacgcagttgggagaacagcaagtttgacttcgacaacgtgctggctgccatgatggctctcttcaccgtgtccacattcgaaggatggcctgaactcctctatagaagcatcgattctcatactgaggacaagggacccatctataactatcgggtcgagatttcaatcttcttcatcatatacatcatcatcatcgccttcttcatgatgaacatcttcgtgggcttcgtcatcgttacattccaggagcaaggcgagcaggagtacaagaattgcgaactcgacaagaaccagaggcagtgtgtggaatatgccctcaaagcaaggcctctcagacggtacatcccaaagaaccaacatcaatacaaagtctggtacgtggtcaactccacctactttgagtacctgatgtttgtcctgatactgctcaatacaatatgtctcgccatgcaacactatgggcaaagttgtctgttcaagatagcaatgaacatcctgaatatgctgtttactggcctgttcaccgtggaaatgatcctgaagctgatcgctttcaagcctaagcattacttctgcgacgcctggaatactttcgacgctctcatcgtcgttggatcaatcgtggacatagccatcacagaagtgaacccagccgagcacacacagtgcagcccatccatgaacgctgaagagaatagcaggataagcatcacattctttagactctttcgcgtgatgagactggtcaagctgctgtccagaggagagggcataaggaccctgctctggacattcataaagagcttccaagcactgccctatgttgctctgctgatcgtgatgctgttcttcatctacgcagtcatcggcatgcaagtgttcggcaagatcgcactgaatgacaccaccgagatcaatagaaacaacaacttccagacctttcctcaggctgtgctgctgctgtttcggtgtgctactggagaggcttggcaggacatcatgctcgcatgcatgccagggaagaaatgcgcaccagagagtgaacctagcaactctaccgaaggcgaaacaccctgcggttcaagtttcgctgtcttctacttcattagcttctacatgctctgcgcatttctgatcataaacctctttgttgctgtcatcatggacaatttcgactacctgactagagattggtccatcctgggtccacatcacctggacgagttcaaacgcatctgggctgagtacgacccagaggccaaaggacgcatcaagcatctggacgtggtgacactgctcagacggattcaaccaccactgggctttggaaagctctgccctcatcgggtcgcttgcaaacgcctggtgagcatgaatatgcctctcaatagtgatgggaccgttatgtttaacgcaacactgttcgctctggtgcgcacagccctcagaatcaagactgaaggaaacctggagcaagccaatgaagaactcagagccattatcaagaagatttggaagaggactagcatgaagctgctggatcaggttgttcctccagctggcgacgatgaagtcaccgtgggcaagttctatgcaacatttctcattcaggaatactttcggaaattcaagaagaggaaggagcaaggactcgtcgggaaacccagccagcgcaatgccctcagtctgcaagctgggctcagaacactgcatgacattggtcctgagatacgcagagccatctctggcgatctgacagctgaagaggagctggacaaagcaatgaaggaagccgtgtccgctgcctccgaggacgacatattccgcagagctggtggcctcttcgggaaccacgttagctattaccagtccgatggaagaagtgctttcccacagaccttcaccactcagaggccactgcatatcaataaggctggcagcagccaaggcgatacagagagtccatcccatgagaagctggtggatagtaccttcactcctagtagctacagtagcacaggcagcaacgccaatatcaacaatgccaacaacaccgctctcgggaggctcccaagaccagcaggataccctagcaccgtctccaccgtggagggacatggaccacctctgagcccagccattagggtgcaagaagtggcctggaagctgtcttctaaccgctgccatagtcgcgagtcccaggcagccatggctggccaggaagaaacatcacaggacgaaacttatgaagtgaagatgaaccacgataccgaggcttgttccgagccctcactcctgagcaccgagatgctgtcataccaggatgatgagaataggcagctgaccctcccagaggaagacaaacgggacatcagacagagcccaaagagaggtttcctgagaagcgcttcactgggcaggagagccagctttcatctcgaatgtctgaaacgccagaaagacagaggtggcgacattagccagaagacagtgctgcctctgcacctggtgcaccatcaggcactggcagtcgctgggctgagcccactgctgcagaggtcacatagtcctgcctctttccctagacctttcgccactccaccagccactcctggaagcagaggctggccaccacagccagttcccactctcagactcgaaggtgtggaatcatccgagaagctcaatagctcattcccaagcatccattgtggctcttgggcagagacaactcctggaggtggtggaagctctgctgctaggagagttaggccagtttcactgatggtgccctcccaggctggtgcaccaggacggcagtttcacggatctgcatcctctctggtggaggcagtcctcattagcgaagggctgggacagtttgcccaggacccaaagttcattgaagtgacaacccaagagctggcagacgcctgtgacatgacaatagaagaaatggaatcagctgctgacaacattctgagtggaggagcaccacaaagtccaaatggagcactgctgcccttcgtcaactgtagggatgctggtcaggatagagcaggtggcgaggaggatgcaggatgtgttagggctagaggtagaccatctgaagaggaactgcaggactccagagtgtacgtgagcagcctgtaagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 15 - DNA, 8659 bp, Artificial Sequence
  Synthetic
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagttaagaattctaagcggccgcatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagagctagccaccatgtatgatgattcctatgtgccaggtttcgaagactctgaggctggttctgcagatagctacacctctaggccaagcctggattccgacgtctccctggaggaggatcgcgaatccgctaggagagaagtcgagtctcaggctcaacagcagctggagagggctaagcataagcctgtggctttcgcagtgagaactaacgtgtcctactgcggagttctcgacgaagaatgtcctgttcagggatctggagtgaacttcgaggccaaggatttcctgcacatcaaggagaagtactctaacgattggtggatcggcaggctggtgaaagagggaggtgatatcgccttcatcccttctccacagaggctcgaatccattcgcctgaaacaggagcagaaagctagacgcagcggaaacccaagctctctgtccgacatcggcaataggagaagccctccaccttctctggctaagcagaagcagaaacaggctgaacacgtgcctccatacgacgttgtcccatccatgaggccagtggtgctggtgggaccttccctcaaaggctatgaagttaccgacatgatgcagaaggcactcttcgatttcctcaagcatagatttgacggtcgcatctctatcactagagtgactgccgatctctctctcgccaagcgctctgtcctgaacaatcctggaaagcgcactatcatcgaacgcagcagcgctaggtcctccattgccgaggtgcaatctgaaatcgagcgcatctttgagctggccaagagcctgcaactggtggtgctggacgcagacactatcaaccacccagctcaactcgcaaagacctccctcgcacccatcattgtgtttgttaaggtgagcagcccaaaggttctgcagaggctgattaggagcagaggcaaatctcagatgaagcacctcacagtgcagatgatggcctacgataagctggttcagtgcccacccgagtctttcgatgtgattctcgatgagaaccagctggaggatgcttgcgagcacctcgcagagtacctcgaagtctactggagagccactcatcatccagcaccaggacctggactgctcggaccaccttccgctatcccaggactgcagaaccagcagctcctgggtgaaagaggcgaggaacatagcccactcgaaagagactctctcatgccctctgatgaggcttccgagtccagcagacaggcatggaccggttcttcccagcgctccagcaggcacctcgaagaggattacgcagacgcataccaggacctgtaccagccacacagacagcacactagcggtctgcccagcgctaatggtcacgatccacaagatagactgctggctcaggactccgagcacaaccatagcgaccgcaactggcaacgcaatagaccttggccaaaggattcctacggatccggagccactaacttctctctgctcaagcaagcaggagacgtggaagaaaaccctggtcctatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 16 - DNA, 13630 bp, Artificial Sequence
  Synthetic
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggctgctggatgtctcctcgctctgactctcactctctttcaaagcctgctgattggaccatcctccgaggaacctttcccttctgctgtgaccatcaagtcctgggtcgataagatgcaggaggacctggtgaccctggctaagactgccagcggagtgaaccaactcgtcgatatctacgagaagtaccaagacctctacaccgtcgagccaaacaatgctcgccaactggtggagattgctgccagggatatcgagaagctcctcagcaatcgctctaaagcactggtcaggctcgctctcgaagctgagaaggtgcaggcagcccaccagtggagagaggacttcgcatctaacgaagtggtttactacaacgccaaagacgacctggacccagagaagaatgattctgaacctggctcccagaggatcaaacctgtcttcattgaagacgctaactttggacgccagattagctaccagcacgctgcagtccatatcccaactgacatctatgagggcagcactatcgttctcaatgaactgaattggacctctgcactggacgaagtgttcaagaagaatagggaggaggacccttctctgctgtggcaggtgttcggctccgcaacaggactcgctcgctactaccctgcttctccttgggtggataacagcaggactcccaacaagatcgacctgtacgatgtgagacgcagaccttggtacattcagggagcagccagccctaaagacatgctcatcctggtcgatgtttctggctccgtttctggtctgacactgaagctcatcagaacatctgtgtccgagatgctggagactctgtccgacgatgactttgtcaacgttgccagcttcaactccaacgcacaagatgtgtcctgctttcagcacctggtgcaagctaacgtcaggaacaagaaagtcctgaaggatgccgtgaacaacattaccgctaagggcattacagactacaagaaaggcttctctttcgccttcgagcagctcctgaactacaacgttagcagggcaaactgcaacaagatcatcatgctgtttacagatggtggcgaagagagagcacaggaaatcttcaacaagtacaacaaggacaagaaggtgagggtcttcacattctctgttggccagcataactacgacagaggtccaatccagtggatggcttgcgagaacaagggttactactacgagattccctctatcggtgcaattcgcatcaacacacaggaatacctggatgtcctgggtagacctatggtgctggctggtgacaaagctaagcaagtccagtggactaacgtgtatctcgacgctctcgagctcggactcgtgatcacaggtacactccctgtgttcaacattactggccagtttgagaacaagacaaacctcaagaatcaactcatcctgggtgtcatgggtgtggacgtcagcctggaggatatcaagagactcacacctcgctttacactgtgccctaatggctactactttgccatcgacccaaacggatacgtcctgctccatccaaacctccagcccaagaatcccaagtctcaagaacctgtgaccctcgactttctggacgcagagctggagaacgacatcaaagtggagatcagaaacaagatgatcgacggtgaatctggagagaagacattccgcaccctggtgaaatcccaggatgaacgctacatcgacaagggcaacaggacctacacttggactccagtcaacggcactgactattctctggcactcgtgctgcccacatactctttctactacatcaaggctaagctggaggaaaccattacccaggctaggagcaagaaaggcaagatgaaggactctgaaaccctgaagcctgacaactttgaggaatccggttacaccttcatcgctcccagagactattgcaacgacctcaagatttccgacaacaacacagagttcctgctcaacttcaatgagttcattgataggaagactcccaacaatccctcctgtaatgctgatctcatcaacagagtgctgctcgacgctggctttaccaacgagctggtccagaactattggtccaagcagaagaacatcaaaggcgtcaaggccagattcgtggtgactgatggaggcattactagggtttacccaaaggaagctggagagaattggcaagagaaccctgagacatacgaggatagcttctacaagagatccctggacaatgataactacgtgtttacagcaccttacttcaacaaatctggtccaggtgcttacgaaagcggaatcatggtgtccaaagccgtcgaaatctacatccaaggaaagctcctcaagccagcagtcgtgggcatcaagatcgacgtgaatagctggatcgagaacttcacaaagacatccattcgcgacccttgcgctggtcccgtgtgcgactgtaagcgcaactctgacgttatggactgtgttatcctcgacgacggaggattcctcctcatggctaatcatgacgactacactaatcagattggaagattcttcggtgagattgatccttccctcatgaggcacctggtcaacatctccgtgtacgctttcaacaagtcttacgactatcagtctgtgtgtgaacctggagcagctcctaaacagggtgctggtcaccgctctgcttatgtcccttccgttgccgacatcctgcagatcggctggtgggcaactgctgctgcttggtctattctgcagcagttcctgctgtctctgacatttcctcgcctcctggaagccgtcgagatggaagacgacgactttactgcaagcctgagcaagcagagctgtatcaccgaacagacccagtacttcttcgataacgactccaagtccttctctggtgtgctcgattgtggcaattgtagcagaatcttccacggtgagaaactgatgaacacaaatctgatcttcatcatggtggaatccaagggcacctgcccttgcgacacaagactgctgatccaagccgagcagacttctgatggacctaacccttgcgacatggtcaagcagccacgctataggaaaggacccgacgtctgctttgataacaacgtgctggaggattacactgactgtggaggcgtgtccggactcaacccaagcctgtggtacatcattggtatccagttcctgctcctctggctggtttccggttccacccacaggctgctgtaagcggccgcatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagagctagccaccatgtatgatgattcctatgtgccaggtttcgaagactctgaggctggttctgcagatagctacacctctaggccaagcctggattccgacgtctccctggaggaggatcgcgaatccgctaggagagaagtcgagtctcaggctcaacagcagctggagagggctaagcataagcctgtggctttcgcagtgagaactaacgtgtcctactgcggagttctcgacgaagaatgtcctgttcagggatctggagtgaacttcgaggccaaggatttcctgcacatcaaggagaagtactctaacgattggtggatcggcaggctggtgaaagagggaggtgatatcgccttcatcccttctccacagaggctcgaatccattcgcctgaaacaggagcagaaagctagacgcagcggaaacccaagctctctgtccgacatcggcaataggagaagccctccaccttctctggctaagcagaagcagaaacaggctgaacacgtgcctccatacgacgttgtcccatccatgaggccagtggtgctggtgggaccttccctcaaaggctatgaagttaccgacatgatgcagaaggcactcttcgatttcctcaagcatagatttgacggtcgcatctctatcactagagtgactgccgatctctctctcgccaagcgctctgtcctgaacaatcctggaaagcgcactatcatcgaacgcagcagcgctaggtcctccattgccgaggtgcaatctgaaatcgagcgcatctttgagctggccaagagcctgcaactggtggtgctggacgcagacactatcaaccacccagctcaactcgcaaagacctccctcgcacccatcattgtgtttgttaaggtgagcagcccaaaggttctgcagaggctgattaggagcagaggcaaatctcagatgaagcacctcacagtgcagatgatggcctacgataagctggttcagtgcccacccgagtctttcgatgtgattctcgatgagaaccagctggaggatgcttgcgagcacctcgcagagtacctcgaagtctactggagagccactcatcatccagcaccaggacctggactgctcggaccaccttccgctatcccaggactgcagaaccagcagctcctgggtgaaagaggcgaggaacatagcccactcgaaagagactctctcatgccctctgatgaggcttccgagtccagcagacaggcatggaccggttcttcccagcgctccagcaggcacctcgaagaggattacgcagacgcataccaggacctgtaccagccacacagacagcacactagcggtctgcccagcgctaatggtcacgatccacaagatagactgctggctcaggactccgagcacaaccatagcgaccgcaactggcaacgcaatagaccttggccaaaggattcctacggatccggagccactaacttctctctgctcaagcaagcaggagacgtggaagaaaaccctggtcctatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 17 - DNA, 13993 bp, Artificial Sequence
  Synthetic
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggctgctggatgtctcctcgctctgactctcactctctttcaaagcctgctgattggaccatcctccgaggaacctttcccttctgctgtgaccatcaagtcctgggtcgataagatgcaggaggacctggtgaccctggctaagactgccagcggagtgaaccaactcgtcgatatctacgagaagtaccaagacctctacaccgtcgagccaaacaatgctcgccaactggtggagattgctgccagggatatcgagaagctcctcagcaatcgctctaaagcactggtcaggctcgctctcgaagctgagaaggtgcaggcagcccaccagtggagagaggacttcgcatctaacgaagtggtttactacaacgccaaagacgacctggacccagagaagaatgattctgaacctggctcccagaggatcaaacctgtcttcattgaagacgctaactttggacgccagattagctaccagcacgctgcagtccatatcccaactgacatctatgagggcagcactatcgttctcaatgaactgaattggacctctgcactggacgaagtgttcaagaagaatagggaggaggacccttctctgctgtggcaggtgttcggctccgcaacaggactcgctcgctactaccctgcttctccttgggtggataacagcaggactcccaacaagatcgacctgtacgatgtgagacgcagaccttggtacattcagggagcagccagccctaaagacatgctcatcctggtcgatgtttctggctccgtttctggtctgacactgaagctcatcagaacatctgtgtccgagatgctggagactctgtccgacgatgactttgtcaacgttgccagcttcaactccaacgcacaagatgtgtcctgctttcagcacctggtgcaagctaacgtcaggaacaagaaagtcctgaaggatgccgtgaacaacattaccgctaagggcattacagactacaagaaaggcttctctttcgccttcgagcagctcctgaactacaacgttagcagggcaaactgcaacaagatcatcatgctgtttacagatggtggcgaagagagagcacaggaaatcttcaacaagtacaacaaggacaagaaggtgagggtcttcacattctctgttggccagcataactacgacagaggtccaatccagtggatggcttgcgagaacaagggttactactacgagattccctctatcggtgcaattcgcatcaacacacaggaatacctggatgtcctgggtagacctatggtgctggctggtgacaaagctaagcaagtccagtggactaacgtgtatctcgacgctctcgagctcggactcgtgatcacaggtacactccctgtgttcaacattactggccagtttgagaacaagacaaacctcaagaatcaactcatcctgggtgtcatgggtgtggacgtcagcctggaggatatcaagagactcacacctcgctttacactgtgccctaatggctactactttgccatcgacccaaacggatacgtcctgctccatccaaacctccagcccaagaatcccaagtctcaagaacctgtgaccctcgactttctggacgcagagctggagaacgacatcaaagtggagatcagaaacaagatgatcgacggtgaatctggagagaagacattccgcaccctggtgaaatcccaggatgaacgctacatcgacaagggcaacaggacctacacttggactccagtcaacggcactgactattctctggcactcgtgctgcccacatactctttctactacatcaaggctaagctggaggaaaccattacccaggctaggagcaagaaaggcaagatgaaggactctgaaaccctgaagcctgacaactttgaggaatccggttacaccttcatcgctcccagagactattgcaacgacctcaagatttccgacaacaacacagagttcctgctcaacttcaatgagttcattgataggaagactcccaacaatccctcctgtaatgctgatctcatcaacagagtgctgctcgacgctggctttaccaacgagctggtccagaactattggtccaagcagaagaacatcaaaggcgtcaaggccagattcgtggtgactgatggaggcattactagggtttacccaaaggaagctggagagaattggcaagagaaccctgagacatacgaggatagcttctacaagagatccctggacaatgataactacgtgtttacagcaccttacttcaacaaatctggtccaggtgcttacgaaagcggaatcatggtgtccaaagccgtcgaaatctacatccaaggaaagctcctcaagccagcagtcgtgggcatcaagatcgacgtgaatagctggatcgagaacttcacaaagacatccattcgcgacccttgcgctggtcccgtgtgcgactgtaagcgcaactctgacgttatggactgtgttatcctcgacgacggaggattcctcctcatggctaatcatgacgactacactaatcagattggaagattcttcggtgagattgatccttccctcatgaggcacctggtcaacatctccgtgtacgctttcaacaagtcttacgactatcagtctgtgtgtgaacctggagcagctcctaaacagggtgctggtcaccgctctgcttatgtcccttccgttgccgacatcctgcagatcggctggtgggcaactgctgctgcttggtctattctgcagcagttcctgctgtctctgacatttcctcgcctcctggaagccgtcgagatggaagacgacgactttactgcaagcctgagcaagcagagctgtatcaccgaacagacccagtacttcttcgataacgactccaagtccttctctggtgtgctcgattgtggcaattgtagcagaatcttccacggtgagaaactgatgaacacaaatctgatcttcatcatggtggaatccaagggcacctgcccttgcgacacaagactgctgatccaagccgagcagacttctgatggacctaacccttgcgacatggtcaagcagccacgctataggaaaggacccgacgtctgctttgataacaacgtgctggaggattacactgactgtggaggcgtgtccggactcaacccaagcctgtggtacatcattggtatccagttcctgctcctctggctggtttccggttccacccacaggctgctgtaagcggccgcatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagagctagccaccatgcaatgttgtggactggttcatagacgcagagttagagtgtcttacggctccgctgactcctacacaagcaggccatctgacagcgatgtctccctggaagaagaccgggaggcagtgagacgcgaggctgagcggcaggctcaggcccagctggaaaaggcaaagaccaaaccagtggcttttgccgtgcggactaacgtgagctatagcgcagctcacgaagatgacgtcccagtccccggcatggcaattagcttcgaggcaaaggactttctgcatgtgaaggagaagttcaacaatgactggtggatcgggcgcctggtcaaggaaggctgtgagatcggctttattccttccccagtcaaactcgaaaacatgagactgcaacacgagcaaagggccaaacaaggaaagttttattccagcaagagcggcggcaacagctccagcagcctcggagatatcgtgcctagcagcaggaagtccacacctcccagcagcgcaattgacatcgatgctactgggctggatgcagaagagaacgacatcccagcaaatcaccggtcccctaaaccctctgccaacagcgtgacctctccccatagcaaagagaagcggatgcctttcttcaagaagaccgagcacacccctccctacgacgtcgtcccttctatgcggccagttgttctggttggaccaagcctgaaagggtacgaagtgaccgatatgatgcaaaaagccctgtttgactttctcaaacaccgctttgagggcagaatctccattacacgggtcaccgcagatattagcctcgccaagagatccgtcctgaataatccttccaaacacgccatcatagagaggtccaacaccaggtcttccctggccgaggtgcagtccgagattgagcgcattttcgaactcgctaggaccctgcaactggttgtgctggatgccgataccattaatcaccccgcccagctgtctaaaactagcctggctcctattatagtgtacgttaagatatcatcaccaaaggttctgcagagactgatcaaatctaggggcaagagccaagctaagcacctcaacgtgcagatggtggccgcagataaactcgcacagtgccccccagaactgttcgacgtcatcctggatgagaaccagctcgaagacgcttgtgaacatctcgcagactacctggaggcatattggaaggctacccaccccccttccagctcactgcctaatccactgctgtcaaggacactggctacatcatccctgccactcagccccaccctcgcaagcaattcccagggatctcagggggaccagaggactgatcgctccgcccccatcaggtcagcttcccaggccgaagaagaacctagcgtggagccagtgaagaaaagccagcatagatcatcttctagcgccccccaccataaccatcgctcaggcacatcccggggactctctcgccaggagacttttgattccgagacacaggaatccagggattcagcctatgtggagcccaaggaggattattcccatgaccatgtggatcactatgcctctcaccgggaccacaatcaccgcgacgaaactcacggttcttctgatcacagacaccgggagagccggcatcggtctagggacgtcgatcgcgaacaagaccataatgagtgcaacaagcaacggagccgccataaaagcaaggacagatactgtgagaaagacggcgaagtcatcagcaagaaaagaaacgaagccggggagtggaacagagacgtctacatccggcaaggatccggagccactaacttctctctgctcaagcaagcaggagacgtggaagaaaaccctggtcctatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

(19) United States
(12) Patent Application Publication
(10) Pub. No.: US 2024/0101632 A1
(43) Pub. Date: Mar. 28, 2024

(54) GENETICALLY ENGINEERED ELECTRICALLY-STIMULATED EFFECTOR CELLS FOR IN SITU SYNTHESIS OF PROTEINS

(71) Applicant: SRI International, Menlo Park, CA (US)

(72) Inventors: Parijat BHATNAGAR, Menlo Park, CA (US); Sherri NEWMYER, Menlo Park, CA (US)

(73) Assignee: SRI International, Menlo Park, CA (US)

(21) Appl. No.: 18/274,255

(22) PCT Filed: Jan. 27, 2022
(86) PCT No.: PCT/US2022/014161

§ 371 Date: Jul. 26, 2023

Related U.S. Application Data
(60) Provisional application No. 63/142,311, filed on Jan. 27, 2021.

(51) Int. Cl. C07K 14/555; A61K 35/17; A61K 41/00; A61P 31/14

(52) U.S. Cl. CPC C07K 14/555; A61K 35/17; A61K 41/00; A61P 31/14; A61K 38/00

(57) ABSTRACT

An example genetically engineered electrically-stimulated (ES) cell comprises an exogenous polynucleotide sequence that includes an electrical-sensor element, an actuator element, and an effector element. The electrical-sensor element encodes a voltage-gated calcium ion channel (CaV), wherein the CaV is configured to transition from a closed state to an open state in response to stimulation. The actuator element encodes a transcription factor binding site that upregulates synthesis of an effector protein. The effector element encodes the effector protein, wherein, in response to the transition of the CaV to the open state, the genetically engineered ES effector cell is configured to activate and, to synthesize and secrete the effector protein.

### GOVERNMENT RIGHTS

This invention was made with Government support under contract no. D19AP00024 awarded by the Defense Advanced Research Projects Agency. The Government has certain rights in this invention.

### INCORPORATION-BY-REFERENCE OF MATERIAL SUBMITTED ELECTRONICALLY

Incorporated by reference in its entirety is a computer-readable nucleotide sequence listing, an ASCII text file which is 144 kb in size, submitted concurrently herewith, and identified as follows: “S1647133111_SequenceListing_ST25” and created on Jan. 25, 2022.

### BACKGROUND

Various standard-of-care therapeutics are designed to treat a disease at the time of diagnosis. Small molecule and biologics are the current standard of care for many immunological diseases (e.g., viral diseases, cancers, autoimmune disorders, etc.). For example, for viral diseases, currently vaccination is the most effective means of treating emerging and cycling viruses that cause disease and, even, death. Unfortunately, vaccine development is resource-intensive and time-consuming, requiring identification of the virus-specific antigen and determination of safe and effective administration for each newly emerging pathogen. Further, variants evolve that may escape vaccine-induced immunity to trigger new waves of illness and undo the vaccine development efforts.

### SUMMARY

The present invention is directed to overcoming the above-mentioned challenges and others related to therapeutics and other purposes, such as involving a genetically engineered electrically-stimulated (ES) effector cell line which can electrically activated in situ to cause synthesis of an effector protein at a target location. Examples are not limited to therapeutics, and may be include research, surgery or other detection implementations, among other implementations.

Various aspects are directed to a genetically engineered electrically-stimulated (ES) effector cell comprising an exogenous polynucleotide sequence that includes, in operative association: an electrical-sensor element that encodes a voltage-gated calcium ion channel (CaV), wherein the CaV is configured to transition from a closed state to an open state in response to electrical stimulation; an actuator element that encodes a transcription factor binding site that upregulates synthesis of an effector protein in response to the transition of the CaV to the open state; and an effector element that encodes the effector protein, wherein, in response to the transition of the CaV to the open state, the genetically engineered ES effector cell is configured to activate and, to synthesize and secrete the effector protein.

In some aspects, the genetically engineered ES effector cell is configured to activate by causing an influx of Ca⁺² in response to the electrical stimulation and the transition of the CaV to the open state, and the influx of Ca⁺² activates the transcription factor binding site and causes the upregulation of the synthesis of the effector protein.

In some aspects, the genetically engineered ES effector cell comprises a T-cell, a natural killer (NK) cell, a pluripotent stem cell, a multipotent stem cell, an epithelial cell, or a K562 cell.

In some aspects, the effector element further encodes a signal peptide upstream from the effector protein that is non-native to the effector protein.

In some aspects, the effector protein is selected from the group consisting of: a therapeutic protein, a detectable reporter peptide, a downstream signaling protein, and a combination thereof.

In some aspects, the exogenous polynucleotide sequence further encodes a detectable reporter peptide.

In some aspects, the detectable reporter peptide is selected from the group consisting of: luciferase or a bioluminescent variant thereof, Green Fluorescent Protein (GFP) or a fluorescent variant thereof, and lacZ or a colorimetric variant thereof.

In some aspects, the CaV includes a pore-forming subunit configured to form a channel in a cell surface of the genetically engineered ES effector cell and a set of auxiliary subunits configured to regulate the transition of a channel opening of the CaV and traffic the CaV to the cell surface.

In some aspects, the effector protein includes a therapeutic protein selected from the group consisting of: a Type-I interferon (IFN), a Type-III IFN, and a combination thereof.

In some aspects, CaV includes a pore-forming subunit selected from the group consisting of: CaV1.2, CaV1.3, and variants thereof.

In some aspects, the transcription factor binding site is selected from the group consisting of: a nuclear factor of activated T-cell (NFAT) response element, a serum response element (SRE), and a cyclic AMP response element (CRE).

In some aspects, the effector protein includes a therapeutic protein configured to act on a Severe Acute Respiratory Syndrome Coronavirus 2 (SARS-CoV-2)-infected cell.

Various aspects are directed to a population of genetically engineered ES effector cells, each of the genetically engineered ES effector cells of the population comprising an exogenous polynucleotide sequence that includes an actuator element, an effector element, and an electrical-sensor element, wherein: the electrical-sensor element encodes a CaV including a pore forming sub-unit and a set of auxiliary subunits, wherein the CaV is configured to transition from a closed state to an open state in response to electrical stimulation and the set of auxiliary subunits are configured to regulate the of the CaV channel and trafficking of the CaV to the surface of the genetically engineered ES effector cell; the actuator element encodes a transcription factor binding site that upregulates synthesis of an effector protein in response to the transition of the CaV to the open state; and the effector element encodes the effector protein operably, wherein, in response to the transition of the CaV to the open state, the genetically engineered ES effector cell is configured to activate and, to synthesize and secrete the effector protein.

In some aspects, the set of auxiliary subunits includes a α₂δ subunit and a β subunit.

In some aspects, the set of auxiliary subunits includes a α₂δ₁ subunit and a β₃ subunit.

In some aspects, wherein the population of genetically engineered ES effector cells are configured to provide a calibrated amount of the effector protein as a function of the electrical stimulation applied and a duration of the electrical stimulation applied.

Various aspects are directed to a method comprising contacting a plurality of cells with a volume of a genetically engineered ES effector cell, wherein the genetically engineered ES effector cell comprises a polynucleotide sequence that includes: an electrical-sensor element that encodes a CaV including a pore-forming subunit and a set of auxiliary subunits; an actuator element that encodes a transcription factor binding site; and an effector element that encodes an effector protein; The method further includes after contacting the plurality of cells with the volume of the genetically engineered ES effector cell, applying an electric field to the volume of the genetically engineered ES effector cell to electrically stimulate the CaV; and in response to the electrical stimulation, causing the CaV to transition from a closed state to an open state, initiating expression of the effector protein by the actuator element, and secreting the effector protein by a signal peptide.

In some aspects, the method further includes locating electrical circuitry including two electrodes coupled to a power supply proximal to the volume of the genetically engineered effector ES cell and applying the electric field by applying a voltage between the two electrodes.

In some aspects, the amount of secreted effector protein is provided as a function of the electric field applied to the genetically engineered ES effector cell by the electrical stimulation and a duration of the electrical stimulation applied.

In some aspects, the method further includes activating the genetically modified ES effector cell as a function of at least one of: a voltage, a total duration, a pulse duration, and a frequency of the electrical stimulation.

### BRIEF DESCRIPTION OF THE DRAWINGS

Various example embodiments can be more completely understood in consideration of the following detailed description in connection with the accompanying drawings, in which:

FIG. 1 illustrates an example of a genetically engineered ES effector cell, in accordance with the present disclosure.

FIG. 2 illustrates an example of a genetically engineered ES effector cell and a sequence of events triggered in situ when, in accordance with the present disclosure.

FIG. 3 illustrates an example of a population of genetically engineered ES effector cells, in accordance with the present disclosure.

FIG. 4 illustrates an example method of contacting a plurality of cells with a volume of a genetically engineered ES effector cell, in accordance with the present disclosure.

FIGS. 5A-5D illustrate example plots characterizing activation of genetically engineered ES effector cells by electrical stimulation, in accordance with the present disclosure.

FIGS. 6A-6B illustrate example plots characterizing activation of a genetically engineered ES effector cell as compared to a reporter cell, in accordance with the present disclosure.

FIGS. 7A-7D illustrate example electrical parameters used to drive expression of the effector protein from a genetically engineered EA effector cell, in accordance with the present disclosure.

FIGS. 8A-8D illustrate example biological parameters used to drive expression of the effector protein from a genetically engineered ES effector cell, in accordance with the present disclosure.

FIGS. 9A-9C illustrate example results of genetically engineered ES effector cells with different pore-forming subunits and auxiliary subunits, in accordance with the present disclosure.

FIGS. 10A-10B illustrate example results of using different electrode distances for electrically stimulating a genetically engineered ES effector cell, in accordance with the present disclosure.

FIG. 11 illustrates example cell viability as a function of voltage application for electrically stimulating a genetically engineered ES effector cell, in accordance with the present disclosure.

FIGS. 12A-12B illustrate example results of using different durations for electrically stimulating a genetically engineered ES effector cell, in accordance with the present disclosure.

FIG. 13 illustrates example results of using different frequencies for electrically stimulating genetically engineered ES effector cells, in accordance with the present disclosure.

FIGS. 14A-14B illustrate example results of using different durations for electrically stimulating a genetically engineered ES effector cell, in accordance with the present disclosure.

FIGS. 15A-15B illustrate example plasmids for generating genetically engineered ES effector cells, in accordance with the present disclosure.

FIGS. 16A-16B illustrate example results of electrically stimulating genetically engineered ES effector cells, in accordance with the present disclosure.

FIGS. 17A-17B illustrate example results of electrically stimulating genetically engineered ES effector cells, in accordance with the present disclosure.

FIGS. 18A-18B illustrate example results of electrically stimulating genetically engineered ES effector cells, in accordance with the present disclosure.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

In some embodiments, a cell can be engineered to express genetic elements including an actuator element, an effector element, and a voltage-gated calcium ion channel (CaV) that regulates the intracellular transcriptional machinery of the cell via electrical stimulation, herein referred to as “a genetically engineered ES effector cell”. The CaV is electrically sensitive, and in response to electrical stimulation (e.g., a particular electrical pulse input), the cell activates a downstream pathway leading up to synthesis of one or more particular proteins via the actuator element and the effector element. The genetic elements of the ES effector cell can be modular and/or a cell can include multiple genetic elements to yield a genetically engineered ES effector cell having the capacity to serve as a vector for a variety of in vitro, ex vivo, and in vivo applications. Such cells can be modular in that parts can be conserved, and parts can be changed for different applications. Multiple types of genetically engineered ES effector cells can provide a robust, reproducible cellular system to therapeutically target complex diseases in vivo. Such genetically engineered ES effector cells also provide a reliable in vivo imaging technology and a reliable, in vitro sensor technology in a variety of applications.

Embodiments in accordance with the present disclosure are directed to a genetically engineered ES effector cell which is used as a cellular chassis or vector to act as a biofactory for different target proteins. A biofactory, as used herein, includes and/or refers to a living cell which is genetically engineered to produce one or more proteins, and can be interchangeably referred to as a genetically engineered ES effector cell. The genetically engineered ES effector cell respond to electrical stimulation and, in response, to activate expression of a particular protein or multiple proteins. The genetically engineered ES effector cell can be used as a therapeutic to treat severe cases of disease and can also be used as a prophylactic to prevent the progression of mild/moderate to severe cases of disease, including cancers, emerging pathogens, and others that evade the immune system or involve its malfunction. In some embodiments, the biofactory can result in readily available stockpile of broad-spectrum antiviral cells that, in event of an epidemic of bioweapon attack, can be rapidly deployed and activated using extracorporeal devices without the need for developing vaccines specific for each viral pathogen.

As used herein, a “genetically engineered ES effector cell” and/or “ES effector cell” includes and/or refers to a cell that is genetically engineered or modified to comprise (i) an electrical-sensor element, (ii) an actuator element, and (iii) an effector element, each of which can be modular. As used herein, the terms “modular” and “modularity” include and/or refer to the versatility associated with recombinant sequence domains and the resulting recombinant polypeptides when assembled in various combinations for introduction into a cell.

As used herein, “electrical-sensor element” includes and/or refers to a polynucleotide sequence encoding a CaV that is configured to transition from a closed state to an open state in response to electrical stimulation, which can be used to synthesize proteins. CaVs are heteromultimeric protein complexes that contain a pore-forming subunit that defines the channel type and a set of auxiliary subunits that alter the function of the pore-forming subunit and also regulate trafficking of the channel to the plasma membrane of the ES effector cell. In various embodiments, the CaV includes a pore-forming subunit (e.g., α1) configured to form a channel in the effector cell surface and a set of auxiliary subunits (e.g., a α₂δ and β subunits) configured to regulate the transition of a channel opening of the CaV and traffic the CaV to a surface of the genetically engineered ES effector cell. CaVs are normally in the closed state, in which the pore-like channel of the CaV is closed and, in response to electrical stimulation, transition to the open state, in which the pore-like channel is opened and which allows for an influx of Ca²⁺ into the genetically engineered ES effector cell. The influx of Ca²⁺ triggers the synthesis of engineered proteins in situ, herein sometimes referred to as “effector proteins” via the activation of the actuator element that encodes a transcription factor binding site that initiates transcription and translation events, as further described herein.

As used herein, “actuator element” includes and/or refers to a polynucleotide sequence encoding a transcription factor binding site that initiates transcription and translation events downstream of a triggering signal (e.g., transition of the CaV in response to electrical stimulation). In general, the underlying molecular mechanism of the actuator element is based on the intracellular calcium [Ca²⁺]ᵢ dynamics, a mechanism used by almost all types of cells to regulate their functions. Exemplary response elements include, without limitation, nuclear factor of activated T-cells (NFAT) response element (NFAT-RE), serum response element (SRE), and cyclic AMP response element (CRE).

As used herein, “effector element” includes and/or refers to a polynucleotide sequence encoding an effector protein, and in some instances, an effector protein operably linked to a signal peptide. The polynucleotide sequence encoding the effector protein can be, for example, a sequence derived from a human gene, a sequence derived from a gene of a non-human species, a recombinant sequence, a sequence encoding a detectable reporter molecule, a sequence encoding a detectable imaging molecule, a sequence encoding a therapeutic molecule, and the like.

The genetically engineered ES effector cell into which the electrical-sensor element, the actuator element, and the effector element are introduced can be any cell type including human cells or non-human cells (e.g., mammal, reptiles, plants, among others). In this manner, the genetically modified cellular “source” of the modular elements provides a cellular chassis or frame providing, among other things, transcriptional and translational machinery for expression and presentation of the electrical-sensor element, the actuator element, and the effector element. In some embodiments, the ES effector cells can be from a source (e.g., a first human), modified, and administered to an organism that is different than the source (e.g., the host which is a second human). In other embodiments, the effectors cells can be from the source (e.g., a first human), modified, and administered back to the source (e.g., the source is the host).

A variety of different types of cells can be modified to form the genetically engineered ES effector cell. For example, the effector cell can include a T-cell. Other cell types that can be used include without limitation other immune cells (e.g., primary T-cells, natural killer cells), pluripotent stem cells (e.g., iPS cells), mesenchymal stem cells, hematopoietic stem cells, epithelial cells, HEK293 cells, CHO cells, and the like.

In addition to modularity, the genetically engineered ES effector cells can be modified for a variety of application by providing multiple (e.g., two or more) of some of the modular elements or all of the modular elements. For example, a genetically engineered effector cell can be designed to comprise multiple actuator elements (e.g., two or more actuator elements), and/or effector elements (e.g., a detectable reporter polypeptide, a detectable imaging polypeptide, and a therapeutic polypeptide). In some cases, multiplicity takes the form of providing multiple ES effector cells (e.g., a plurality of cells) to a subject to provide more than one therapeutic “task” for treating or preventing a disease. The artificial cell-signaling pathway of such genetically engineered ES effector cells can introduce the capability to serve as vector by producing calibrated amounts of protein-based therapeutics and inducing intended autocrine and paracrine signaling, upon the genetically engineered ES effector cell being triggered by electrical stimulus. The genetically engineered ES effector cell can allow for focused synthesis of the biologics at the target location, target time, and/or extend treatment duration for better patient outcome by limiting systemic toxicity.

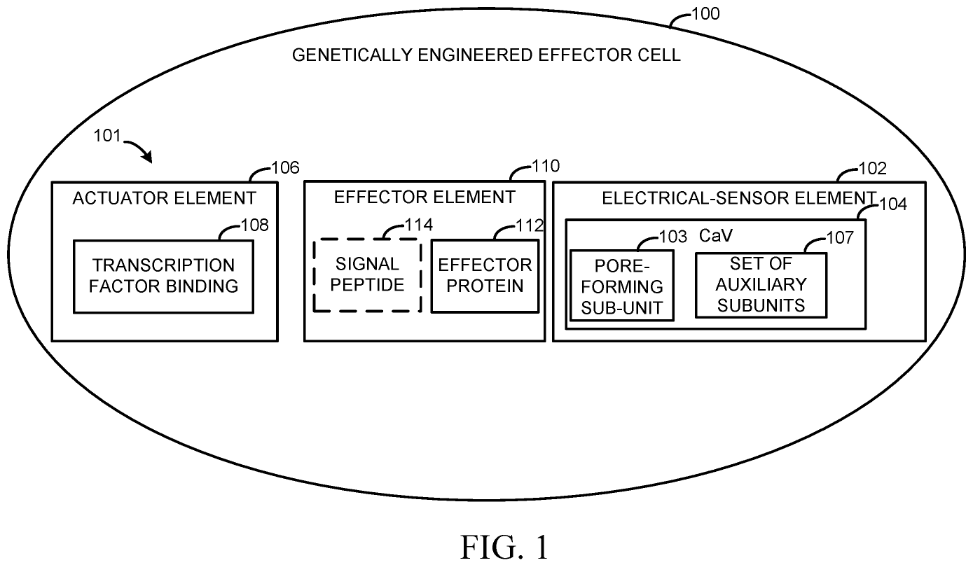

Turning now to the figures, FIG. 1 illustrates an example genetically engineered ES effector cell, in accordance with the present disclosure. The genetically engineered ES effector cell 100, herein generally referred to as “the ES effector cell” for ease of reference, can be modular in that genetic elements 102, 106, 110 can be adjusted for different purposes and to synthesize different effector proteins.

The ES effector cell 100 comprises an exogenous polynucleotide sequence 101 that includes, in operative association, an electrical-sensor element 102, an actuator element 106, and an effector element 110. A variety of different types of cells can be used to generate the ES effector cell 100, such as a T-cell, a natural killer cell, a pluripotent stem cell, a multipotent stem cell, an epithelial cell, or a K562 cell, among others. The cell modified to generate ES effector cell 100 can include a living cell from an organism, such as a basic membrane-bound unit that contains structural and functional elements. In some embodiments, the exogenous polynucleotide sequence 101 can be selected from SEQ ID NOs: 12-17. However, embodiments are not so limited and the exogenous polynucleotide sequence 101 can include other sequences, such as a sequence with at least 80 percent (%), 85%, 90%, 95%, or 99% sequence identity to one of the sequences set forth in SEQ ID NOs: 12-17, among other sequences.

The electrical-sensor element 102 encodes a CaV 104. As used herein, a CaV includes and/or refers to an artificially constructed heteromultimeric protein complex which forms a channel in the cell surface of the ES effector cell 100. The CaV 104 is configured to transition from a closed state to an open state in response to electrical stimulation. For example, the CaV 104 includes a pore-forming subunit 103, sometimes referred to as α1, and a set of auxiliary subunits 107. The pore-forming subunit 103 is configured to form a channel in the effector cell surface. Example pore forming sub-units include CaV1.2 and CaV1.3. Other example pore forming sub-units include CaV 1.1, CaV2.1, CaV2.2, CaV2.3, CaV3.1, CaV3.2, and CaV3.3. The set of auxiliary subunits 107 are configured to regulate the transition to the open state (e.g., opening of the channel) of the CaV 104 and traffic the CaV 104 (e.g., the channel) to a surface of the ES effector cell 100. Example auxiliary subunits include α₂δ and R subunits. In some examples, the set of auxiliary subunits includes a α₂δ₁ subunit and a β₃ subunit. In some embodiments, the pore-forming subunit 103 can be encoded by and/or include SEQ ID NO: 1 or SEQ ID NO: 2. In some embodiments, the set of auxiliary subunits 107 can be encoded by and/or include SEQ ID NO: 3, SEQ ID NO: 4, SEQ ID NO: 5, or a combination thereof, such as SEQ ID NO: 3 and SEQ ID NO: 5, or SEQ ID NO: 4 and SEQ ID NO: 5. As such, in some embodiments, the electrical-sensor element 102 can include the combination of SEQ ID NO: 1 or SEQ ID NO: 2, SEQ ID NO: 5, and SEQ ID NO: 3 or SEQ ID NO: 4. However, embodiments are not so limited and the electrical-sensor element 102 can include other sequences, such as a sequence with at least 80%, 85%, 90%, 95%, or 99% sequence identity to one or more of the sequences set forth in SEQ ID NOs: 1-5, among other sequences.

In the closed state, the channel of the CaV 104 can be closed. In response to an electrical stimulation, the CaV 104 transitions to the open state in which the channel is open and Ca²⁺ ions can travel through the channel. The open state can mobilize internal Ca⁺² stores for intracellular Ca⁺² release. As further described below, the influx of Ca⁺² activates the ES effector cell 100 and causes synthesis of an effector protein 112. As the synthesis is in response to the electrical stimulation, the amount of the effector protein 112 synthesized can be controlled by the electrical stimulation, as further described herein.

The actuator element 106 encodes a transcription factor binding site 108. As further described herein, the ES effector cell 100 is configured to activate by causing an influx of Ca⁺² in response to the electrical stimulation and the transition of the CaV 104 to the open state, and the influx of Ca⁺² activates the transcription factor binding site 108 (e.g., a nuclear factor) and causes the upregulation of the synthesis of the effector protein 112. The transcription factor binding site 108 includes and/or refers to binding site for a protein that upregulates synthesis of an effector protein 112 in response to the transition of the CaV 104 to the open state. The transcription factor binding site 108 can bind to transcription factors as triggered by [Ca²⁺], which as described above, release in response to the electrical stimulation and the transition of the CaV 104. In some embodiments, the transcription factor binding site 108 is selected from a NFAT-RE, a serum response element (SRE), and a cyclic AMP response element (CRE). The actuator element 106 can thereby include a sequence for binding the factors triggered by [Ca²⁺], and can trigger amplified synthesis of the effector protein 112 in response to [Ca²⁺]ᵢ rise.

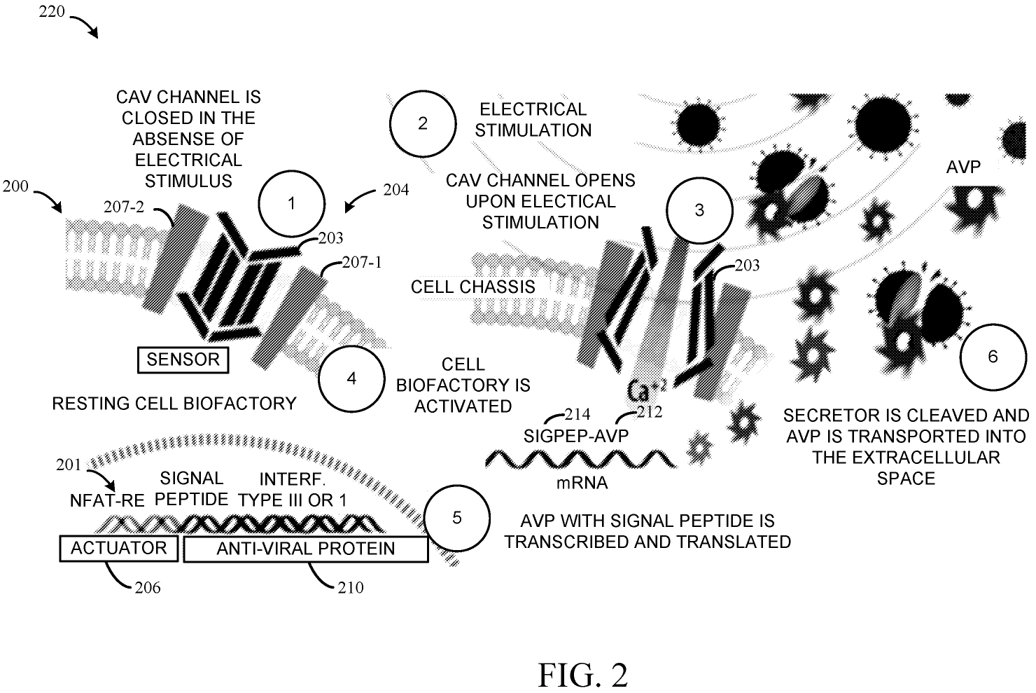

In some embodiments, the actuator element 106 encodes at least one NFAT transcription factor binding site for a transcription factor protein. In some example, the actuator element 106 encodes a plurality of transcription factor binding sites, such as at least two transcription factor binding sites, three transcription factor binding sites, or six transcription factor binding sites, among other amounts. The NFAT transcription factor family consists of five members NFATc1, NFATc2, NFATc3, NFATc4, and NFAT5. See Sharma S et al. (2011) PNAS, 108(28); Hogan P G et al. (2010) Ann Rev Immunol, 28; Rao A, Hogan P G (2009) Immunol Rev, 231(1); Rao A (2009) Nat Immunol, 10(1), M. R. Müller and A. Rao, Nature Reviews Immunology, 2010, 10, 645-656; M. Oh-Hora and A. Rao, Curr. Opin. Immunol., 2008, 20, 250-258. Crabtree & Olson E N (April 2002), Cell 109 Suppl (2): S67-79, which are each hereby incorporated herein in their entireties for their teaching. NFATc1 through NFATc4 are regulated by calcium signaling. Calcium signaling is critical to NFAT activation because calmodulin, a well-known calcium sensor protein, activates the serine/threonine phosphatase calcineurin. The underlying molecular mechanism of this strategy is based on intracellular Ca⁺² ([Ca²⁺]ᵢ) dynamics (as further shown by FIG. 2). The [Ca²⁺]ᵢ dynamics are common to almost all cell types, and the embodiments are thus broadly applicable. The [Ca²⁺]ᵢ rise from CAR-mediated stimulation of cells leads to dephosphorylation of the nuclear factor of an activated ES effector cell 100 (through Ca⁺²/calmodulin-dependent serine phosphatase calcineurin), which is then translocated to the nucleus and interacts with the NFAT-RE to upregulate expression of the effector protein 112. In parallel, the NFAT-RE performs its natural function of inducing Interleukin-2 in the activated ES effector cell 100.

The effector element 110 encodes the effector protein 112, and in some embodiments, encodes the effector protein 112 operably linked to a signal peptide 114. As further illustrated herein, in some embodiments, the signal peptide 114 is upstream of the effector protein 112. The signal peptide 114 can be non-native to the effector protein 112. For example, the effector protein 112 can be unable to secrete into the extracellular environment without the addition of the signal peptide 114. However, embodiments are not so limited and in some embodiments, the effector protein 112 includes a native signal peptide. For example, the effector protein 112 can (natively) include the signal peptide 114. In other examples, the native signal peptide of the effector protein 112 can be removed and a non-native signal peptide 114 can be added. In some embodiments, the effector protein 112 can be encoded by and/or include SEQ ID NO: 6, SEQ ID NO: 7, SEQ ID NO: 8 or SEQ ID NO: 10. As such, in some embodiments, the effector element 110 can include one or more of SEQ ID NO: 6, SEQ ID NO: 7, SEQ ID NO: 8, and SEQ ID NO: 10. However, embodiments are not so limited and the effector element 110 can include other sequences, such as a sequence with at least 80%, 85%, 90%, 95%, or 99% sequence identity to one of the sequences set forth in SEQ ID NOs: 6-10, among other sequences.

As used herein, the terms “secretor”, “secretory peptide and “signal peptide” are used interchangeable and include and/or refer to a peptide that assists or directs the synthesized effector protein 112 into the extracellular environment (e.g., assists with translocating the effector element 110). The signal peptide 114 can be operably linked or fused to the effector protein 112 for release into the extracellular environment. In this manner, the signal peptide 114 can direct movement of the effector protein 112 outside of the ES effector cell 100. A signal peptide 114 is particularly advantageous when included in the ES effector cell 100 expressing an effector protein 112 that is unable to and/or minimally able to translocate natively, where the effector protein 112 can remain inside the ES effector cell 100 in the absence of the signal peptide 114 and/or can translocate at a rate below a threshold. Generally, signal peptides are located at the N-terminus of nascent secreted proteins and characteristically have three domains: (1) a basic domain at the N-terminus, (2) a central hydrophobic core, and (3) a carboxy-terminal cleavage region. Any appropriate signal peptide can be used. For example, the signal peptide 114 can be the signal peptide of Interleukin-6 or Interleukin-2.

In various embodiments, in response to the transition of the CaV 104 to the open state, the engineered ES effector cell 100 is configured to activate, and to synthesize and secrete the effector protein 112. For example, the ES effector cell 100 can synthesize and secrete an amount of the effector protein 112 as a function of the electrical stimulation applied and a duration of the electrical stimulation applied, as further described herein.

The effector protein 112 can include a variety of different types of proteins. For example, the effector protein 112 can include a detectable reporter protein, a therapeutic protein, a downstream signaling protein, and a combination thereof. As used herein, a detectable reporter protein includes and/or refers to a protein that is detectable upon expression, such as a protein that provides an optical, electrical or other type of detectable signal. Example detectable reporter peptides include luciferase or a bioluminescent variant thereof, Green Fluorescent Protein (GFP) or a fluorescent variant thereof, and lacZ or a colorimetric variant thereof. A therapeutic protein includes and/or refers to a protein that provides a therapeutic effect to a subject, such as a host and/or patient. Example therapeutic proteins include an antiviral protein, an antitumor protein, a cytotoxic protein, an immunostimulatory protein, and an immunosuppressive protein. A downstream signaling protein includes and/or refers to a protein that drives downstream elements of a signaling pathway, such as for regulation of cell growth, proliferation, differentiation, and apoptosis.

Non-limiting examples of effector proteins include cytotoxic polypeptides of bacterial origin (e.g., parasporin, plantaricin A); insect origin (e.g. Polybia-MP1); antiviral polypeptides from viral origin (e.g., α-helical peptide (AHP)); antiviral polypeptides from viral origin (e.g., anti-viral peptides (AVP)); immunosuppressive peptides of fungal origin (e.g., colutellin A); vasodilators (e.g., relaxin, bradykinin) and endopeptidase (e.g., heparanase, relaxin, collagenase); and cell-penetrating cationic peptides (e.g., LL-37, TAT peptide).

As noted above, in some embodiments, the effector protein 112 is a therapeutic protein. The therapeutic protein can act directly on the target cell, in some embodiments. In other embodiments, the therapeutic protein can act on cells adjacent to the target cell or on non-cellular components. Example therapeutic proteins include a cytotoxic protein, an antitumor protein, an immunostimulatory protein, and an immunosuppressive protein, among others.

In some specific examples, the effector protein 112 includes a therapeutic protein comprising an antiviral protein. For example, the effector protein 112 can include a Type-I interferon (IFN), a Type-III IFN, and a combination thereof, and which can be used to treat a patient for a viral infection. For example, the effector protein 112 can include a therapeutic protein configured to kill or otherwise cause action on a Severe Acute Respiratory Syndrome Coronavirus 2 (SARS-CoV-2)-infected cell. However, examples are not so limited.

Different parts of the genetic elements 102, 106, 110 of the ES effector cell 100 can be modular and other parts can be conserved (e.g., may not change for different implementations). For example, in some embodiments, the electrical-sensor element 102, the actuator element 106, and the optional signal peptide 114 are constant domains, and the effector protein 112 is a variable domain. As an example, the effector protein 112 can be changed to cause in situ synthesis of different proteins, while the electrical-sensor element 102, the actuator element 106, and/or the signal peptide 114 remain the same for the different implementations. Keeping parts conserved can reduce production time. However, embodiments are not so limited, and any part of the ES effector cell 100 can be modified.

In some embodiments, the ES effector cell 100 can include multiple (e.g., two or more) of some or all of the genetic elements 102, 106, 110. For example, the ES effector cell 100 can include multiple actuator elements 106, and/or multiple effector elements 110. In some embodiments, multiplicity takes the form of providing multiple genetically engineered ES effector cells (e.g., a plurality of cells) modified as described herein to a host to provide more than one tasks for treating or preventing a disease and/or for other purposes, such as research.

In some embodiments, the exogenous polynucleotide sequence 101 encodes for more than one effector protein. For example, the exogenous polynucleotide sequence 101 can further encode a detectable reporter peptide. In some embodiments, the ES effector cell 100 can include an additional effector element encoding the detectable reporter peptide and/or the effector element 110 can further encode the detectable reporter peptide. For example, the detectable reporter peptide can be linked to the effector protein 112 by a 2A linker peptide. In some embodiments, the effector element 110 can include multiple therapeutic proteins, such as two therapeutic proteins linked by a 2A linker peptide. A 2A linker peptide, as used herein, includes and/or refers to a peptide which induces ribosomal skipping during translation of a protein complex (e.g., encoding of two proteins or peptides linked by the 2A linker peptide) in a cell, such that the protein complex is translated into two proteins that independently fold. Example 2A linker peptides include F2A, P2A, E2A, and T2A, among others. Such peptides are generally 18-22 amino acids long, and can be derived from viruses.

In some embodiments, the actuator element 106 is associated with or connected to the effector element 110. In some embodiments, the exogenous polynucleotide sequence 101 includes the actuator element 106 associated with or connected to the effector element 110. The electrical-sensor element 102 can be associated with or connected either the effector element 110 or the actuator element 106. For example, the electrical-sensor element 102 can be located anywhere on the same plasmid as the effector element 110 and the actuator element 106, the electrical-sensor element 102 can be located on a different and separate plasmid which is or are used to genetically engineer the cell to form the ES effector cell 100. For example, the exogenous polynucleotide sequence 101 can include the actuator element 106 upstream from the effector element 110, and the effector element 110 upstream or downstream from the electrical-sensor element 102. In examples that include a (non-native) signal peptide 114, the signal peptide 114 is upstream from the effector protein 112.

FIG. 2 illustrates an example of a genetically engineered ES effector cell and a sequence of events triggered when in situ, in accordance with the present disclosure. The genetically engineered ES effector cell 200, herein generally referred to as “the ES effector cell 200” for ease of reference, can be used as or act as a living vector to synthesize the effector protein 212, as illustrated by the antiviral protein (AVP), using the artificial cell-signaling pathway and/or to trigger a sequence of events 220. The ES effector cell 200 synthesizes the effector protein 212 in situ upon interacting with electrical stimulation, as shown at 224.

As previously described, the ES effector cell 200 can comprise a polynucleotide sequence 201 including the electrical-sensor element encoding the CaV 204 that includes the pore-forming subunit 203 and the set of auxiliary subunits 207-1, 207-2, the actuator element 206 encoding at least one transcription factor binding site (e.g., NFAT), and the effector element 210 encoding the effector protein 212 and, optionally, the signal peptide 214. The ES effector cell 200 can comprise a single plasmid (e.g., a single construct including each of) comprising three constant domains (e.g., the actuator element 206, the signal peptide 214, and the electrical-sensor element), and a variable domain (e.g., the effector protein 212) arranged in cis. In other embodiments, ES effector cell 200 comprises multiple plasmids, such as a first plasmid comprising the actuator element 206 and the effector element 210, and a second plasmid comprising the electrical-sensor element.

The constant domains can be configured to provide functionality to the ES effector cell 200. The constant domains form part of the intracellular signaling pathway and includes the CaV 204 that mobilizes the calcium-dependent transcriptional machinery (e.g., actuator element 206) to upregulate the effector protein 212 which can optionally be fused to the signal peptide 214 that assists in transporting the effector transgene into the extracellular space 223.

The variable domain can be responsible for the applicability of the ES effector cell 200 to a variety of different diseases, target cells, therapy, and/or other applications. For example, the variable domain can impart therapy or other application specificity to the ES effector cell 200. In some embodiments, the effector protein 212 be exchanged or revised to reprogram the ES effector cell 200 with different therapeutic transgenes, such as for neutralizing the pathology that activated the ES effector cell 200 and essentially creating an off-shelf living vector.

FIG. 2 illustrates a specific example cell 200 engineered to include the electrically responsive artificial signaling pathway for Type-I IFN-β1 expression (ES effector cell with an IFN-β). In some examples, a K562 cell line is modified to form the ES effector cell 200. Referring to FIG. 2, the cell is engineered to include the CaV 204. The CaV 204 can include a L-type voltage-gated Ca²⁺ channel, such as Cav1.2 or Cav1.1, and which is sometimes herein referred to as the pore-forming subunit, and auxiliary subunits 207-1, 207-2, such as α₂δ₁, β₂, or β₃. The auxiliary subunits 207-1, 207-2 increase Ca²⁺ influx by regulating the opening and closing of the channel and trafficking the channel to the cell surface.

In some examples, the electrical stimulation is used to activate IFN-based innate immunity evolved to foil viral attacks. The ES effector cell 200 that encodes an IFN can be used as a therapy and/or vaccine to circumvent the antigenic stimulation of pattern recognition receptors that activate the IFN signaling often hijacked by pathogenic viruses. Unlike current vaccine approaches that rely on viral antigen subunits to generate target-specific memory cells and antibodies, the ES effector cell 200 is a universal intervention that does not require modifications specific for each virus and exert pan-viral immunity by providing an alternative IFN-synthesis pathway. In some experimental embodiments, as further described below, a mammalian cell line was engineered with a voltage-sensitive artificial signaling pathway that, when electrically stimulated, recruits a Type-I IFN response to compensate for what many pathogenic viruses evolve to abrogate. The electrical stimulus triggers a non-natural pathway that viruses may not evolve to hijack and thus decouples IFN synthesis from the innate pathways to regulate the production. The approach represents a departure from the existing methods of direct IFN infusion to activate innate immunity.

As shown by FIG. 2, at 1, while resting and in the absence of electrical stimuli, at 222, the CaV 204 is in a closed state. At 2, electrical stimulation is provided proximal to the ES effector cell 200, which causes the CaV 204 to transition to an open state, as shown at 3. The open state causes an influx of Ca²⁺ triggered by the electrical stimulation, and acts as a second messenger system to activate NFAT transcription factors and traffics them to the nucleus to interact with the NFAT transcription factors of the actuator element 206 to activate the cell 200, as shown at 4. This mobilizes the transcriptional machinery of the cell 200 to synthesize the transgenic encoded effector protein 212, in this case, Type-I IFN-β1, which has shown clinical benefits in terms of suppressing. In response to the activation, the effector protein 212 is transcribed and translated by the NFAT, as shown at 5, and the signal peptide 214 is cleaved off and effector protein 212 is transported into the extracellular space 223, at 6. In some embodiments, the effector protein 212 can act upon cells or cause the action, such as killing viral infected cells.

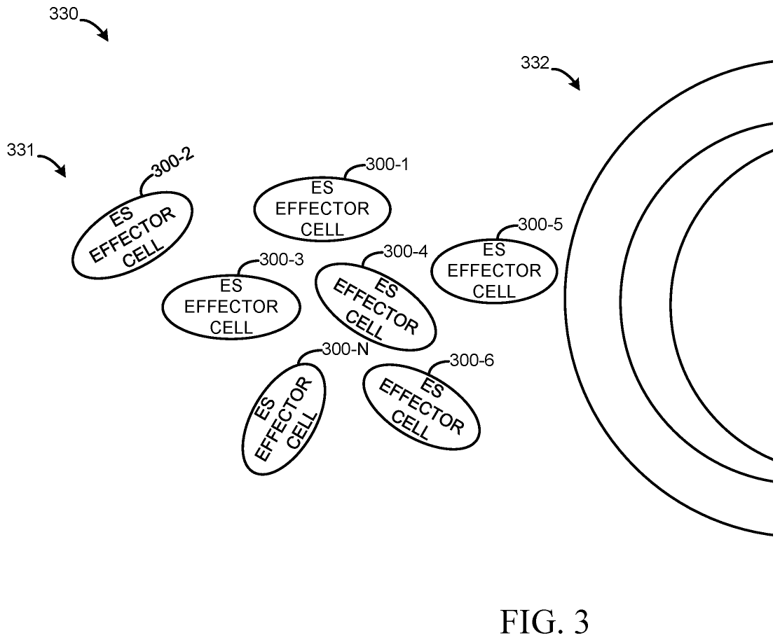

FIG. 3 illustrates an example of a population of genetically engineered ES effector cells, in accordance with the present disclosure. The population 331 can include a plurality of genetically engineered ES effector cells 300-1, 300-2, 300-3, 300-4, 300-5, 300-6, 300-N (herein generally referend to as “the ES effector cells 300” for ease of references). Each of the ES effector cells 300 can include at least substantially the same components and features as the ES effector cell 100 of FIG. 1, the details of which are not repeated for ease of reference.

In the example illustrated by FIG. 3, the environment is an extracellular space 330 that includes (a presence of) target cell(s), such that the space 330 can be referred to as a diseased environment. However, examples are not so limited and the environment can be associated with particular tissue, a body location, or other in situ environments in which electrical stimulation 332 may be applied. The population 331 of the ES effector cells 300 can be activated in response to the electrical stimulation 332. For example, in response to the electrical stimulation 332, the CaV of the ES effector cells 300 can transition to an open state, and in response, activate the ES effector cells 300 to synthesize the effector protein. In some examples, the ES effector cells 300 can provide a calibrated amount of the effector protein. For example, the calibrated amount of the effector protein can be a function of the electrical stimulation applied and a duration of the electrical stimulation applied in an extracellular space 330 or in a sample. Although the extracellular space 330 illustrates ES effector cells 300, the extracellular space 330 can further include a plurality of (host) cells, such as normal and/or diseased cells, among other non-cellular components.

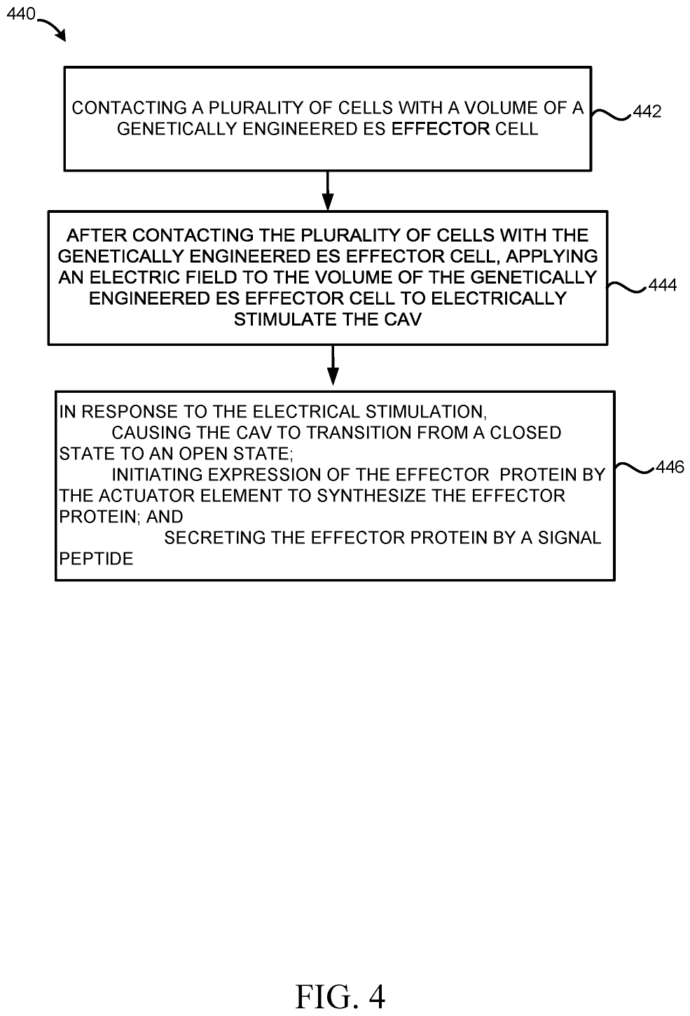

FIG. 4 illustrates an example method of contacting a plurality of cells with a volume of a genetically engineered ES effector cell, in accordance with the present disclosure. The method 440 can be implemented using the ES effector cell 100 illustrated by FIG. 1 and/or the population 331 of ES effector cells 300 illustrated by FIG. 3.

At 442, the method 440 includes contacting a plurality of cells with a volume of a genetically engineered ES effector cell. The cells can be contacted by contacting a sample with or administering the volume of the genetically engineered ES effector cell to a host, such as a patient. The genetically engineered ES effector cell can include at least some of substantially the same components and features as previously described by the ES effector cell 100 of FIG. 1, the details of which are not repeated for ease of reference. For example and as previously described, the ES effector cell can include an electrical-sensor element that encodes a CaV including a pore-forming subunit and a set of auxiliary subunits, an actuator element that encodes a transcription factor binding site, and an effector element that encodes an effector protein.

At 444, in response to contacting the plurality of cells with the volume of the genetically engineered ES effector cell, the method 440 includes applying an electric field to the volume of the genetically engineered effector cell to electrically stimulate the CaV. In some embodiments, the electric field can include an exogenous electric field which is applied using electrical circuitry. The electrical circuitry can include an external power supply and two (or more) electrodes coupled to the power supply. The method 440 can further include locating the electrical circuitry including the two electrodes proximal to the volume of the genetically engineered ES cell and applying the electric field by applying a voltage between the two electrodes using the power supply.

At 446, in response to the electrical stimulation, the method 440 includes causing the CaV to transition from a closed state to an open state, initiating expression of (e.g., transcription and translation of) the effector protein by the actuator element, and secreting the effector protein via a signal peptide. The effector protein can be secreted via a native or non-native signal peptide, as previously described. In response to the CaV transitioning to the open state, the method 440 includes causing an influx of Ca⁺² which activates the transcription factor binding site (e.g., a nuclear factor) and causes the expression of the effector protein.

In some embodiments, an amount of the secreted effector protein can be provided as a function of the electric field applied to the genetically engineered effector cell by the electrical stimulation and a duration of the electrical stimulation applied. For example, the genetically modified ES cell can be activated as a function of at least one of: a voltage, a total duration, a pulse duration, and a frequency of the electrical stimulation, with the activation being associated with the CaV being in an open state.

Various embodiments are directed to a pharmaceutical composition comprising a genetically engineered ES effector cell and a pharmaceutically acceptable carrier or excipient, such as the ES effector cell 100 of FIG. 1 and/or the population 331 of ES effector cells 300 of FIG. 3.

For example, an ES effector cell composition, such as a pharmaceutical composition, can comprises a plurality of the genetically engineered ES effector cells described herein and an acceptable carrier, diluents, or excipient (e.g., a pharmaceutically acceptable carrier, diluent, excipient or a combination thereof). The means of making such a composition have been described in the art (see, for instance, Remington's Pharmaceutical Sciences, 16th Ed., Mack, ed. (1980)). Preferably, the composition is prepared to facilitate the administration of the ES effector cells into a living organism. In some embodiments, the pharmaceutical composition comprises a plurality of ES effector cells as described herein and, for example, a balanced salt solution, preferably Hanks' balanced salt solution, or normal saline.

Some embodiments are directed to methods of forming the ES effector cells, such as genetically engineering or modifying a living cell to include the components and features as described by the ES effector cell 100 of FIG. 1.

The genetically engineered ES effector cells and cell compositions provided herein have properties advantageous for use in a variety of in vitro, ex vivo, and in vivo applications. For example, in vitro uses of the ES effector cells and cell compositions provided herein include, without limitation, research.

In vivo applications of the genetically engineered ES effector cells and cell compositions provided herein include, without limitation, in vivo imaging of disease sites, in vivo methods for localized therapy at a disease site (e.g., targeted therapy for ovarian cancer) or site of pathogen infection (e.g., targeted therapy for cells infected by dengue virus, Zika virus, West Nile virus, yellow fever, HIV, or a hepatitis virus (e.g., HepB, HepC)).

Various embodiments are directed to a panel of different types of genetically engineered ES effector cells, such as a plurality of ES effector cells engineered with different effector proteins, and which are used to simultaneously secrete different effector proteins.

Some embodiments are directed to methods of treating or preventing a disease using genetically engineered ES effector cells expressing a CaV and a therapeutic protein. For example, provided herein are methods comprising administering a genetically engineered ES effector cell expressing the CaV and the therapeutic protein. The disease against which the ES effector cell expressing the CaV is administered is not particularly limited as the ES effector cell is activated by electrical stimulus. Examples of the disease include a cancer (e.g., blood cancer (leukemia), solid tumor), an inflammatory disease/autoimmune disease (e.g., asthma, eczema), hepatitis, and an infectious disease, the cause of which is a virus such as Zika virus, SARS-CoV-1, SARS-CoV-2, influenza, and HIV, a bacterium, or a fungus, for example, tuberculosis, methicillin-resistant Staphylococcus aureus (MRSA), vancomycin-resistant enterococci (VRE), and deep mycosis. In some embodiments, a genetically engineered ES effector cell expressing the therapeutic protein can be used to decreased or eliminate target cells for treatment of the aforementioned diseases, that is, a tumor antigen, a viral antigen, a bacterial antigen or the like, is administered to treat or prevent such diseases. The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, methods described herein can provide any amount of any level of treatment or prevention of cancer in a mammal. Furthermore, the treatment or prevention provided by example methods can include treatment or prevention of one or more conditions or symptoms of the disease, e.g., cancer, being treated or prevented. Also, for purposes herein, “prevention” can encompass delaying the onset of the disease, or a symptom or condition thereof.

In some embodiments, genetically engineered ES effector cells are administered to a host (e.g., subject) in need thereof as a composition comprising the genetically engineered ES effector cells and a suitable carrier, diluent, or excipient as described herein. Any appropriate method of providing modified CaV-expressing cells to a host can be used for methods described herein. In some embodiments, methods for providing ES effector cells to a host can be adapted from clinical protocols for cellular and adoptive immunotherapy for infusion of donor-derived immune cells into a human host. In some embodiments, an adapted clinical protocol suitable for methods provided herein comprises obtaining ES effector cells from a host, genetically engineering (e.g., modifying) ES effector cells to express a CaV and NFAT-RE regulated protein transgene as described herein, and infusing the genetically engineered ES effector cells back into the host. A host, as used herein, includes and/or refers to any organism, such as a human, an animal (e.g., mammal, reptile, bird), insect, plant, among others, and which can be a subject of a study or test and/or a patient. A “subject” is sometimes interchangeably used with “host”. Host cells include cells obtained from the host.

Administration of the genetically engineered ES effector cells provided herein can be administered by any appropriate route, including, without limitation, administration intravenously, intratumorally, intramuscularly, subcutaneously, intraperitoneally, intra-arterially, or into an afferent lymph vessel, by parenteral administration, for example, by injection or infusion. In some embodiments, where genetically engineered ES effector cells or populations of such ES effector cells are administered, the ES effector cells can be cells that are allogeneic or autologous to the host, such as a mammal. Preferably, the ES effector cells are autologous to the host.

In some embodiments, a host to which genetically engineered ES effector cells are provided is monitored or assessed for increased (e.g., improved, more robust) tumor clearance or for other monitoring which is triggered by the electrical stimulation which causes expression of a detectable reporter protein. Various embodiments are directed to methods used for cancer therapies. In some embodiments, a host to which genetically engineered ES effector cells are provided is monitored or assessed for clearance of cells, where the detectable reporter protein can be configured to bind to target cells.

Some embodiments are directed to a method for cell-based treatment or prevention against a pathogen of interest. For such methods, the genetically engineered ES effector cell comprises a polynucleotide sequence encoding a therapeutic protein place of, or in addition to, the polynucleotide sequence encoding the detectable reporter protein; and is fused with a signal peptide (sec) on the 3′ end of the polynucleotide sequence to assist in extracellular transport. Upon triggering the cascade ES effector cell activation events and activation of the NFAT-RE, expression of a therapeutic protein is induced. The method can include the localized production of a therapeutic protein at the site of the target cell (e.g., a tumor cell, infected cell) and extracellular secretion of the therapeutic protein in the disease microenvironment.

Additional applications of the genetically engineered detectable reporter protein cells described herein include the following:

To target anticancer chemotherapeutic prodrugs to a tumor location, genetically engineered ES effector cells can be loaded with enzymatically activatable prodrugs, where the drug-activating enzyme is synthesized in response to electrical stimulation, thus providing controlled transformation of the prodrug into its active form. In some embodiments, the prodrug cannot be loaded into the ES effector cells, and can be infused in multiple doses subsequent to the infusion of the ES effector cells. The prodrug can alternatively be bound to an imaging nanoparticle or other means of image-guided means of active drug delivery. Attaching the prodrug to an imaging nanoparticle or engineering the ES effector cells to express imaging transgenes enables the ES effector cells to guide appropriate staging of the patient in preparation of surgery and for visually identifying and/or imaging tumor margins to assist in cytoreductive surgery.

Some embodiments are directed to methods of localized delivery of a chemotherapeutic agent to a site of the disease (e.g., tumor mass, site of autoimmune disease) comprises contacting a genetically engineered ES effector cell to a host cell population, wherein the genetically engineered ES effector cell comprises (i) an exogenous polynucleotide sequence encoding a CaV; and (ii) a NFAT-RE operably linked to a polynucleotide sequence encoding an enzyme, wherein, in response to electrical stimulation, the NFAT-RE is activated to initiate expression of the enzyme, which acts on the prodrug predesigned to be activated by this enzyme and uses it membrane permeability due to its hydrophobicity to be released.

With regard to surgical interventions for treating cancer, genetically engineered ES effector cells can be used for visualizing and/or imaging tumor margins via the expression of detectable reporter protein such as fluorescent proteins (e.g., GFP, GFP variants) or bioluminescent enzymes (e.g., luciferase). For example, such ES effector cells can be used to mark tumor margins to aid in surgical excision and to identify any residual positive tumor margins.

In some embodiments, genetically engineered ES effector cells are used for non-invasive detection and imaging of tumors based on expression of an imaging enzyme (e.g., thymidine kinase is capable of trapping a radioactive probe or otherwise detectable probe; tyrosinase detected by photoacoustic imaging or magnetic resonance imaging) expressed in response to electrical stimulation.

In some embodiments, genetically engineered ES effector cells can be used to circumvent safety concerns associated with vaccines against flaviviruses. By way of example, antigenic diversity among the four different dengue virus serotypes is responsible for the lack of antibody-mediated immunity and allows for multiple sequential infections. Although antibodies are effective in primary infection, their sub-neutralizing level during the secondary infections has been found to exacerbate the hemorrhagic fever by activating the complement system against the large infected cell mass in acute-phase. Prior dengue infection has also been found to worsen Zika infection. The use of ES effector cells can circumvent these safety concerns with flaviviruses because the ES effector cells, as described herein, can be engineered to express an antiviral protein, from human or non-human or synthetic origin, in response to the electrical stimulation.

In some embodiments, the genetically engineered ES effector cells comprise a CaV transitions to an open state in response to electrical stimulation and a NFAT response element to induce expression of an antiviral protein. Such embodiments can be used for transfusion medicine to treat emerging pathogens (e.g., SARS-CoV-1, SARS-CoV-2, Zika, dengue, West Nile, Yellow Fever).

Other non-limiting example uses of the genetically engineered ES effector cells include: i) imaging of the location of disease microenvironments to assist in surgical resection or monitor disease progression/regression; ii) cytotoxicity to kill the disease cells; iii) proliferation to enhance T-cell persistence; iv) immune-stimulation to recruit other immune cells; v) chemokine to recruit other immune cells; vi) immunosuppression to create localized immunosuppressive microenvironment; and vii) regeneration to enhance tissue healing.

As used herein, a target cell (sometimes herein interchangeably referred to as a “target cell of a host”, “target cell of interest”, “a diseased cell”, or “a target disease cell”) includes and/or refers to a cell of interest associated with a living organism (e.g., a biological component of interest). The ES effector cell can be from a variety of different type of cells, such as human and non-human cells, and sometimes herein referred to as “the source”. As used herein, the terms “genetically modified” and “genetically engineered” are used interchangeably and include and/or refer to a prokaryotic or eukaryotic cell that includes an exogenous polynucleotide, regardless of the method used for insertion. In some embodiments, the ES effector cell is modified to comprise a non-naturally occurring nucleic acid molecule that is created or modified by the hand of man (e.g., using recombinant deoxyribonucleic acid (DNA) technology) or is derived from such a molecule (e.g., by transcription, translation, etc.). An EA effector cell that contains an exogenous, recombinant, synthetic, and/or otherwise modified polynucleotide is considered to be a genetically engineered ES effector cell.

“Nucleic acid”, as used herein, includes and/or refers to a “polynucleotide,” “oligonucleotide,” and “nucleic acid molecule,” and generally means a polymer of DNA or ribonucleic acid (RNA), which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it can be suitable in some instances, as discussed herein, for the nucleic acid to comprise one or more insertions, deletions, inversions, and/or substitutions. In some embodiments, the nucleic acid can encode additional amino acid sequences that do not affect the function of the CaV and polynucleotide and which may or may not be translated upon expression of the nucleic acid by a host cell.

Nucleic acids can be obtained using any suitable method, including those described by Maniatis et al., Molecular Cloning: A Laboratory Manual, Cold Spring Harbor, N.Y., pp. 280-281 (1982) and/or U.S. Patent Application Publication No. US2002/0190663, each of which are herein fully incorporated in their entireties for their teachings. Nucleic acids obtained from biological samples typically are fragmented to produce suitable fragments for analysis.

Nucleic acids and/or other moieties can be isolated. As used herein, “isolated” includes and/or refers to separate from at least some of the components with which it is usually associated whether it is derived from a naturally occurring source or made synthetically, in whole or in part. Nucleic acids and/or other moieties of the invention can be purified. As used herein, “purified” includes and/or refers s separate from the majority of other compounds or entities. A compound or moiety can be partially purified or substantially purified. Purity can be denoted by a weight by weight measure and can be determined using a variety of analytical techniques such as but not limited to mass spectrometry, HPLC, etc.

### Experimental Embodiments

A number of experimental embodiments were conducted to generate genetically engineered ES effector cells and to characterize the ES effector cells functionality. Example constructs used to generate genetically engineered ES effector cells include the nucleotide sequences set forth in SEQ ID NOs: 1-17. SEQ ID NOs: 1-17 are each synthetic DNA. For example, SEQ ID NOs: 11-17 include example plasmid sequences used to generate ES effector cells, and provide example electrical-sensor elements, actuator elements, and effector elements. SEQ ID NOs: 1-2 provide example sequences for encoding pore-forming subunits and SEQ ID NOs: 3-5 provide example sequences for encoding auxiliary subunits. SEQ ID NOs: 6-10 provide example sequences for encoding effector proteins.

Various experimental embodiments were directed to form ES effector cells that express IFN, such as Type-I IFN and Type-III IFN and assessing the prophylactic and therapeutic effects of Type-I IFN and Type-IIII IFN produced from the ES effector cell with an IFN-β on SARS-CoV-2-infected cells. To mitigate potential oncogenesis-related risk upon ES effector cell infusion, radiation dose (γ-radiation) that renders the cell non-proliferative without compromising its capacity to produce IFNs was explored. This presents a translational approach as it ensures safe administration of the ES effector cell with the IFN-β that can be manufactured in compliance with current Good Manufacturing Practices (cGMP) for Phase I/II clinical trials. After assessing using the reporter enzyme, various experiments were directed to generating ES effector cells for IFN production and assessing the antiviral therapeutic effect of IFNs on SARS-CoV-2 infected Calu-3 cells.

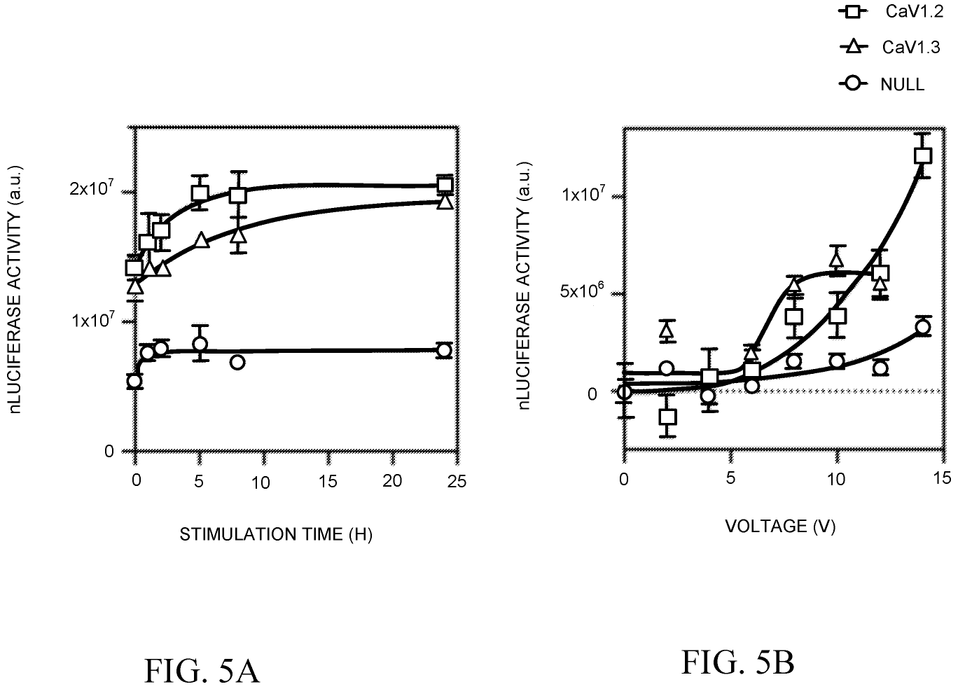

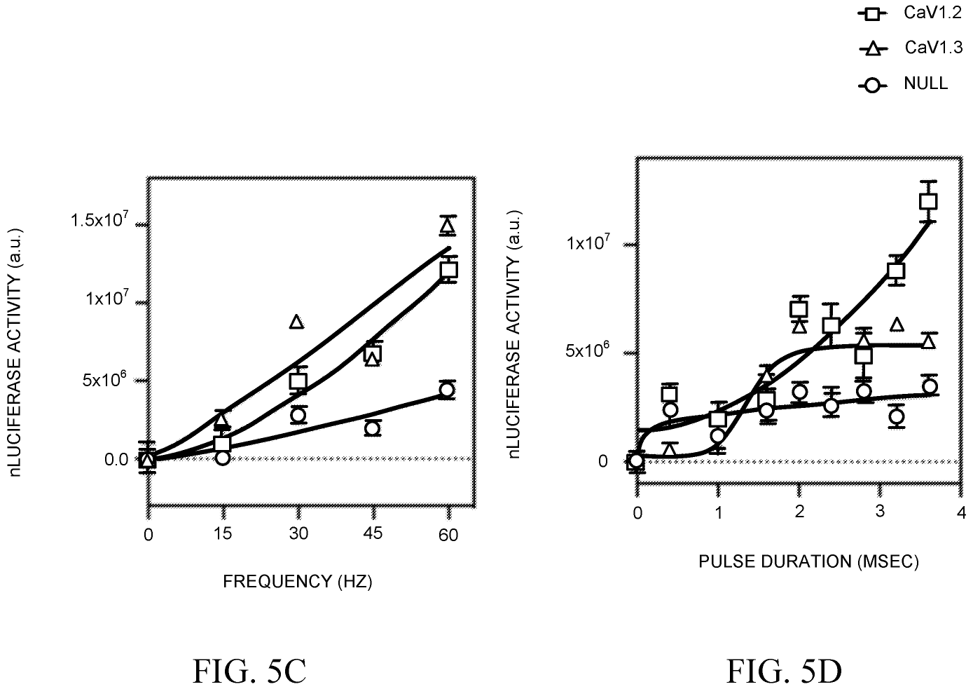

FIGS. 5A-5D illustrate example plots characterizing activation of genetically engineered ES effector cells by electrical stimulation, in accordance with the present disclosure. In some experimental embodiments, a reporter protein (nLuciferase) expression was used for assessing the output of the ES effector cell in context of sensitivity and off-target activation. Two different ES effector cells were generated. The first ES effector cell was modified to include an effector element encoding an effector protein of nLuciference, an actuator element encoding six NFAT-REs, and an electrical-sensor element encoding a CaV including CaV1.2 and auxiliary subunits of α₂δ₁, β₃, generally referred to as “the CaV1.2 cell” for ease of reference. The second ES effector cell was modified to include an effector element encoding an effector protein of nLuciference, an actuator element encoding six NFAT-REs, and an electrical-sensor element encoding a CaV including CaV1.3 and auxiliary subunits α₂δ₁, β₃, generally referred to as “the CaV1.3 cell” for ease of reference. A control, Null, was used that included a cell with no electrical-sensor element. FIG. 5A shows activations of the CaV1.2 and CaV1.3 cells by electrical stimulation as a function of stimulation time. FIG. 5B shows activations of the CaV1.2 and CaV1.3 cells by electrical stimulation as a function of voltage applied. FIG. 5C shows activations of the CaV1.2 and CaV1.3 cells by electrical stimulation as a function of frequency of the electrical stimulation applied. FIG. 5D shows activations of the CaV1.2 and CaV1.3 cells by electrical stimulation as a function of pulse duration. For FIGS. 5B-5D, reporter expression was background-subtracted to the unstimulated signal to correct for leaky reporter expression. Unless otherwise indicated, the cells were stimulated at 10 voltage (V) and 40 hertz (Hz) with a pulse duration 2 msec for 16 hours.

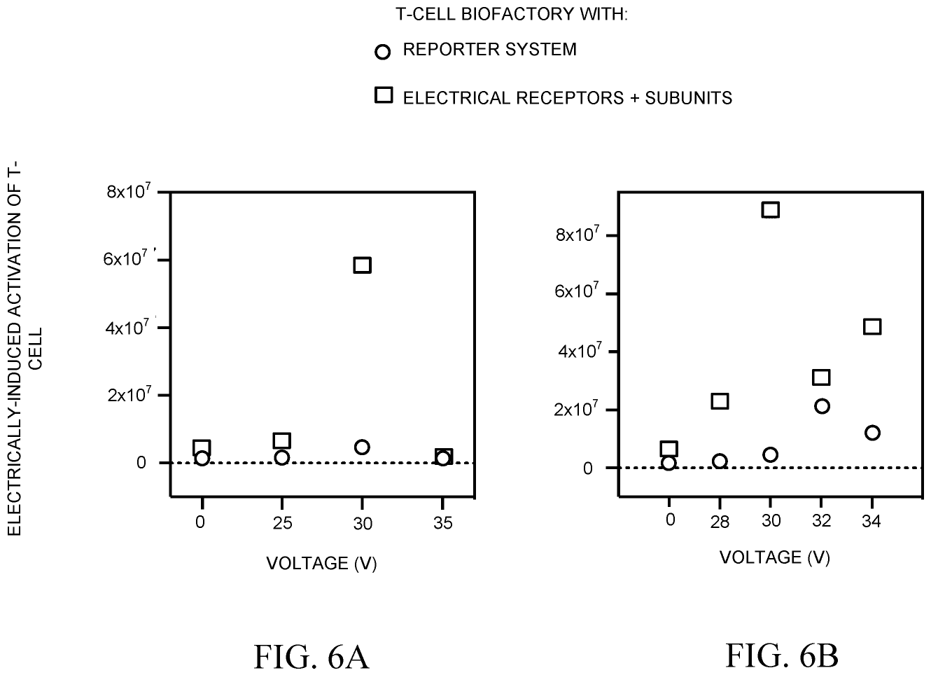

FIGS. 6A-6B illustrate example plots characterizing activation of a genetically engineered ES effector cell as compared to a reporter cell, in accordance with the present disclosure. As with FIGS. 5A-5B, in various experiments, T-cells were modified to include an electrical-sensor element encoding CaV1.2 or CaV1.3 and auxiliary subunits, an effector element encoding a reporter protein, an actuator element comprising six NFAT-REs. A control cell was generated which encoded the reporter protein without the electrical-sensor element. As shown by FIGS. 6A-6B, the T-cells modified to include the electrical-sensor element exhibited greater expression of the reporter protein than the control line at different V of electrical stimulation.

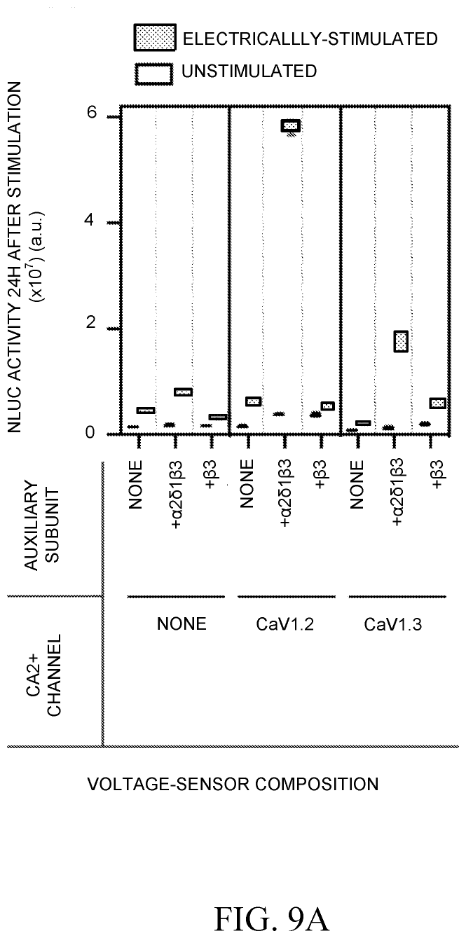

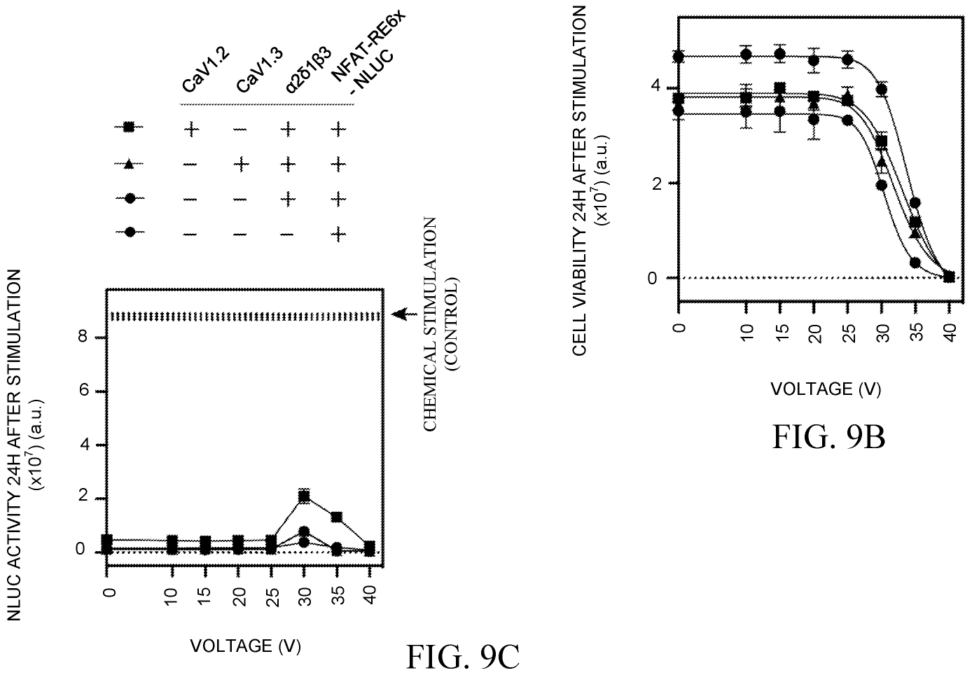

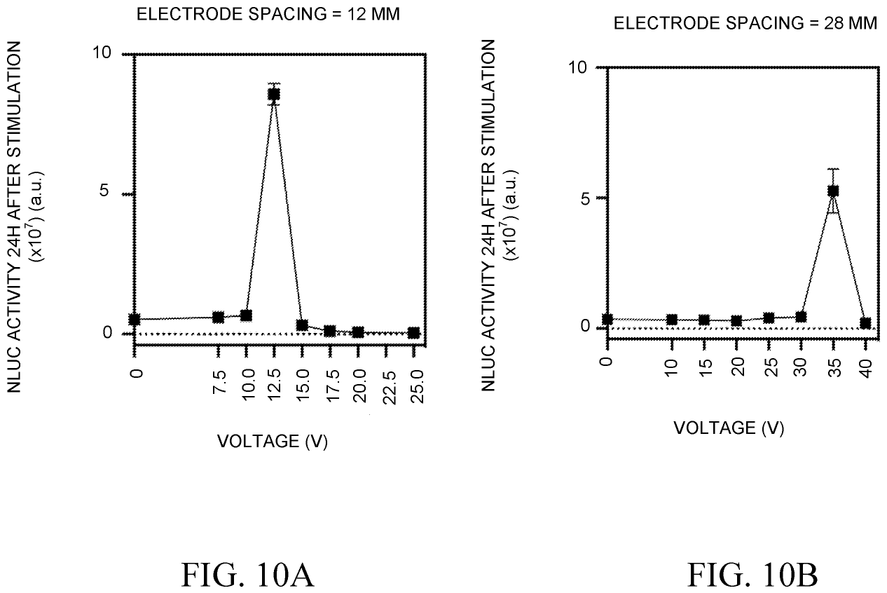

Further experiments were conducted to show the use and to define the electrical-sensor element components along with the voltage range to activate or trigger the ES effector cell, such as further illustrated by FIGS. 9A-9C. Co-expression of the complete set of auxiliary subunits (α₂δ₁, β₃) that mediate Ca²⁺ signaling in lymphocytes was used to implement the CaV channel (CaV1.2, CaV1.3) for sensing the applied voltage, as shown by FIG. 9A. Reporter protein (NanoLuc® (Nluc)) expression was used as a surrogate marker (ES effector cell with Nluc) for the effector protein and can be exchanged with a therapeutic protein against a specific disease indication, among other types of proteins. Initial experiments indicated that although some compromise in cell viability was observed (see FIG. 9B), a threshold value of 30 V triggered the ES effector cell for effector protein expression, as shown by FIG. 9C. The frequency and pulse duration during the initial experiments were kept constant at 20 Hz and 2 msec. The electrical activation of the ES effector cell to synthesize the effector protein was a function of the applied electric field, which can be defined as Applied Voltage÷Electrode Spacing, rather than applied V. This was confirmed by comparing the V to trigger the cells when placed between two electrodes with different spacing. Additional experiments showed about 2.5× higher Vis needed for 28 mm electrode spacing (6-well plate) when compared to 12 mm (24-well plate) (See FIGS. 10A-10B). Further experiments were conducted using a 6-well plate.

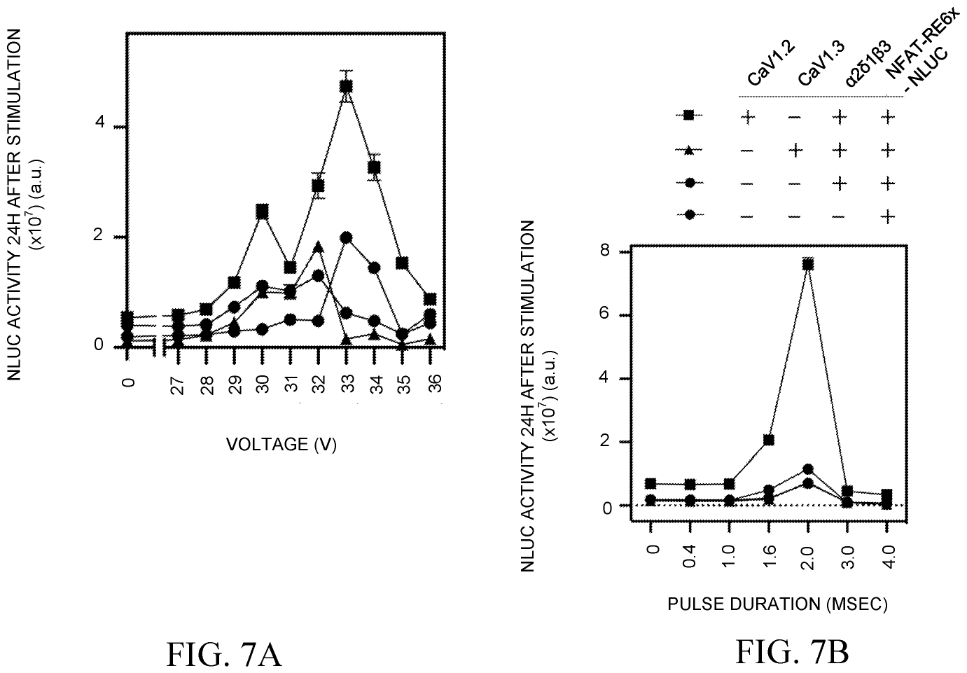

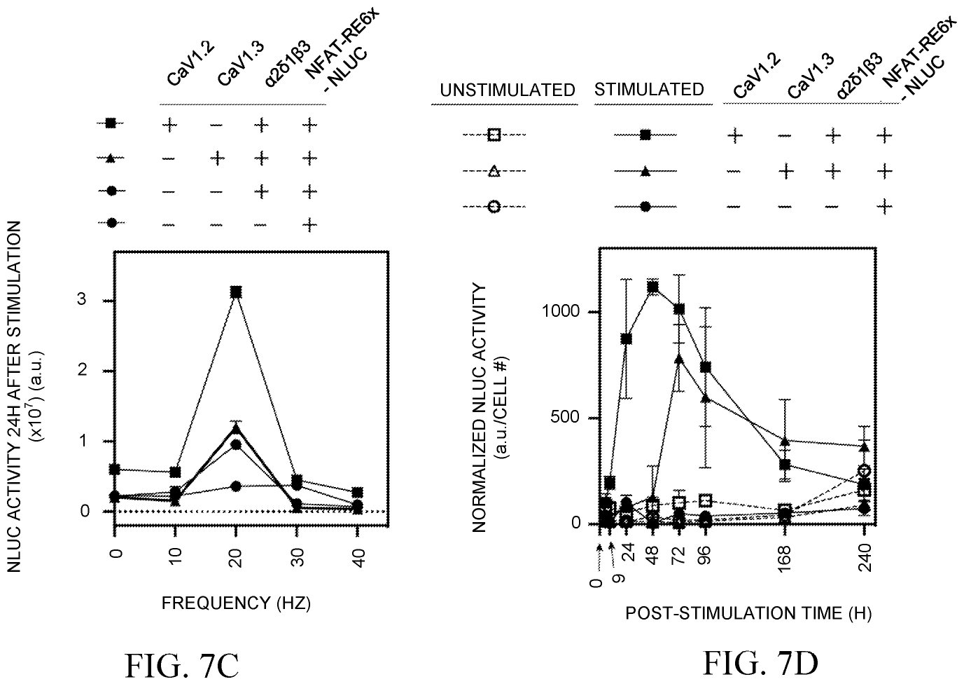

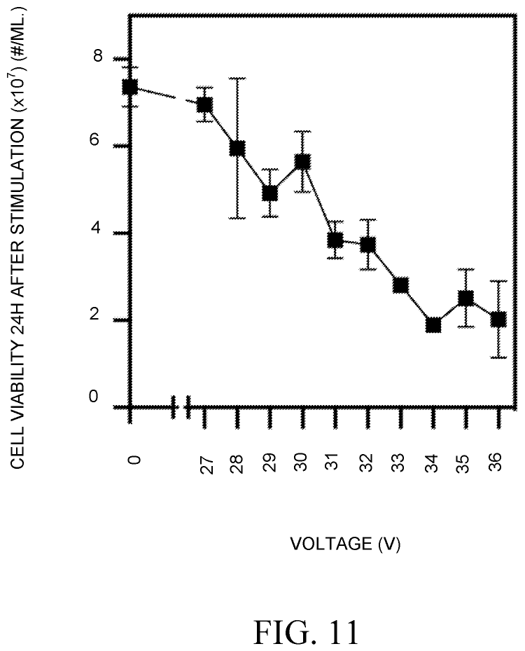

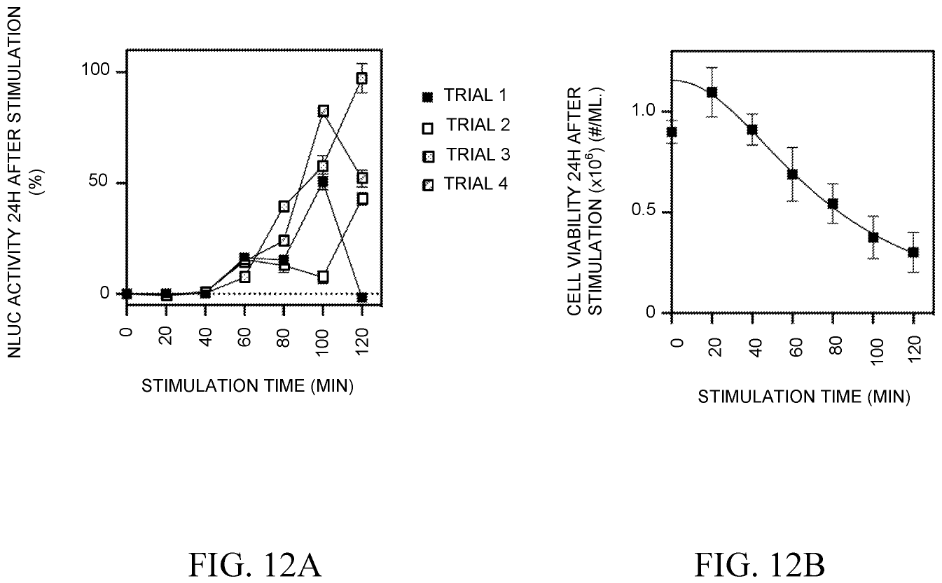

FIGS. 7A-7D illustrate example electrical parameters used to drive expression of the effector protein from a genetically engineered ES effector cell, in accordance with the present disclosure. Various experiments were used to identify electrical parameters to activate and/or trigger the ES effector cell and to select from among two different voltage-gated Ca²⁺ channels or pore-forming subunits (Cav1.2, Cav1.3) for the electrical-sensor element. FIG. 7A shows representative data with the x-axis depicting the applied voltage at a resolution of one V. The data shows increasing effector protein expression with rising voltage between 29 V and 33 V and the trend is comparable to what was observed with voltage-dependent gating of Cav1.2 and Cav1.3 channels. Effector protein expression declined beyond a certain applied V value, which was around 33 V. Investigations revealed the loss in cell viability at higher V as a potential cause (See FIG. 11). Additional experiments disclosed 30 V applied for 60 minutes (applied at 20 Hz frequency and 2 msec pulse duration) as an optimal stimulus. This was based on the minimal variability observed in the effector protein expression normalized to the number of live cells (See FIG. 12A) without significantly compromising the cell viability (See FIG. 12B) when stimulated within a range of electrical parameters. An iteratively conducted set of experiments informed on the range of different process parameters that can be used to electrically induce optimal effector protein expression.

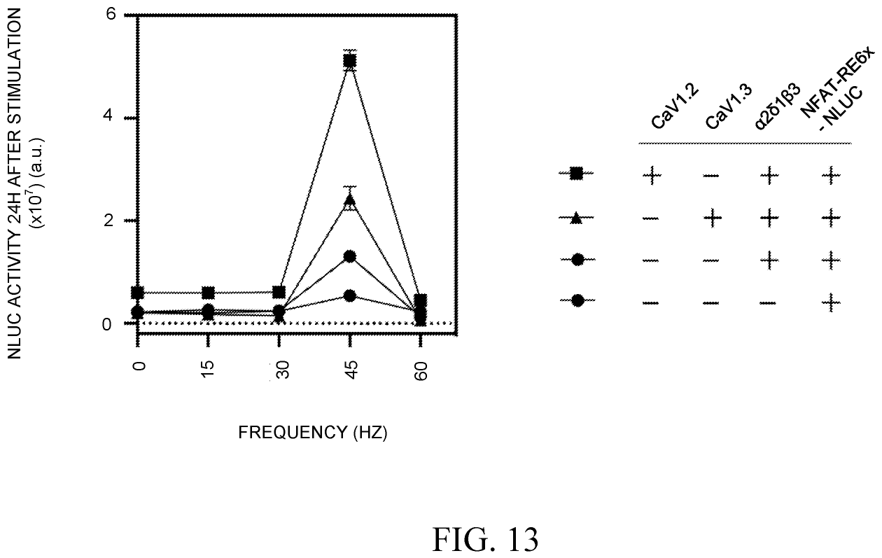

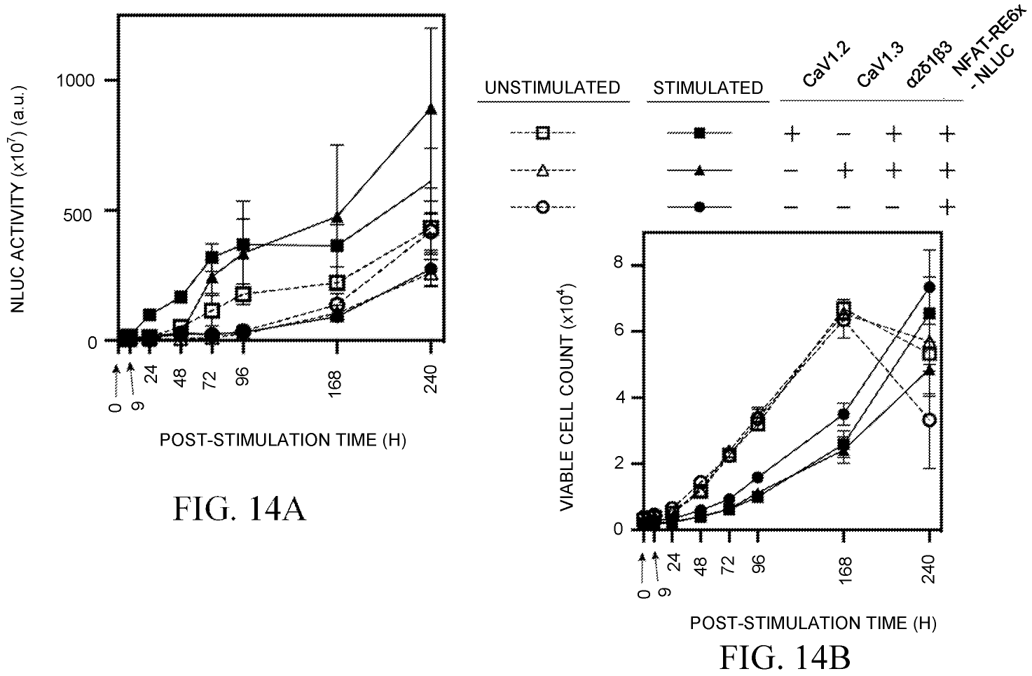

Representative data demonstrating the relationship of pulse duration and frequency to drive effector protein synthesis are shown in FIG. 7B and FIG. 7C respectively. At 30 V, a pulse duration of 2 msec and frequency of 20 Hz were found to optimally trigger the ES effector cell. The V to stimulate the ES effector cell for effector protein expression decreased with increasing stimulation frequency. The ES effector cells were treated at 20 V with a pulse duration of 2 msec, required a frequency of 45 Hz to activate effector expression (See FIG. 13). Stimulating the ES effector cell for 1 hour triggered effector protein expression that was detected within 9 hours, for the CaV1.2-based electric-sensor element, and increased over 48 hours and is represented in FIG. 7D. The effector protein expression normalized to the number of live cells after the duration depicted on the x-axis (see FIGS. 14A-14B for non-normalized data). The diminished effector protein expression in the two negative controls that did not include CaVs may be explained due to the endogenous CaV expression in T-cells. The two negative controls included the actuator element induced effector protein expression with and without the auxiliary subunits.

The trend was for both CaVs (CaV1.2, CaV1.3) in the electrical-sensor elements of the voltage-sensitive artificial signaling pathway with respect to applied voltage, pulse duration, frequency and post-stimulation time was similar. Effector protein expression, in response to the same electrical stimulation, in the ES effector cells with CaV1.2 was more robust compared to that from CaV1.3. Based on the observations reported in all panels of FIGS. 7A-D, CaV1.2 was thus empirically selected as the voltage-gated Ca⁺² channel to make up the electrical-sensor element and was co-expressed with α₂δ₁β₃.

As described above, Jurkat cells were engineered with an NFAT-RE-induced effector element (Nluc) and with no electrical-sensor element or electrical-sensor elements encoding the am′ and (33 auxiliary subunits alone or combined with Cav1.2 or Cav1.3. Nluc activity was assayed 24 hours after 1 hour of electrical stimulation of 16,500 cells at 30 V, 20 Hz and a 2 msec pulse duration, unless indicated otherwise on the x-axis. Analyzing a range of voltage, pulse duration, and frequency demonstrated the range of parameters used to stimulate the Nluc activity via the Cav1.2, and temporal analysis of Nluc activity showed normalized effector protein expression was induced for up to 48 hours. Nluc response was normalized to the cell number to take cell growth into account. Nluc activity for all observations was determined using n=4, error bars indicate ±1 SD and can also be considered as one half-width of a 68% confidence interval for the mean.

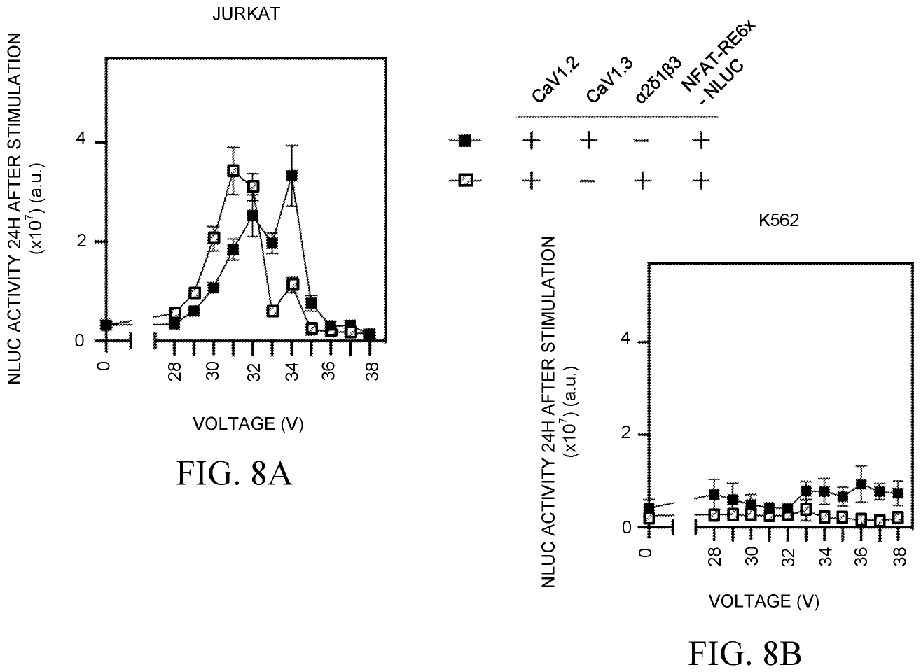

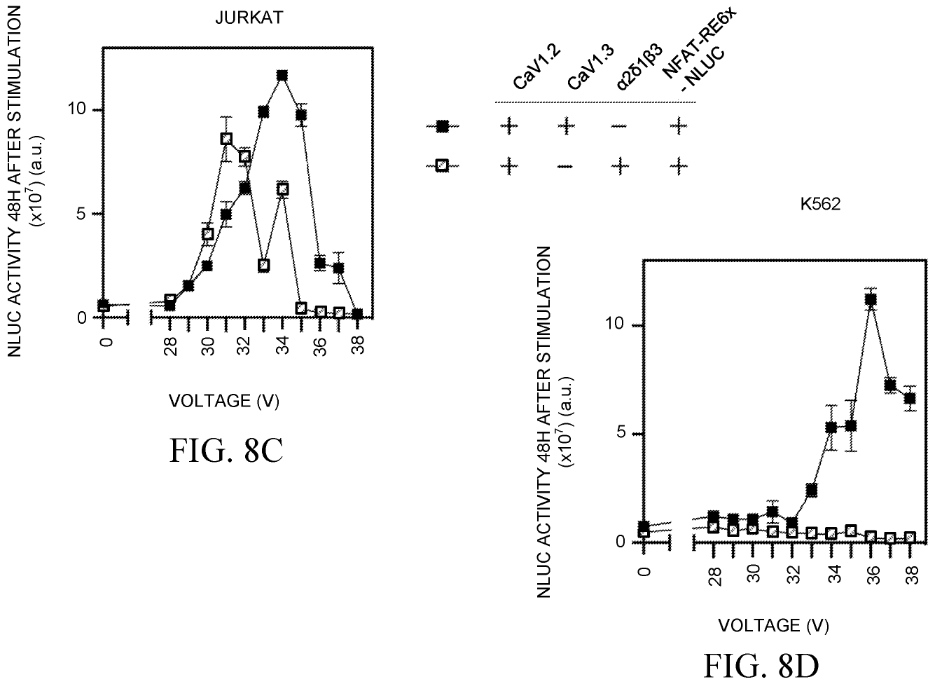

FIGS. 8A-8D illustrate example biological parameters used to drive expression of the effector protein from a genetically engineered ES effector cell, in accordance with the present disclosure. Various experiments were directed to assessing different biological parameters to determine regulation of the electrical-sensor element by the β isoform and the applicability of the synthetic circuit to function in other cell types. Such biological parameters included auxiliary subunit expression and the cell chassis.

FIGS. 8A-8B illustrate the Jurkat cell chassis used to generate a ES effector cell and show that in the Jurat cell chassis, β₃ and β₂ auxiliary subunits both supported electrical triggering of the electrical-sensor element to drive effector protein expression. FIGS. 8C-8D illustrates K562 cell chassis used to generate an ES effector cell and show that in the K562 cell chassis, β₂ auxiliary subunits supported electrical triggering of the electrical-sensor element to drive effector protein expression, and β₃ did not. Effector protein expression, when using Jurkat cell chassis was faster when compared to K562 cell chassis. For Jurkat cell chassis, it was detected within 24 hours and continue to increase at least until 48 hours. Comparatively, the effector protein expression was detected in K562 cells at 48 hours and was minimal at 24 hours. The experiments determined that the β₂ subunit better supported signaling by the electrical-sensor element in K562 cells modified to form ES effector cells, perhaps by increasing the trafficking of CaV1.2 to the plasma membrane. Channel localization at the cell surface can better drive Ca²⁺ influx through the CaV1.2 pore to provide the intracellular Ca²⁺ rise needed to translocate NFAT to the nucleus. These experiments demonstrate that effector protein expression can be electrically induced by the electrical-sensor element comprised of CaV1.2/α₂δ₁β₂ in Jurkat and K562 cells and can additionally extend to other cell chassis.

As shown by FIGS. 8A-8D, auxiliary subunit β₂ was compared to β₃ in supporting electrical activation of both Jurkat cell lines and K562 cell lines expressing Cav1.2/α₂δ₁ and NFAT-RE-induced effector protein (Nluc). Nluc activity was measured 24 and 48 hours after the initiation of 1 hour of electrical stimulation of 16,500 cells at 30 V, 20 Hz and a 2 msec pulse duration. Both β subunits promoted reporter expression in Jurkat cells. (32 only supported reporter expression in K562 cells. Nluc activity for all observations was determined using n=4, error bars indicate ±1 SD and can also be considered as one half-width of a 68% confidence interval for the mean.

In various experiments, the above-described ES effector cells are further modified to include effector elements encoding therapeutic proteins, such as an antiviral protein. There is rarely a perfect solution when it comes to implementing a public health response in a pandemic situation. Each virus manifests in a different manner, presents clinical symptoms that are vastly different, and has different molecular structure. A universal intervention that exerts pan-viral immunity, once approved by the Food and Drug Administration (FDA), promises to hold public trust for immediate deployment in the event of an outbreak. Various experiment demonstrate the ability to exogenously activate the engineered cells to trigger innate immunity against pathogenic viruses. Experiments demonstrated this feat by engineering cells that express IFN-(31 upon application of an electrical stimulus. Because the cell is triggered via an electrical stimulus, it does not require any prior knowledge about the virus in context of its molecular structure. The ES effector cell is broadly applicable and can be quickly deployed in case of any viral outbreak not limited to coronaviruses.

Almost all pathogenic viruses propagate by interfering with the IFN pathway, with early response in the prodromal stages to resist a viral attack. In various experimental embodiments, and at least for the above reason, IFN-(31, a Type-I IFN, was used as an effector protein from among the cells engineered to express different Type-I and Type-III IFNs known to exert antiviral effects in the preclinical and clinical trials. The experiments demonstrated similar IFN stimulated genes (ISGs) signatures upon treating the host cells with the IFN-β1 produced from the engineered ES effector cells compared to when treated with commercial IFN-β1 as a control. Due to the current public health crisis, SARS-CoV-2 was used as a model to represent emerging viruses. The impact of this work however goes beyond viruses. For example, IFN-β1 has been used for other diseases that otherwise evade the immune system, e.g. bacterial infections, autoimmune disorders, neurological diseases, and solid tumors. As previously described, the ES effector cell can accommodate any human or non-human protein, including recombinant version of IFN-β1 with therapeutic consideration. Site-specific synthesis of therapeutic proteins from an engineered cell stimulated exogenously from electrical circuitry, e.g., an extracorporeal device, therefore offers a capability for targeting many diseases.

The implementation of IFN-β1 in treating COVID-19 early during the infection is a testament to its demonstrated benefit previously observed in treating viral disease like hepatitis B and MERS. Formulations for direct systemic infusion of therapeutics e.g., liposomes, pegylation, hydrogels, microparticles, etc., have also been developed to improve efficacy and safety by reducing immunogenicity and improving bioavailability. Nevertheless, many challenges exist. For example (1) the two available mammalian cell- and bacterially-expressed formulations, IFN-β1a and IFN-β1b, exhibit systemic toxicities like inflammation, tissue damage, and multiorgan failure while the latter lacks appropriate post-translational modifications; (2) repeated dosing is still required for sustained bioavailability; and (3) temporal resolution with focused localized bioavailability that is needed for many treatments has not been achieved. To address these issues, cells were engineered for biologic production of proteins in situ. In some embodiments, the ES effector cell can include at least some of the components or features as described by Repellin C E, et al., entitled “Modular Antigen-Specific T-cell Biofactories for Calibrated In Vivo Synthesis of Engineered Proteins”, Advanced Biosystems, 2(12):1800210 (2018), and Repellin C E, et al, entitled “NK-Cell Biofactory as an Off-the-Shelf Cell-based Vector for Targeted In Situ Synthesis of Engineered Proteins”, Advanced BioSystems 5(7): 2000398 (2021), each of which are hereby incorporated in their entirety for their teaching, and which include an antigen-sensing chimeric antigen receptor (CAR) that, when engaging antigen-presenting target cells, mobilizes the Ca²⁺-induced second messenger system. To decouple activation triggers from in vivo signals, in experimental embodiments, the CAR molecule is replaced with an electrical-sensor element encoding a CaV that upon sensing the electric field mobilizes a downstream Ca²⁺-induced second messenger system. This activates the transcriptional machinery of the cell to activate the downstream pathway engineered to produce desired protein(s). Such ES effector cells localize the effector protein synthesis to the tissues in a regioselective manner thereby preventing harmful side effects of systemic infusion like inflammation and tissue damage.

The ES effector cell with IFN-β, a platform self-replicative cell-based antiviral, is capable of universally targeting most pathogenic viruses and offers to suppress an outbreak before it turns into a pandemic and extends the potential of its manufacturing in smaller decentralized bioreactors. It may therefore minimize the reliance on maintaining the cold chain, a major distribution hurdle for current vaccines that causes their spoilage. The ES effector cell-based antiviral approach bypasses the time- and resource-intensive processes that are integral for developing vaccines that introduces convalescent stage with specificity for each new pathogen, e.g., developing virus cultures, determining viral tropism, virus-antigen/host-receptor investigations, determining sequences, constructing and qualifying clinical grade immunogens, and iteratively conducting trials through design-build-test/safety cycles. The ES effector cell with IFN-β offers a new and complementary regimen, to current vaccine-based approaches, to treat emerging pandemics and, will be administered as an antecedent to treat infections at the prodromal stage.

FIGS. 9A-9C illustrate example results of genetically engineered ES effector cells with different pore-forming subunits and auxiliary subunits, in accordance with the present disclosure. Voltage-gated Ca²⁺ channels and auxiliary subunit engineering mediated electrical triggering of synthetic NFAT-regulated Nluc reporter expression was experimentally assed. CaV1.2 and CaV1.3 and α₂δ₁ and (33 auxiliary subunits were co-engineered into the Jurkat cell line with a Ca²⁺-inducible reporter protein. Nluc activity identified auxiliary subunit expression, as shown by FIG. 9A, and voltage requirements to trigger electrical-sensor activation of the NFAT actuator element, as shown by FIG. 9B. FIG. 9A illustrates co-expression of α₂δ₁β₃ with Cav1.2 and, to a lesser extent, CaV1.3 supported significant voltage-dependent effector protein expression. FIGS. 9B and 9C illustrate increasing voltage application activated reporter protein expression and reduced cell viability. NFAT-mediated transcription of the reporter protein expression was also induced with PMA/Ionomycin treatment (dashed lines in FIG. 9B). In all cases, the cells (16,500 k) were stimulated at 30 V (or as indicated in FIGS. 9B-9C), 20 Hz and a 2-msec pulse duration; Nluc activity was measured 24 hours post-stimulus. Nluc activity for all observations was determined using n=4, error bars indicate ±1 SD and can also be considered as one half-width of a 68% confidence interval for the mean.

FIGS. 10A-10B illustrate example results of using different electrode distances for electrically stimulating a genetically engineered ES effector cell, in accordance with the present disclosure. As shown, decreasing the inter-electrode distance reduced the voltage threshold for ES effector cell activation. Two Ion-optix C-pace lid configurations, having 12.5 mm spaced electrodes, as shown by FIG. 10A, and 28 mm spaced electrodes, as shown by FIG. 10B, were used to stimulate Cav1.2/α₂δ₁β₃-expressing Jurkat cells. Nluc activity was determined 24 hours after the post-initiation of electrical stimulation (1 hour, 30 V, 20 Hz and 2 msec). Decreasing the inter-electrode distance from 28 to 12.5 mm proportionately reduced the V to activate reporter protein expression. Nluc activity for all observations was determined using n=4, error bars indicate ±1 SD and can also be considered as one half-width of a 68% confidence interval for the mean.

FIG. 11 illustrates example cell viability as a function of voltage application for electrically stimulating a genetically engineered ES effector cell, in accordance with the present disclosure. As shown by FIG. 11, cell viability decreased with increasing voltage application. CaV1.2/a₂δ₁β₃-expressing Jurkat cells were counted 24 hour after initiation of exposure to the indicated voltage applied for 1 hour at 20 Hz and 2 msec pulse duration. Cell numbers were measured with Trypan Blue using n=2, error bars indicate ±1 SD and can also be considered as one half-width of a 68% confidence interval for the mean.

FIGS. 12A-12B illustrate example results of using different durations for electrically stimulating a genetically engineered ES effector cell, in accordance with the present disclosure. The duration of electrical stimulation impacts both effector protein expression activation and cell viability. CaV1.2/α₂δ₁β₃-expressing Jurkat cells were electrically stimulated (30 V, 20 Hz, 2 msec pulse duration) for the indicated time and analyzed, 24 hour after stimulus initiation for Nluc Effector activity, as shown by FIG. 12A, and AO/PI-determined cell viability, as shown by FIG. 12. Four trials demonstrated that 60 minutes of electrical stimulation was required to activate NFAT-mediated Nluc expression and that stimulation beyond this window enhanced activation but at the expense of reduced cell viability. Nluc activity for all observations was determined using n=4, error bars indicate ±1 SD and can also be considered as one half-width of a 68% confidence interval for that mean. Nluc activity of the four trials was normalized by setting 0% as the signal for untreated cells and 100% as the signal for PMA/Ionomycin-treated cells. Cell viability numbers represent the mean of AO/PI measurements obtained for the four trials, error bars indicate ±1 SD and can also be considered as one half-width of a 68% confidence interval for the mean.

FIG. 13 illustrates example results of using different frequencies for electrically stimulating genetically engineered ES effector cells, in accordance with the present disclosure. As shown by FIG. 13, stimulating frequency increases with decreasing voltage strength. The plot of FIG. 13 shows Nluc activity assayed reporter expression 24 hours after initiation of 1 hour of electrical stimulation of 16,500 cells at 20 V and a 2 msec pulse duration using the range of frequencies indicated on the x-axis. In comparison to cells stimulated at 30 V in FIG. 7C, where 20 Hz frequency yields reporter protein expression, stimulating CaV1.2-expressing cells at 20 V requires increased 45 Hz frequency to detect Nluc activity. Nluc activity for all observations was determined using n=4, error bars indicate ±1 SD and can also be considered as one half-width of a 68% confidence interval for the mean.

FIGS. 14A-14B illustrate example results of using different durations for electrically stimulating a genetically engineered ES effector cell, in accordance with the present disclosure. More particular, FIGS. 14A-14 B illustrate a duration of Nluc expression in ES effector cells. Cells were stimulated for 1 hour at 30 V, 20 Hz, and 2 msec pulse duration and Nluc activity, as shown by FIG. 14A, and viable cell count, as shown by FIG. 14B, were assayed at the indicated time. Significant reporter expression was observed in electrically-stimulated CaV1.2- and Cav1.3-expressing cells. Nluc activity also increased with time-dependent cell growth. Nluc activity was, thus, normalized to relative cell number and reported in FIG. 7D. Nluc and CellTiter-Glo activity for all observations was determined using n=4, error bars indicate ±1 SD and can also be considered as one half-width of a 68% confidence interval for that mean.

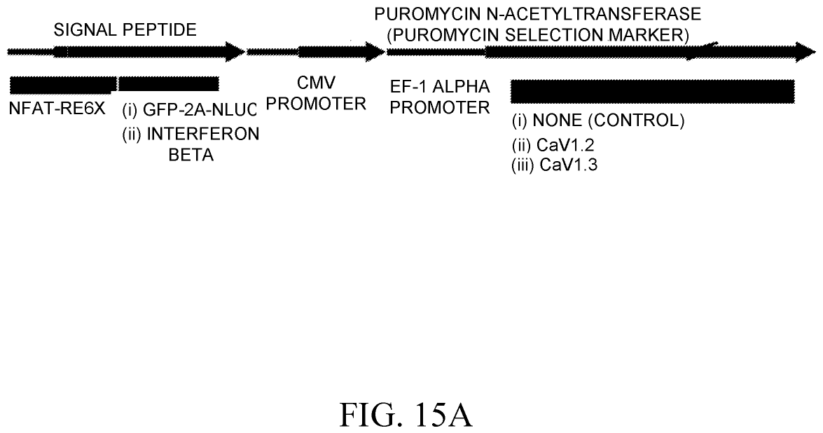

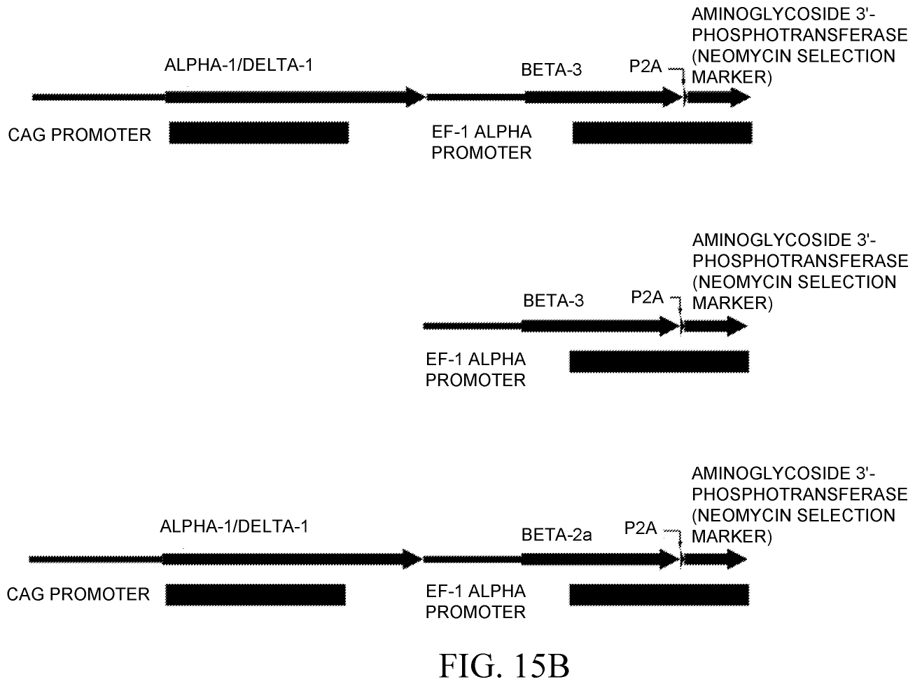

FIGS. 15A-15B illustrate example plasmids for generating genetically engineered ES effector cells, in accordance with the present disclosure. More particularly, FIG. 15A illustrates plasmid A and FIG. 15B illustrates plasmid B from Table 1 further described below.

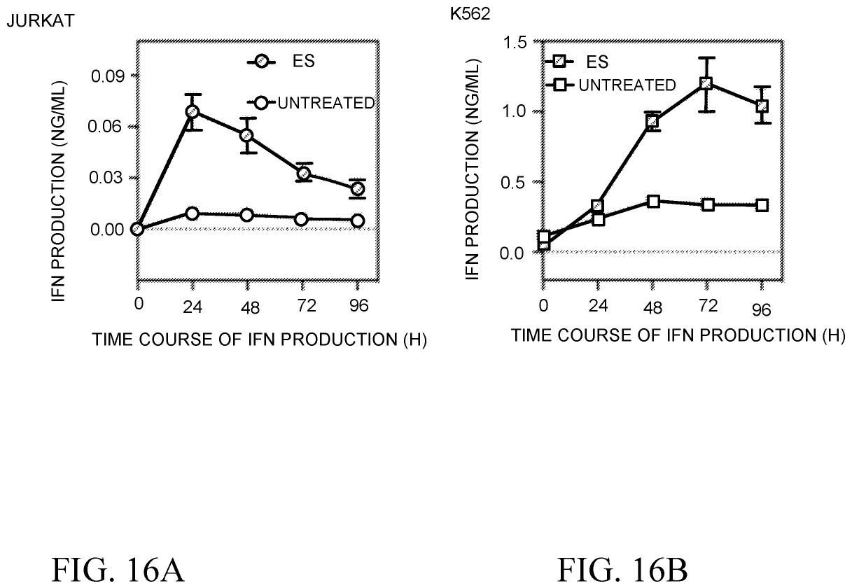

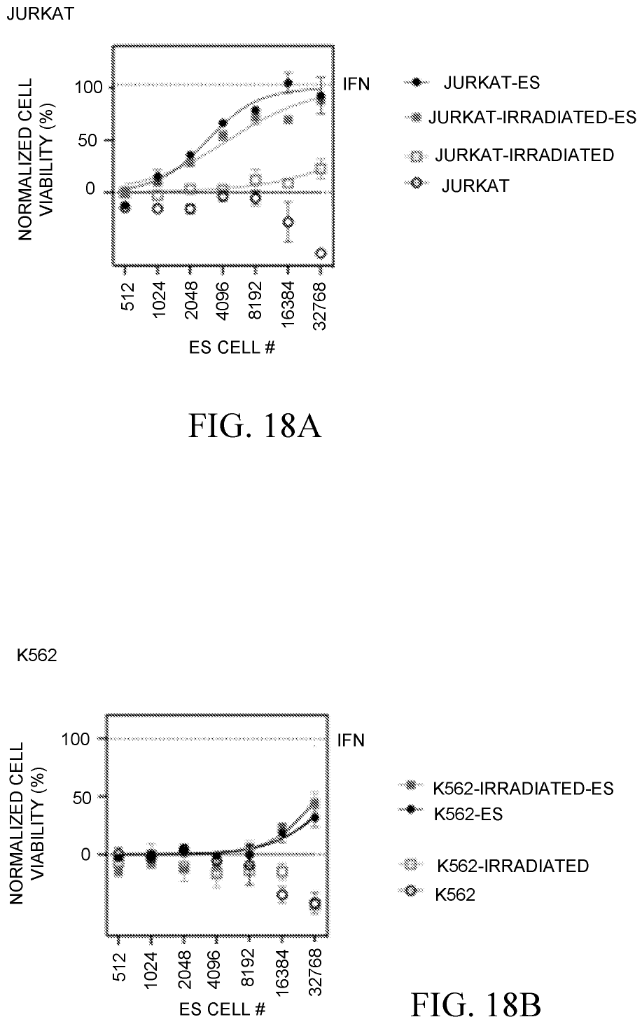

FIGS. 16A-16B illustrate example results of electrically stimulating genetically engineered ES effector cells, in accordance with the present disclosure. More particularly, FIGS. 16A-16B illustrates the Jurkat and K562 cell chassis both support electrically-stimulated IFN production. As observed with nLuciferase reporter expression kinetics, Jurkat cells produce IFN more rapidly with detection of the secreted cytokine within 24 h of stimulation (FIG. 16A). In contrast, IFN produced, in response to electrical activation, was observed 48 hours post-stimulation (FIG. 18B). Regardless, engineering the electrically-sensor into both cell lines enables on demand IFN production independent of disease biomarkers.

More particularly, FIGS. 16A-16B show time course of IFN secretion by the EE effector cell. Electrical parameters that drove nLuc reporter expression were used to trigger IFN secretion (1 hour stimulation at 30 V (Jurkat) and 34 V (K562), 2 msec pulse duration at 20 Hz). IFN production was determined by isolating the ES effector cell supernatant post-stimulation and using an IFN-sensing reporter cell line to quantify the secreted effector protein.

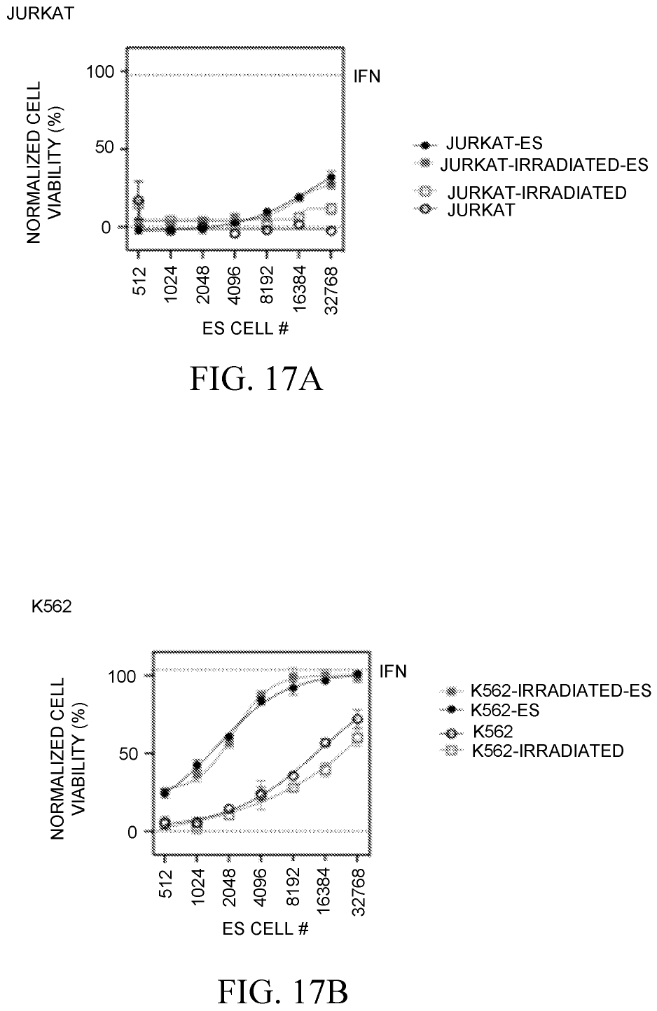

FIGS. 17A-17B illustrate example results of electrically stimulating genetically engineered ES effector cells, in accordance with the present disclosure. More particular, FIGS. 17A-17B shown IFN secreted by the ES effector cell prevents SARS-CoV-2 infection. IFN-containing supernatant isolated from electrically stimulated ES-Cell was used to pretreat Vero-E6 cells prior to SARS-CoV-2 infection to assess the prophylactic activity of secreted IFN. Within Jurkat and K562 cell lines (FIG. 16A and FIG. 16B, respectively), the ES effector cell that produces IFN circuit enabled IFN production in response to the electric field that significantly suppressed viral infection compared to the unstimulated ES effector cells. Treating the viral infection with IFN synthesized by the K562-based ES effector cell suppressed viral infection more effectively than that produced by Jurkat cells, which likely reflects the robust IFN production observed in K562 cells. These embodiments demonstrate use of remote-triggering to stimulate the ES effector cell and synthesize IFN on demand to treat viral infection. Moreover, utilizing the ES effector cell circuit to direct drug production in the K562 cell line (approved for clinical trials) paves the way for in vivo applications.

FIGS. 17A-17B show the supernatant of the ES effector cell (FIG. 17A: Jurkat and FIG. 18B: K562) was serially diluted and used to pretreat VeroE6 cells prior to SARS-CoV-2 infection to suppress viral replication and cell death. The supernatant taken 48 hours post treatment from electrically-stimulated ES effector cells, both irradiated and non-irradiated, was compared to that of unstimulated cells. The dashed blue line shows viral protection of purified, recombinant IFN.

FIGS. 18A-18B illustrate example results of electrically stimulating genetically engineered ES effector cells, in accordance with the present disclosure. As shown. IFN secreted by the ES effector cell suppresses active SARS-CoV-2 infection. The ES effector cells were cultured with freshly infected SARS-CoV-2-infected VeroE6-Luc 2 cells to determine the therapeutic effect of in situ IFN delivery on mitigating an ongoing viral infection. Within Jurkat- and K562-based ES-Cell (FIGS. 18A and 18B, respectively), the electrically-responsive circuit enabled IFN production that significantly suppressed an established viral infection compared to unstimulated ES effector cells. In contrast to the ES effector cell supernatant pretreatment study (FIGS. 17A-17B), the Jurkat-based ES effector cell that produces IFN markedly reduced a developing viral infection and its ensuing cell death compared to K562-based drug delivery. Given that Jurkat-based ES effector cells secreted IFN more rapidly after electrical stimulation (FIGS. 16A-16B), quicker IFN delivery seems to be more effective in suppressing active viral infection versus robust IFN expression demonstrated by the K562-based ES effector cell that seems to be more effective as a prophylaxis.

More particularly, FIGS. 18A-18B show ES effector cell (FIG. 18A: Jurkat and FIG. 18B: K562) were serially diluted and cocultured with VeroE6-Luc2 cells shortly after their infection with SARS-CoV-2. Luciferase activity of the VeroE6-Luc2 cells was determined 48 hours after infection/coculture to determine cell viability. Prior to co-culture, the ES effectorsells, both non-irradiated and irradiated, were either electrically stimulated. Unstimulated cells were used as negative controls. The dashed blue line shows viral protection of purified, recombinant IFN.

The following provides various materials and methods.

Materials and Reagents: Engineered Jurkat E6-1 (ATCC, Cat #TIB-152) and K562 (ATCC, Cat #MCCL-243) cell lines were maintained in complete RPMI media [RPMI1640 (Corning, Cat #10-041-CV)], 10% heat-inactivated fetal bovine serum (FBS) (Sigma-Aldrich, Cat #F2442-500ML) and 1× Penicillin-Streptomycin solution (Corning, Cat #30-002-C1)]. Engineered Vero-E6 cells (ATCC, Cat #CRL-1586) were cultured in complete Eagle's MEM [EMEM (Corning, Cat #10-009-CV)] supplemented with 10% heat-inactivated fetal bovine serum (FBS) (Sigma-Aldrich, Cat #F2442-500ML) and 1× Penicillin-Streptomycin solution (Corning, Cat #30-002-C1)]. All cells were expanded, and liquid nitrogen stocks were maintained using freezing media (50% FBS, 40% RPMI and 10% DMSO). Plasmids encoding different genetic payloads (transfer plasmids) were designed in SnapGene software (GSL Biotech LLC) and sub-cloned into piggyBac vector plasmid (System Biosciences, Cat #CD510B-1) or piggyBac Transposon vector plasmid (System Biosciences, Cat #PB510B-1). PiggyBac Transposase sequence was provided by the Johns Hopkins University School of Medicine as described by Doherty J E, et al. (2012); Hyperactive piggyBac gene transfer in human cells and in vivo. Human Gene Therapy 23, 311-320, which is hereby incorporated by reference in its entirety for its teaching. An insert for “EF1alpha promoter—i7pB transgene—bGH poly(A) signal” was chemically synthesized and assembled using overlapping PCR products into pUC19 (GenBank: L09137, New England Biolabs, #N3041). All plasmid preparation services (chemical synthesis of DNA insert sequences, sub-cloning into respective vector backbones, and the amplification) were obtained from Epoch Life Science, Inc. (Missouri City, TX). Jurkat and K562 cell line engineering used the respective nucleofection kits: SE Cell line 4D-Nucleofector™ X Kit S (Lonza Cat #V4XC-1032) and SF Cell line 4D-Nucleofector™ X Kit L (Lonza Cat #V4XC-2024), according to manufacture instructions. Puromycin dihydrochloride (ThermoFisher Scientific, Cat #A1113803) and G418 Sulfate (Corning, Cat #30-234-CI) were used for selecting stable cells. Nano-Glo® Luciferase Assay (Promega #N1150) was used to measure Nluc activity. Cell viability was either determined with CellTiter-Glo® 2.0 (Promega #G9242) or ViaStain™ AO/PI Cell Staining (Nexcelom Cat #CS2-0106). Vero-E6-Luc2⁺ cells were engineered as described and used as a reporter of cell killing by SARS-CoV-2. The SARS-CoV-2 virus culture [BEI Resources, NIH; Hong Kong/VM20001061/2020 (Cat #NR-52282)] was provided. Viability of Vero-E6-Luc2⁺ cells was assessed using either the CellTiter-Glog 2.0 Cell Viability Assay (Promega, Cat #PR-G7570) or the One-Glo® assay (Promega, Cat #E6110) for Luc2 activity. HEK-Blue IFN-α/(3 cells (InvivoGen, Cat #hkb-ifnab) were used for quantification of the amount of IFNs produced by the ES effector cell with IFN-β, following manufacturer's instructions, in complete DMEM (DMEM growth media (Corning, Cat #10-013-CV) supplemented with 10% heat-inactivated fetal bovine serum (FBS) (Sigma-Aldrich, Cat #F2442-500ML) and 1× Penicillin-Streptomycin solution (Corning, Cat #30-002-C1)). Recombinant human IFN-β1a protein from R&D systems (Cat #8499-IF-010/CF) was used as a control.

Generation of ES effector cells (ES effector cell with Nluc; ES effector cell with IFN-β). Jurkat and K562 suspension cell lines were engineered with the piggyBac Transposon system as previously described by Repellin et. al 2020, which is hereby incorporated herein in its entirety for its teaching. Transfer plasmids contained the following genetic components: CaV1.2: Q13936-20; CaV1.3: Q01668-1; α₂δ₁: P54289-2; β₃: P54284-1; β₂ₐ: Q08289-2; Nluc: GenBank: JQ437370.1, IFN-β1: P01574. Following nucleofection with the transfer plasmid and the piggyBac Transposon vector, the transfected cells were cultured for 48 hours and, then, placed in selection using 0.5 μg/mL of puromycin dihydrochloride or 1 mg/ml G418. Following selection, cells were expanded as required for different assays and frozen using freezing media. The ES effector cell with Nluc and ES effector cell with IFN-(3 required two rounds of nucleofection to introduce: Plasmid A) CaV1.2 or CaV1.3 along with the actuator and effector elements (puromycin-selected) and Plasmid B) the α₂δ₁ and β auxiliary subunits (G-418-selected). For irradiation, the ES effector cells were exposed to 30 Gy (or as indicated) using a ¹³⁷Cs γ-emitting irradiator, Mark I-68A (J L Shepherd and Associates) at a dose rate of 222 mGy/min. The control cells were treated similarly except for the irradiation.

Biphasic electric field stimulation of ES effector cells. Cells were cultured in either 6 well or 24 well dishes fitted with C-Dish™ lids (IonOptix Cat #CLD6WFC and CLD24WF) to immerse two carbon electrode elements within each well. The cells were electrically stimulated with the IonOptix C-Pace EM stimulator. In general experiments, 3 ml of cells were plated per well at the density of 0.17×10⁶ cells/ml. 24 hour-post plating, the cells were electrically stimulated for 1 hour and then assayed for effector protein expression 24 to 48 hours after the onset of electrical stimulus.

Nluc reporter assay. Effector protein expression was measured with the Nano-Glo® assay (Promega, Cat #N1120) according to manufacturer instructions. The Nluc substrate was diluted in cell lysis buffer and added 1:1 to treated cells transferred to a 96-well plate. Bioluminescence was read on a microplate reader (Perkin Elmer, EnVision™ Multilabel Plate Reader, Model: 2104-0010A).

IFN-β1 reporter assay. IFN-β1 production was determined using HEK-Blue IFN-α/β reporter cells. Secreted IFN was isolated by collecting the supernatant following centrifugation of the treated cells at 500 g for 5 min. Following manufacturer's instructions, 50 μl of the serially diluted supernatant was mixed with 150 μl 50,000 HEK-Blue IFN-α/β reporter cells in DMEM growth media and plated in a single well of a 96-well plate. Recombinant IFN-β1a, serially diluted in DMEM growth media, was plated in parallel to generate a standard curve for IFN concentration. After 24 hours of incubation, 20 μL of HEK-Blue IFN-α/(3 (or HEK-Blue IFN-2) supernatants was added to 180 μL of Quanti-blue substrate (InvivoGen), and incubated at 37° C. Absorbance was measured at 650 nm using microplate reader (Perkin Elmer, EnVision™ Multilabel Plate Reader).

Antiviral prophylactic effect of the ES effector cell with IFN-β. 24 hours after plating 1×10⁶ cells in 3 ml in complete RPMI media, ES effector cell with IFN-β were electrically stimulated for 1 hour with 2 msec, 30 V at 20 Hz. 48 hours post-stimulus initiation, the supernatant was isolated with 500 g centrifugation for 5 minutes and serially diluted with complete EMEM media. 100 μL of serially diluted cell supernatants were then used to pre-treat a monolayer of Vero-E6-Luc2+ cells for 24 hours (20,000 cells/well, triplicates) in a 96-well plate. Recombinant human IFN-β1a (1 μg/ml) was included as a positive control. The pre-treated cells were then infected with SARS-CoV-2 virus culture at an MOT of 0.1 for 48 hours. Cell viability was determined using the CellTiter-Glo® 2.0 Cell Viability Assay following the manufacturer's instructions. Briefly, Nluc substrate was diluted in cell lysis buffer and 100 μl was added to supernatant-pretreated, virally-infected Vero-E6-Luc2⁺ cells to assess cell viability. The resulting bioluminescence was measured on a microplate reader (Perkin Elmer, EnVision™ Multilabel Plate Reader).

Antiviral therapeutic effect of the ES effector cell with IFN-β. 24 hours after plating 1×10⁶ cells in 3 ml in complete RPMI media, ES effector cell with IFN-β were electrically stimulated for 1 hour with 30 V, 20 Hz and 2 msec pulse duration. Concomitantly, a monolayer of Vero-E6-Luc2+(20,000/well in a 96-well plate) were infected with SARS-CoV-2 virus culture at an MOT of 0.1 and incubated for 2 hours to allow virus attachment. After 2 hours, the virus inoculum was removed and 100 μl of serially diluted ES effector cell with IFN-β (2-fold) cells were immediately added to the wells in triplicate.

Recombinant human IFN-β₁a (1 μg/mL) and non-infected Vero-E6-Luc2⁺ cells were used as controls. After 48 hours of co-culture, Vero-E6-Luc2⁺ cell viability was determined by assessing Luc2 activity using the One-Glo® assay kit, following manufacturer's instructions. The Luc2 enzyme substrate was diluted in the cell lysis reagent and added to the Vero-E6-Luc2+ cells in 96-well plate to assess Luc2 enzyme activity associated with the viable cell population. Following a 10 minute incubation, bioluminescence was read on a microplate reader.

Table 1 below provides the different ES effector cells and SEQ ID NOs. for the plasmids.

TABLE 1

| | Plasmid A | | Plasmid B | |
|---|---|---|---|---|
| | Electrical- | | Electrical- | |
| | Sensor | SEQ ID | Sensor | |
| | (Ca⁺² | NOs. of | (Auxiliary | SEQ ID NOs of |
| ES-Cell | Channel) | plasmids | Subunits) | plasmids |
| ES effector | — | SEQ ID | — | — |
| cell→Nluc | | NO: 11 | +β₃ | SEQ ID NO: 15 |
| | | | +α₂δ₁ ₊ β₃ | SEQ ID NO: 16 |
| | Cav1.2 | SEQ ID | — | — |
| | | NO: 12 | +β₃ | SEQ ID NO: 15 |
| | | | +α₂δ₁ ₊ β₃ | SEQ ID NO: 16 |
| | | | +α₂δ₁ ₊ β₂ | SEQ ID NO: 17 |
| | Cav1.3 | SEQ ID | — | — |
| | | NO: 13 | +β₃ | SEQ ID NO: 15 |
| | | | +α₂δ₁ ₊ β₃ | SEQ ID NO: 16 |
| ES effector | Cav1.2 | SEQ ID | +α₂δ₁ ₊ β₃ | SEQ ID NO: 15 |
| cell→IFN-β | | NO: 14 | +α₂δ₁ ₊ β₂ | SEQ ID NO: 17 |

Table 2 below provides a summary of different genetic elements listed in SEQ ID NOs: 1-17.

TABLE 2

| Genetic element | SEQ ID NO |
|---|---|
| Cav 1.2 | SEQ ID NO: 1 |
| Cav 1.3 | SEQ ID NO: 2 |
| β₃ | SEQ ID NO: 3 |
| β₂ | SEQ ID NO: 4 |
| α₂δ₁ | SEQ ID NO: 5 |
| Nluc | SEQ ID NO: 6 |
| GFP | SEQ ID NO: 7 |
| Nluc 2a GFP | SEQ ID NO: 8 |
| P2A | SEQ ID NO: 9 |
| IFN-β | SEQ ID NO: 10 |
| Plasmid 1 (control) | SEQ ID NO: 11 |
| Plasmid 2 | SEQ ID NO: 12 |
| Plasmid 3 | SEQ ID NO: 13 |
| Plasmid 4 | SEQ ID NO: 14 |
| Plasmid 5 | SEQ ID NO: 15 |
| Plasmid 6 | SEQ ID NO: 16 |
| Plasmid 7 | SEQ ID NO: 17 |

Various embodiments are implemented in accordance with the underlying Provisional Application Ser. No. 63/142,311, entitled “Electrically Activatable Cell Biofactory,” filed Jan. 27, 2021, to which benefit is claimed and which is fully incorporated herein by reference for its general and specific teachings. For instance, embodiments herein and/or in the Provisional Application can be combined in varying degrees (including wholly). Reference can also be made to the experimental teachings and underlying references provided in the underlying Provisional Application. Embodiments discussed in the Provisional Application are not intended, in any way, to be limiting to the overall technical disclosure, or to any part of the claimed disclosure unless specifically noted.

Although specific examples have been illustrated and described herein, a variety of alternate and/or equivalent implementations can be substituted for the specific examples shown and described without departing from the scope of the present disclosure. This application is intended to cover any adaptations or variations of the specific examples discussed herein.

1. A genetically engineered electrically-stimulated (ES) effector cell comprising an exogenous polynucleotide sequence that includes, in operative association:
an electrical-sensor element that encodes a voltage-gated calcium ion channel (CaV), wherein the CaV is configured to transition from a closed state to an open state in response to electrical stimulation;
an actuator element that encodes a transcription factor binding site that upregulates synthesis of an effector protein in response to the transition of the CaV to the open state; and
an effector element that encodes the effector protein, wherein, in response to the transition of the CaV to the open state, the genetically engineered ES effector cell is configured to activate and, to synthesize and secrete the effector protein.

2. The cell of claim 1, wherein the genetically engineered ES effector cell is configured to activate by causing an influx of Ca⁺² in response to the electrical stimulation and the transition of the CaV to the open state, and the influx of Ca⁺² activates the transcription factor binding site and causes the upregulation of the synthesis of the effector protein.

3. The cell of claim 1, wherein the genetically engineered ES effector cell comprises a T-cell, a natural killer (NK) cell, a pluripotent stem cell, a multipotent stem cell, an epithelial cell, or a K562 cell.

4. The cell of claim 1, wherein the effector element further encodes a signal peptide upstream from the effector protein that is non-native to the effector protein.

5. The cell of claim 1, wherein the effector protein is selected from the group consisting of:
a therapeutic protein, a detectable reporter peptide, a downstream signaling protein, and a combination thereof.

6. The cell of claim 1, wherein the exogenous polynucleotide sequence further encodes a detectable reporter peptide.

7. The cell of claim 6, wherein the detectable reporter peptide is selected from the group consisting of:
luciferase or a bioluminescent variant thereof, Green Fluorescent Protein (GFP) or a fluorescent variant thereof, and lacZ or a colorimetric variant thereof.

8. The cell of claim 1, wherein the CaV includes a pore-forming subunit configured to form a channel in a cell surface of the genetically engineered ES effector cell and a set of auxiliary subunits configured to regulate the transition of a channel opening of the CaV and traffic the CaV to the cell surface.

9. The cell of claim 1, wherein the effector protein includes a therapeutic protein selected from the group consisting of:
a Type-I interferon (IFN), a Type-III IFN, and a combination thereof.

10. The cell of claim 1, wherein the CaV includes a pore-forming subunit selected from the group consisting of:
CaV1.2, CaV1.3, and variants thereof.

11. The cell of claim 1, wherein the transcription factor binding site is selected from the group consisting of:
a nuclear factor of activated T-cell (NFAT) response element, a serum response element (SRE), and a cyclic AMP response element (CRE).

12. The cell of claim 1, wherein the effector protein includes a therapeutic protein configured to act on a Severe Acute Respiratory Syndrome Coronavirus 2 (SARS-CoV-2)-infected cell.

13. A population of genetically engineered electrically-stimulated (ES) effector cells, each of the genetically engineered ES effector cells of the population comprising an exogenous polynucleotide sequence that includes an actuator element, an effector element, and an electrical-sensor element, wherein:
the electrical-sensor element encodes a voltage-gated calcium ion channel (CaV) including a pore forming sub-unit and a set of auxiliary subunits, wherein the CaV is configured to transition from a closed state to an open state in response to electrical stimulation and the set of auxiliary subunits are configured to regulate the of the CaV channel and trafficking of the CaV to the surface of the genetically engineered ES effector cell;
the actuator element encodes a transcription factor binding site that upregulates synthesis of an effector protein in response to the transition of the CaV to the open state; and
the effector element encodes the effector protein operably, wherein, in response to the transition of the CaV to the open state, the genetically engineered ES effector cell is configured to activate and, to synthesize and secrete the effector protein.

14. The population of genetically engineered ES effector cells of claim 13, wherein the set of auxiliary subunits includes a α₂δ subunit and a β subunit.

15. The population of genetically engineered ES effector cells of claim 13, wherein the set of auxiliary subunits includes:
a α₂δ₁ subunit and a β₃ subunit.

16. The population of genetically engineered ES effector cells of claim 13, wherein the population of genetically engineered ES effector cells are configured to provide a calibrated amount of the effector protein as a function of the electrical stimulation applied and a duration of the electrical stimulation applied.

17. A method comprising:
contacting a plurality of cells with a volume of a genetically engineered electrically-stimulated (ES) effector cell, wherein the genetically engineered ES effector cell comprises a polynucleotide sequence that includes:
an electrical-sensor element that encodes a voltage-gated calcium ion channel (CaV) including a pore-forming subunit and a set of auxiliary subunits;
an actuator element that encodes a transcription factor binding site; and
an effector element that encodes an effector protein;
after contacting the plurality of cells with the volume of the genetically engineered ES effector cell, applying an electric field to the volume of the genetically engineered ES effector cell to electrically stimulate the CaV;
in response to the electrical stimulation,
causing the CaV to transition from a closed state to an open state;
initiating expression of the effector protein by the actuator element; and
secreting the effector protein by a signal peptide.

18. The method of claim 17, further including locating electrical circuitry including two electrodes coupled to a power supply proximal to the volume of the genetically engineered effector ES cell and applying the electric field by applying a voltage between the two electrodes.

19. The method of claim 18, wherein the amount of secreted effector protein is provided as a function of the electric field applied to the genetically engineered ES effector cell by the electrical stimulation and a duration of the electrical stimulation applied.

20. The method of claim 18, further including activating the genetically modified ES effector cell as a function of at least one of:
a voltage, a total duration, a pulse duration, and a frequency of the electrical stimulation.
